(19) United States
(12) Patent Application Publication
(10) Pub. No.: US 2026/0317337 A1
(43) Pub. Date: Oct. 15, 2026

(54) HETEROCYCLIC COMPOUNDS FOR IMAGING AGGREGATED ALPHA SYNUCLEIN IN THE SYNUCLEINOPATHIES

(71) Applicants: Mark SCHNEIDER, Philadelphia, PA (US); Chia-Ju HSIEH, Philadelphia, PA (US); Dinahlee Saturnino GUARINO, Philadelphia, PA (US); THE TRUSTEES OF THE UNIVERSITY OF PENNSYLVANIA, Philadelphia, PA (US); UNIVERSITY OF PITTSBURGH - OF THE COMMONWEALTH SYSTEM OF HIGHER EDUCATION, Pittsburgh, PA (US); WASHINGTON UNIVERSITY, St. Louis, MO (US)

(72) Inventors: Robert H. MACH, Wallingford, PA (US); James E. PETERSSON, Wynnewood, PA (US); Guilong TIAN, Philadelphia, PA (US); Ho Young KIM, Philadelphia, PA (US); Wai-Kit CHIA, Galloway, NJ (US); Thomas J. GRAHAM, Philadelphia, PA (US); Chester A. MATHIS, Pittsburgh, PA (US); Paul KOTZBAUER, Saint Louis, MO (US); Zhude TU, Saint Louis, MO (US); Mark SCHNEIDER, Philadelphia, PA (US); Chia-Ju HSIEH, Philadelphia, PA (US); Dinahlee Saturnino GUARINO, Philadelphia, PA (US)

(21) Appl. No.: 19/482,453

(22) PCT Filed: May 10, 2024
(86) PCT No.: PCT/US2024/028910

§ 371 Date: Nov. 7, 2025

Related U.S. Application Data
(60) Provisional application No. 63/501,345, filed on May 10, 2023.

(51) Int. Cl. C07D 487/08; A61K 39/395; A61K 51/04; A61P 25/16; A61P 25/28; C07B 59/00; C07D 205/04; C07D 205/12; C07D 213/74; C07D 213/75; C07D 239/48; C07D 401/12; C07D 403/12; C07D 413/12; C07D 417/12; C07D 471/10; C07D 487/04; C07D 487/10

(52) U.S. Cl. CPC C07D 487/08; A61K 39/395; A61K 51/0444; A61K 51/0455; A61K 51/0459; A61K 51/0468; A61P 25/16; A61P 25/28; C07B 59/002; C07D 205/04; C07D 205/12; C07D 213/74; C07D 213/75; C07D 239/48; C07D 401/12; C07D 403/12; C07D 413/12; C07D 417/12; C07D 471/10; C07D 487/04; C07D 487/10

(57) ABSTRACT

The present disclosure provides compounds of Formula (I) and (II), or pharmaceutically acceptable salts or isotopic variants thereof, wherein, R¹ to R⁷, X₁, X₂, R²⁰-R²⁵, R³¹-R³⁴, Z₁, Z₂, and a-e are defined herein. Also provided are methods of modulating alpha-synuclein in a patient in need thereof and methods of treating a neurodegenerative disorder in a patient in need thereof using one or more compounds described herein.

### CROSS-REFERENCE TO RELATED APPLICATIONS

This application claims the benefit of the priority of U.S. Provisional Patent Application No. 63/501,345, filed May 10, 2023, the disclosure of which is incorporated by reference herein.

### GOVERNMENT RIGHTS

This invention was made with government support under NS110456 awarded by the National Institutes of Health. The government has certain rights in the invention.

### TECHNICAL FIELD

The present disclosure provides heterocyclic compounds and uses thereof.

### BACKGROUND

Alpha-synuclein is a 140 amino acid protein whose function in cell biology is poorly understood. Post-translational modification and abnormal aggregation of this protein is an event in a family of neurodegenerative disorders referred to as the “synucleinopathies.” The most common synucleinopathy is Parkinson's disease (PD), which is characterized by the insoluble aggregates of alpha-synuclein (Asyn or αsyn) in the form of Lewy bodies (LBs) and Lewy neurites (LNs). Another synucleinopathy is multiple system atrophy (MSA), which is characterized by alpha-synuclein aggregates in the form of glial cell inclusions (GCIs) in the oligodendrocytes largely in the cerebellum, pons and medulla. Since the abnormal aggregation of alpha-synuclein is believed to play a causal role in PD and MSA, there has been a major effort to develop novel therapeutic strategies aimed at preventing or removing pathological forms of this protein from brain.

What is needed are alternate methods for monitoring and treatment neurodegenerative diseases.

### SUMMARY

In some aspects, the disclosure provides compounds of Formula I or a pharmaceutically acceptable salt or isotopic variant thereof, wherein R¹ to R⁷, X₁ and X₂ are defined herein.

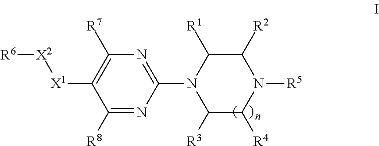

In other embodiments, the disclosure provides compounds of Formula II, or a pharmaceutically acceptable salt or isotopic variant thereof, wherein R²⁰-R²⁵, R³¹-R³⁴, Z₁, Z₂, and a-e are defined herein.

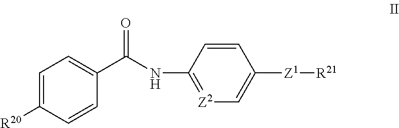

In further embodiments, the disclosure provides methods of modulating alpha-synuclein in a patient in need thereof, comprising administering an effective amount of a compound described herein to the patient. In certain aspects, the compound binds to alpha-synuclein.

In yet other embodiments, the disclosure provides methods of treating a neurodegenerative disorder in a patient in need thereof, comprising administering a pharmaceutical agent that clears aggregated alpha-synuclein from the brain of the patient; administering an effective amount of a compound described herein to the patient; and performing positron emission tomography imaging on the patient. In certain aspects, the compound has a low affinity for Ap.

### BRIEF DESCRIPTION OF THE DRAWINGS

FIGS. 1A and 1C are nuclear emulsion autoradiography images of a postmortem brain samples (A—not PD brain; C—MSA brain) after administration of ³H-Compound 103. FIGS. 1B and 1D are the same samples from FIGS. 1A and 1C after incubating sequentially with anti-phospho-α-syn (S129) antibody (p-syn/81A, BioLegend) and Alexa Fluor 488 goat anti-mouse secondary antibody to immunostain LBs and LNs. FIGS. 1E and 1G are nuclear emulsion autoradiography images of postmortem brain samples of (E—PD brain; G—not MSA brain) with ³H-Compound 43. FIGS. 1F and 1H are the same samples from FIGS. 1E and 1G after incubating sequentially with anti-phospho-α-syn (S129) antibody (p-syn/81A, BioLegend) and Alexa Fluor 488 goat anti-mouse secondary antibody to immunostain LBs and LNs.

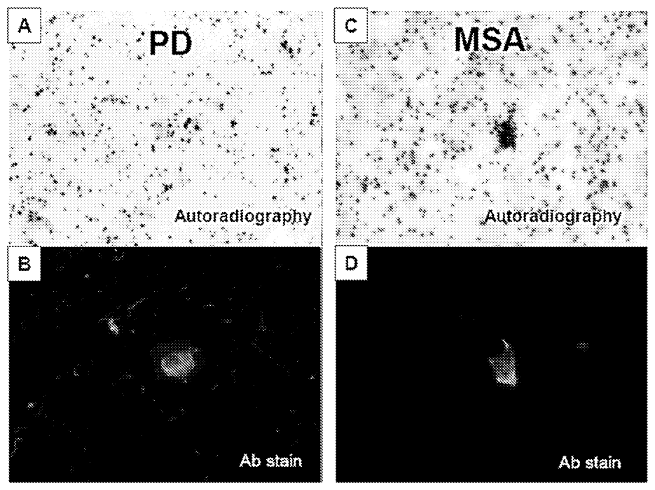

FIG. 2 are dynamic whole body PET images of ¹¹C-compound 103 in a non-human primate 1-90 minutes after administration ¹¹C-compound 103. This figure shows the high brain uptake and rapid washout of [¹¹C]-Compound 103.

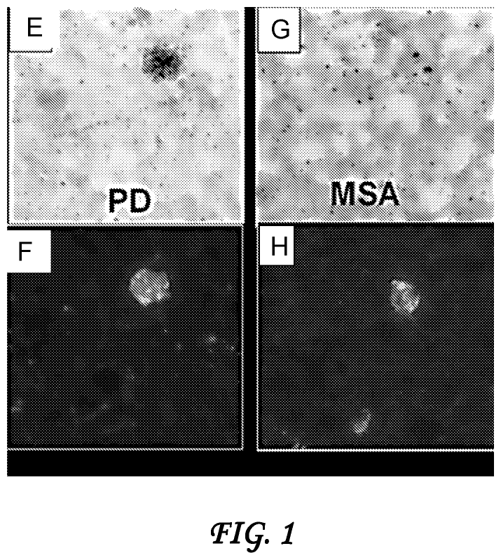

FIG. 3 are autoradiography images in tissue sections of ³H-Compound 103 and ³H-Compound 43 in PD, MSA and control brain. Neuropathological scores for Abeta, tau and Asyn are shown for each tissue section. FIG. 3 (top) shows the ability of [³H]-Compound 103 to image alpha synuclein in human postmortem samples of MSA and not PD and age-matched control. FIG. 3 (bottom) shows the ability of [³H]-Compound 43 to image alpha synuclein in human postmortem samples of PD and not MSA and age-matched control.

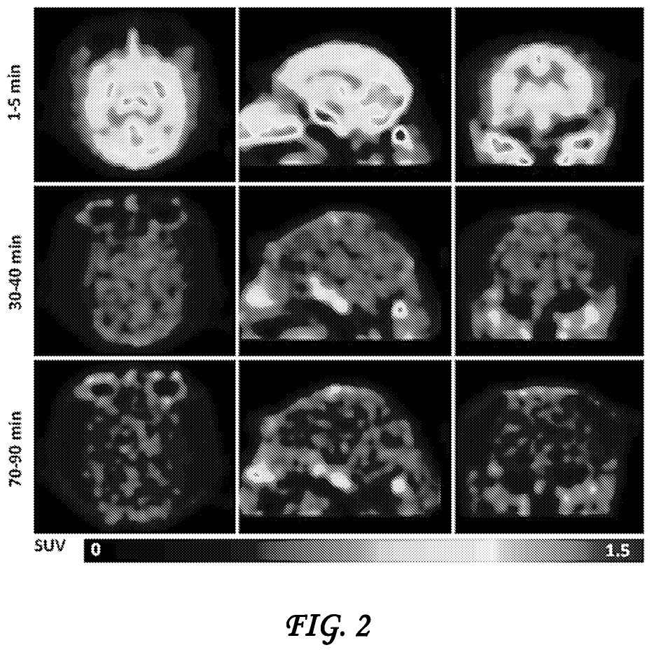

FIG. 4 is a dot plot showing the ratio of the specific binding signal for ³H-Compound 103 of pathology case (PD or MSA) vs. control for each radioligand. (PD: Control and MSA: Control). This figure shows the specificity of the radiotracers shown in FIG. 3.

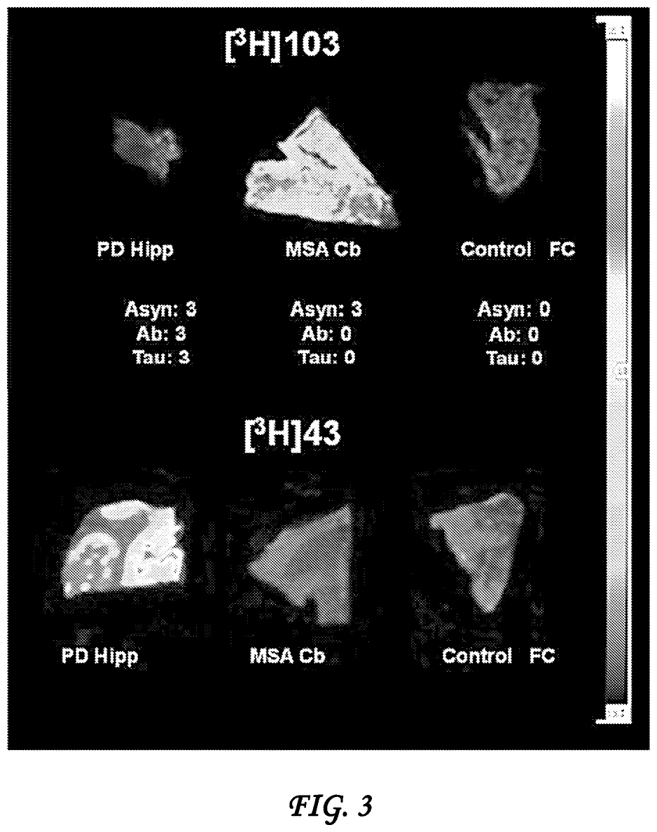

FIG. 5 is a dot plot showing the ratio of the specific binding signal for ³H-Compound 43 of pathology case (PD or MSA) vs. control for each radioligand. (PD: Control and MSA: Control). This figure shows the specificity of the radiotracers shown in FIG. 3.

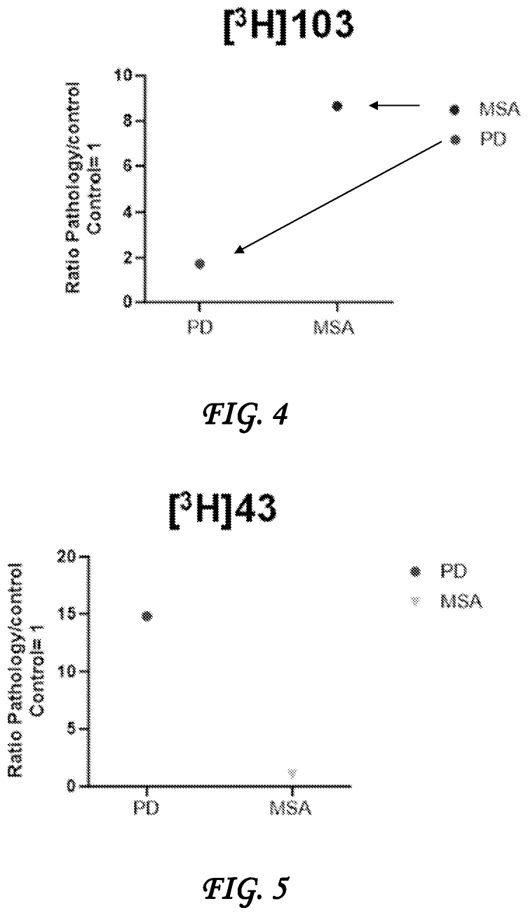

FIG. 6 are dynamic PET images (top) and time-activity curves for whole brain and different brain regions (bottom) of ¹¹C-compound 103 in a non-human primate and show the high uptake and rapid washout of [¹¹C]-compound 103.

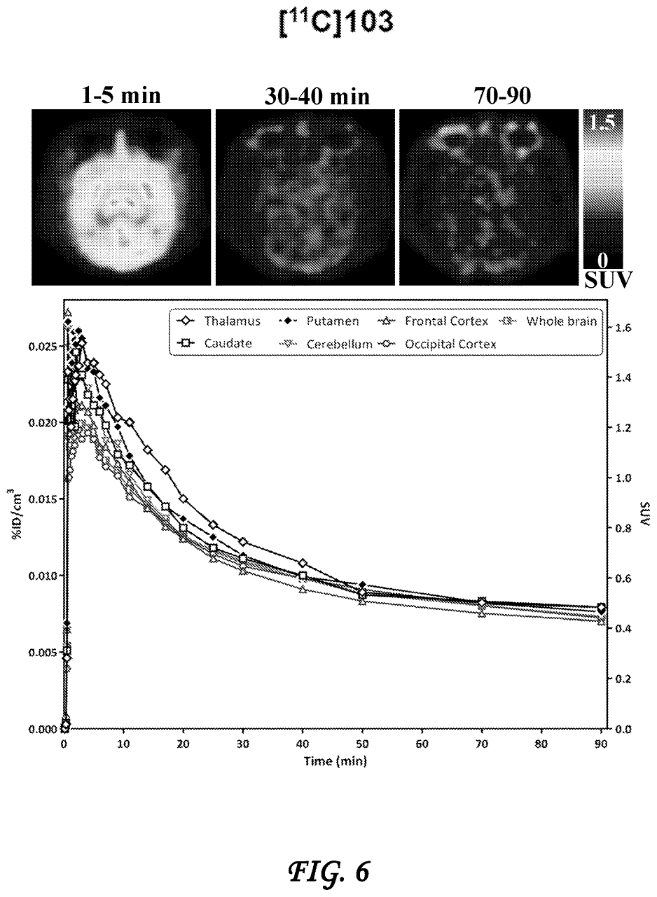

FIG. 7 are dynamic PET images (top) and time-activity curves (bottom) of ¹¹C-compound 43 in a non-human primate and show the high uptake and rapid washout of [¹¹C]-compound 43.

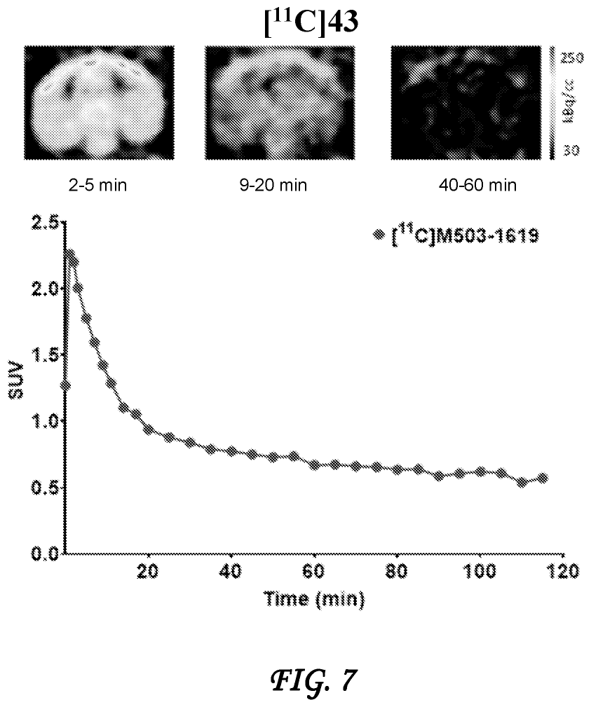

### DETAILED DESCRIPTION OF ILLUSTRATIVE EMBODIMENTS

Described herein is the radiosynthesis, in vitro binding, and in vivo evaluation of compounds that bind with high affinity to Asyn. Advantageously, the compounds disclosed herein have a low affinity for Abeta. Abeta is an off-target binding site that may interfere with imaging alpha synuclein aggregates in the synucleinopathies.

In the present disclosure the singular forms “a,” “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about.” Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

As used herein, the term “administering” means providing a pharmaceutical agent, i.e., a compound described herein, or composition, i.e., a pharmaceutical compositions described herein, to a subject, and includes, but is not limited to, administering by a medical professional and self-administering.

As used herein, the phrase “pharmaceutically acceptable” refers to compounds, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term:compound(s) of formula I” includes those compounds of “formula I,” as well as compounds of any of the formula I subgenera. The term “compound(s) of formula II” includes those compounds of “formula II,” as well as compounds of any of the formula I) subgenera.

As used herein, the term “subject” or “patient” means a human or non-human animal selected for treatment or therapy.

As used herein, the term “treating” a disease in a subject or “treating” a subject having or suspected of having a disease refers to subjecting the subject to a pharmaceutical treatment, e.g., the administration of an agent, such that at least one symptom of the disease is decreased or prevented from worsening.

As used herein, the term “isotopic variant” refers to a compound that contains unnatural proportions of isotopes at one or more of the atoms that constitute such compound. In some embodiments, an “isotopic variant” of a compound can be radiolabeled, that is, contain one or more non-radioactive isotopes and/or one radioactive isotope. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. In other embodiments, the compounds are radiolabeled with a non-radioactive isotope such as for example, deuterium (²H or D), carbon-13 (¹³C), nitrogen-15 (¹⁵N), or the like. In other embodiments, the compounds are, additionally or alternatively, labeled with one radioactive isotope. In some embodiments, the radioactive isotopes emit electrons, alpha (α), gamma (γ) or beta (β⁻ or β⁺) particles. Radioactive labels include, without limitation, tritium (³H), carbon-11 (¹¹C), carbon-14 (¹⁴C), fluorine-18 (¹⁸F), iodine-123 (¹²³I), iodine-124 (¹²⁴I), iodine-125 (¹²⁵I), iodine-131 (¹³¹I) bromine-76 (⁷⁶Br), bromine-77 (⁷⁷Br), oxygen-15 (¹⁵O), nitrogen-13 (¹³N), or astatine-211 (²¹¹At) or combinations thereof. Thus, radiolabeled compounds of the disclosure can be used in diagnostic methods such imaging. Imaging methods include, without limitation, single-photon emission computed tomography (SPECT) or Positron Emission Topography (PET) studies. In some embodiments, the radiolabel is ³H or ¹⁴C. In further embodiments, the compound is radiolabeled with ³H, ¹⁴C, and/or ¹²³I and may be used in SPECT. In other embodiments, the compound is radiolabeled with a β⁺ emitting isotopes, such as ¹¹C, ¹⁸F, ¹⁵O, ¹³N ¹²⁴I, or ⁷⁶Br and may be useful in PET. In further embodiments, the compound is radiolabeled with ¹⁸F or ¹¹C and may be useful in PET. In yet other embodiments, the compound is labeled with ¹²³I and may be useful in SPECT. In further embodiments, the compounds may be radiolabeled with electron emitting isotopes, such as ¹²³I, or ⁷⁷Br. In yet other embodiments, the compounds are radiolabeled with alpha particle emitting radiolabels such as ²¹¹At. In still further embodiments, the compounds are radiolabeled with β⁻ emitting isotopes such as ¹³¹I.

All isotopic variants of the compounds of the disclosure, radioactive or not, are intended to be encompassed within the scope of the disclosure.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. Within the present disclosure, any open valency appearing on a carbon, oxygen, or nitrogen atom in any structure described herein indicates the presence of a hydrogen atom. Where a chiral center exists in a structure, but no specific stereochemistry is shown for that center, both enantiomers, separately or as a mixture, are encompassed by that structure. The methods for the determination of stereochemistry and the separation of stereoisomers are well-known in the art.

The term “alkyl” as used herein, alone or in combination, refers to an aliphatic or cyclic hydrocarbon radical containing from 1 to about 20 carbon atoms, preferably from 2 to 6, more preferably from 2 to 4, carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 2-methylcyclopentyl, and 1-cyclohexylethyl. An alkyl described herein may be substituted as described herein.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. Examples of alkynyl radicals include, without limitation, ethynyl, propynyl, hydroxypropynyl, butyn-1-yl, butyn-2-yl, pentyn-1-yl, 3-methylbutyn-1-yl, hexyn-2-yl, and the like. An alkynyl described herein may be substituted as described herein.

The term “amino,” as used herein, alone or in combination, refers to —NRR,′ wherein R and R,′ are, independently, hydrogen, alkyl, acyl, heteroalkyl, aryl, cycloalkyl, heteroaryl, or heterocycloalkyl, any of which can themselves be optionally substituted.

The term “aryl,” as used herein, alone or in combination, means a carbocyclic aromatic system containing one, two or three rings wherein such rings can be attached together in a pendant manner or can be fused. The term “aryl” embraces aromatic radicals such as phenyl, naphthyl, anthracenyl, phenanthryl, indanyl, indenyl, annulenyl, azulenyl, tetrahydronaphthyl, and biphenyl. An aryl may be substituted as described herein.

The term “heteroaryl,” as used herein, alone or in combination, refers to 3- to 7-membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals.

Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like. A heteroaryl may be substituted as described herein.

The term “halogen,” “halo,” or “hal,” as used herein, alone or in combination, refers to fluorine, chlorine, bromine, or iodine.

The term “optionally substituted” means the anteceding group can be substituted or unsubstituted. When substituted, the substituents of an “optionally substituted” group can include, for example, one or more substituents independently selected from the following groups or a particular designated set of groups, alone or in combination: alkyl, alkenyl, alkynyl, cycloalkyl, aryl, alkoxy (such as methoxy), hydroxyalkyl, oxo, cyano, halogen, hydroxy, amino, nitro (NO₂), or thiol (SH). In some embodiments, the substituent is C₁₋₆alkyl. In other embodiments, the substituent is C₂₋₆alkenyl. In further embodiments, the substituent is C₂₋₆alkynyl. In yet other embodiments, the substituent is C₃₋₈cycloalkyl. In still further embodiments, the substituent is aryl. In other embodiments, the substituent is C₁₋₆alkoxy (such as methoxy). In further embodiments, the substituent is hydroxyalkyl, such as CH₂OH or CH₂CH₂OH. In yet other embodiments, the substituent is oxo. In still further embodiments, the substituent is cyano (CN). In other embodiments, the substituent is halo (F, or Cl, or Br). In further embodiments, the substituent is hydroxy (OH). In still other embodiments, the substituent is amino (NH₂). In yet further embodiments, the substituent is nitro (NO₂). In other embodiments, the substituent is thiol (SH).

### Compounds of Formula I

According to the disclosure, compounds of Formula I are described:

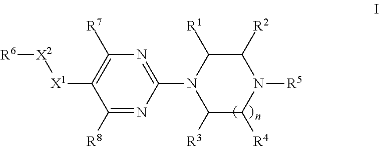

According to the disclosure, n is 1 or 2. In some embodiments, n is 1. In other embodiments, n is 2.

According to the disclosure, R¹ to R⁴ are H. In some embodiments, R¹ and R³ are H and R² and R⁴ are joined to form —C₁₋₆alk-. In other embodiments, R² and R⁴ are joined to form —CH₂—. In further embodiments, R² and R⁴ are joined to form —CH₂CH₂—. among others. In yet other embodiments, R² and R⁴ are H and R¹ and R³ are joined to form —C₁₋₆alk-. In still further embodiments, R¹ and R³ are joined to form —CH₂—. In other embodiments, R¹ and R³ are joined to form —CH₂CH₂—. In further embodiments, R¹ to R⁴ are H.

According to the disclosure, R⁵ is optionally substituted aryl or optionally substituted heteroaryl. In some embodiments, R⁵ is optionally substituted aryl. In further embodiments, R⁵ is optionally substituted phenyl. In further embodiments, R⁵ is optionally substituted heteroaryl. In yet other embodiments, R⁵ is optionally substituted N-containing heteroaryl. In still further embodiments, R⁵ is optionally substituted pyridyl, optionally substituted isoquinolinyl, optionally substituted benzoxazolyl, optionally substituted benzthiazolyl, or optionally substituted quinoxalinyl. In other embodiments, R⁵ is optionally substituted pyridyl. In further embodiments, R⁵ is optionally substituted isoquinolinyl. In yet other embodiments, R⁵ is optionally substituted benzoxazolyl. In still further embodiments, R⁵ is optionally substituted benzthiazolyl. In other embodiments, R⁵ is optionally substituted quinoxalinyl. In further embodiments, R⁵ is substituted with alkoxy such as methoxy.

According to the disclosure, X¹ is absent or —C(O)—. In some embodiments, X¹ is absent. In other embodiments, X¹ is —C(O)—.

According to the disclosure, X² absent, O, or NH. In some embodiments, X² is absent. In other embodiments, X² is O. In further embodiments, X² is NH.

According to the disclosure R⁶ is H, CN, C₁₋₆alkyl, C₁₋₆alk-OH, or optionally substituted phenyl. In some embodiments, R⁶ is H. In other embodiments, R⁶ is CN. In further embodiments, R⁶ is C₁₋₆alkyl. In yet other embodiments, R⁶ is methyl, ethyl, propyl, butyl, pentyl, or hexyl. In still further embodiments, R⁶ is methyl. In other embodiments, R⁶ is C₁₋₆alkOH. In further embodiments, R⁶ is CH₂OH, CH₂CH₂OH, propyl-OH, butyl-OH, pentyl-OH, or hexyl-OH. In yet other embodiments, R⁶ is optionally substituted phenyl. In further embodiments, R⁶ is

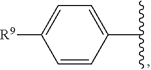

wherein R⁹ is optionally substituted C₁₋₆alkyl, optionally substituted C₁₋₆alkoxy, halo, optionally substituted C₁₋₆haloalkyl, or optionally substituted C₁₋₆haloalkoxy. In some aspects, R⁹ is optionally substituted C₁₋₆alkyl such as methyl, ethyl, propyl, butyl, pentyl, or hexyl, or such as CH₃. In other aspects, R⁹ is optionally substituted C₁₋₆alkoxy such as methoxy, ethoxy, propoxy, butoxy, pentoxy, or hexoxy, or such as methoxy. In further aspects, R⁹ is halo such as Br, Cl, or F, or such as F. In still further aspects, R⁹ is optionally substituted C₁₋₆haloalkyl such as CF₃. In yet other aspects, R⁹ is optionally substituted C₁₋₆haloalkoxy such as OCF₃, OCH₂CH₂F, or OCH₂CH(OH)CH₂F.

According to the disclosure, R⁷ and R⁸ are, independently, H, halo, or NH₂. In some embodiments, R⁷ is H. In other embodiments, R⁷ is halo such as Cl, F, or Br. In further embodiments, R⁷ is F. In further embodiments, R⁷ is NH₂. In yet other embodiments, R⁸ is H. In still further embodiments, R⁸ is halo such as Cl, F, or Br. In other embodiments, R⁸ is F. In further embodiments, R⁸ is NH₂.

In some aspects, the compound is of Formula I-A, or a pharmaceutically acceptable salt or isotopic variant thereof, wherein R¹-R⁸, X², and n are defined herein:

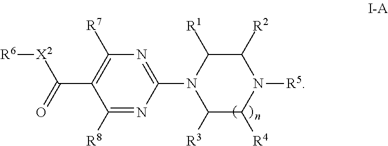

In other aspects, the compound is of Formula I-B, or a pharmaceutically acceptable salt or isotopic variant thereof, wherein R¹-R⁵, R⁷, R⁸, and n are defined herein:

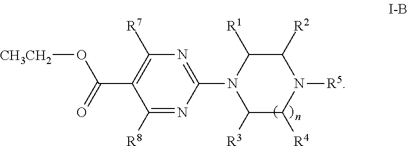

In further aspects, the compound is of Formula I-C, or a pharmaceutically acceptable salt or isotopic variant thereof, wherein R¹-R⁸ and n are defined herein:

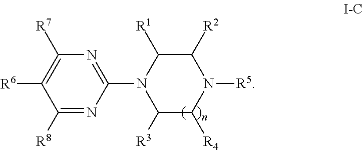

In yet other aspects, the compound is of Formula I-D, or a pharmaceutically acceptable salt or isotopic variant thereof, wherein R¹-R⁸ and n are defined herein:

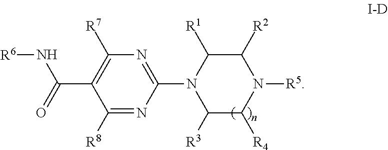

In still other aspects, the compound is of Formula I-E, or a pharmaceutically acceptable salt or isotopic variant thereof, wherein R¹-R⁵, R⁷, R⁸, and n are defined herein:

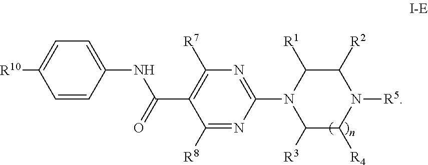

In this structure, R¹⁰ is C₁₋₆alkoxy such as methoxy, ethoxy, propoxy, butoxy, pentoxy, or hexoxy, or such as methoxy. In further aspects, R¹⁰ is halo such as Br, Cl, or F, or such as F. In still further aspects, R¹⁰ is C₁₋₆haloalkyl such as CF₃. In yet other aspects, R¹⁰ is C₁₋₆haloalkoxy such as OCF₃, OCH₂CH₂F, or OCH₂CH(OH)CH₂F.

According to the disclosure, the compound is:
ethyl 4-amino-2-(3-(pyridin-2-yl)-3,8-diazabicyclo[3.2.1]octan-8-yl)pyrimidine-5-carboxylate (1);
ethyl 4-amino-2-(3-(isoquinolin-3-yl)-3,8-diazabicyclo[3.2.1]octan-8-yl)pyrimidine-5-carboxylate (2);
ethyl 4-amino-2-(3-(quinolin-3-yl)-3,8-diazabicyclo[3.2.1]octan-8-yl)pyrimidine-5-carboxylate (3);
ethyl 4-amino-2-(8-(pyridin-2-yl)-3,8-diazabicyclo[3.2.1]octan-3-yl)pyrimidine-5-carboxylate (4);
ethyl 4-amino-2-(8-(isoquinolin-3-yl)-3,8-diazabicyclo[3.2.1]octan-3-yl)pyrimidine-5-carboxylate (5);
ethyl 4-amino-2-(8-(quinolin-3-yl)-3,8-diazabicyclo[3.2.1]octan-3-yl)pyrimidine-5-carboxylate (6);
ethyl 4-amino-2-(4-(pyridin-2-yl)-1,4-diazepan-1-yl)pyrimidine-5-carboxylate (7);
ethyl 4-amino-2-(4-(quinolin-3-yl)-1,4-diazepan-1-yl)pyrimidine-5-carboxylate (8);
ethyl 4-amino-2-(4-(pyridin-2-yl)piperazin-1-yl)pyrimidine-5-carboxylate (9);
ethyl 4-amino-2-(4-(benzo[d]oxazol-2-yl)piperazin-1-yl)pyrimidine-5-carboxylate (10);
ethyl 4-amino-2-(4-(benzo[d]thiazol-2-yl)piperazin-1-yl)pyrimidine-5-carboxylate (11);
ethyl 4-amino-2-(3-(benzo[d]thiazol-2-yl)-3,8-diazabicyclo[3.2.1]octan-8-yl)pyrimidine-5-carboxylate (12);
ethyl 4-amino-2-(4-(isoquinolin-3-yl)piperazin-1-yl)pyrimidine-5-carboxylate (13);
ethyl 4-amino-2-(4-(quinolin-3-yl)piperazin-1-yl)pyrimidine-5-carboxylate (14);
ethyl 4-amino-2-(4-(quinolin-2-yl)piperazin-1-yl)pyrimidine-5-carboxylate (15);
ethyl 4-amino-2-(4-(quinoxalin-2-yl)piperazin-1-yl)pyrimidine-5-carboxylate (16);
ethyl 4-amino-2-(4-(4-methoxyphenyl)piperazin-1-yl)pyrimidine-5-carboxylate (17);
4-amino-N-(4-methoxyphenyl)-2-(3-(pyridin-2-yl)-3,8-diazabicyclo[3.2.1]octan-8-yl)pyrimidine-5-carboxamide (18);
4-amino-2-(3-(isoquinolin-3-yl)-3,8-diazabicyclo[3.2.1]octan-8-yl)-N-(4-methoxyphenyl)pyrimidine-5-carboxamide (19);
4-amino-N-(4-methoxyphenyl)-2-(3-(quinolin-3-yl)-3,8-diazabicyclo[3.2.1]octan-8-yl)pyrimidine-5-carboxamide (20);
4-amino-2-(8-(isoquinolin-3-yl)-3,8-diazabicyclo[3.2.1]octan-3-yl)-N-(4-methoxyphenyl)pyrimidine-5-carboxamide (21);
4-amino-N-(4-methoxyphenyl)-2-(8-(quinolin-3-yl)-3,8-diazabicyclo[3.2.1]octan-3-yl)pyrimidine-5-carboxamide (22);
4-amino-N-(4-methoxyphenyl)-2-(4-(pyridin-2-yl)-1,4-diazepan-1-yl)pyrimidine-5-carboxamide (23);
4-amino-2-(4-(isoquinolin-3-yl)-1,4-diazepan-1-yl)-N-(4-methoxyphenyl)pyrimidine-5-carboxamide (24);
4-amino-N-(4-methoxyphenyl)-2-(4-(quinolin-3-yl)-1,4-diazepan-1-yl)pyrimidine-5-carboxamide (25);
4-amino-2-(4-(benzo[d]oxazol-2-yl)piperazin-1-yl)-N-(4-methoxyphenyl)pyrimidine-5-carboxamide (26);
4-amino-2-(4-(benzo[d]thiazol-2-yl)piperazin-1-yl)-N-(4-methoxyphenyl)pyrimidine-5-carboxamide (27);
4-amino-N-(4-methoxyphenyl)-2-(4-(pyridin-2-yl)piperazin-1-yl)pyrimidine-5-carboxamide (28);
4-amino-2-(4-(isoquinolin-3-yl)piperazin-1-yl)-N-(4-methoxyphenyl)pyrimidine-5-carboxamide (29);
4-amino-N-(4-methoxyphenyl)-2-(4-(quinolin-3-yl)piperazin-1-yl)pyrimidine-5-carboxamide (30);
4-amino-N-(4-methoxyphenyl)-2-(4-(quinolin-2-yl)piperazin-1-yl)pyrimidine-5-carboxamide (31);
4-amino-N-(4-methoxyphenyl)-2-(4-(quinoxalin-2-yl)piperazin-1-yl)pyrimidine-5-carboxamide (32);
4-amino-N-(4-methoxyphenyl)-2-(4-(4-methoxyphenyl)piperazin-1-yl)pyrimidine-5-carboxamide (33);
4-amino-N-(4-fluorophenyl)-2-(4-(pyridin-2-yl)piperazin-1-yl)pyrimidine-5-carboxamide (34);
4-amino-2-(4-(pyridin-2-yl)piperazin-1-yl)-N-(4-(trifluoromethyl)phenyl)pyrimidine-5-carboxamide (35);
4-amino-2-(4-(pyridin-2-yl)piperazin-1-yl)-N-(4-(trifluoromethoxy)phenyl)pyrimidine-5-carboxamide (36);
2-hydroxyethyl 4-amino-2-(4-(pyridin-2-yl)piperazin-1-yl)pyrimidine-5-carboxylate (37);
(4-amino-2-(4-(pyridin-2-yl)piperazin-1-yl)pyrimidin-5-yl)methanol (38);
5-methyl-2-(4-(pyridin-2-yl)piperazin-1-yl)pyrimidin-4-amine (39);
2-(4-(pyridin-2-yl)piperazin-1-yl)pyrimidin-4-amine (40);
4-amino-2-(4-(pyridin-2-yl)piperazin-1-yl)pyrimidine-5-carbonitrile (41);
N-(4-methoxyphenyl)-2-(4-(pyridin-2-yl)piperazin-1-yl)pyrimidine-5-carboxamide (42);
ethyl 4-amino-2-(4-(pyridin-4-yl)piperazin-1-yl)pyrimidine-5-carboxylate (117);
4-amino-N-(4-methoxyphenyl)-2-(4-(pyridin-3-yl)piperazin-1-yl)pyrimidine-5-carboxamide (118);
ethyl 4-amino-2-(4-(pyrimidin-2-yl)piperazin-1-yl)pyrimidine-5-carboxylate (119);
4-amino-N-(4-methoxyphenyl)-2-(4-(pyrimidin-2-yl)piperazin-1-yl)pyrimidine-5-carboxamide (120);
ethyl 4-amino-2-(4-(pyrazin-2-yl)piperazin-1-yl)pyrimidine-5-carboxylate (121);
4-amino-N-(4-methoxyphenyl)-2-(4-(pyrazin-2-yl)piperazin-1-yl)pyrimidine-5-carboxamide (122);
ethyl 4-amino-2-(4-(thiazol-2-yl)piperazin-1-yl)pyrimidine-5-carboxylate (123);
4-amino-N-(4-methoxyphenyl)-2-(4-(thiazol-2-yl)piperazin-1-yl)pyrimidine-5-carboxamide (124);
ethyl 4-amino-2-(3-(benzo[d]thiazol-2-yl)-3,8-diazabicyclo[3.2.1]octan-8-yl)pyrimidine-5-carboxylate (125);
4-amino-N-(4-methoxyphenyl)-2-(8-(quinolin-3-yl)-3,8-diazabicyclo[3.2.1]octan-3-yl)pyrimidine-5-carboxamide (126);
4-amino-N-(2-fluoro-4-methoxyphenyl)-2-(4-(pyridin-2-yl)piperazin-1-yl)pyrimidine-5-carboxamide (127);
4-amino-N-(3-fluoro-4-methoxyphenyl)-2-(4-(pyridin-2-yl)piperazin-1-yl)pyrimidine-5-carboxamide (128);
2-hydroxyethyl 4-amino-2-(4-(pyridin-2-yl)piperazin-1-yl)pyrimidine-5-carboxylate (129);
N-(4-methoxyphenyl)-2-(4-(pyridin-2-yl)piperazin-1-yl)pyrimidine-5-carboxamide (130);
4-amino-N-(4-hydroxyphenyl)-2-(4-(pyridin-2-yl)piperazin-1-yl)pyrimidine-5-carboxamide (131);
4-amino-N-(4-(2-fluoroethoxy)phenyl)-2-(4-(pyridin-2-yl)piperazin-1-yl)pyrimidine-5-carboxamide (132); or
4-amino-2-(4-(benzo[d]oxazol-2-yl)piperazin-1-yl)-N-(4-(2-fluoroethoxy)phenyl)pyrimidine-5-carboxamide (133);
   or a pharmaceutically acceptable salt or isotopic variant thereof.

### Compounds of Formula H

According to the disclosure, compounds of Formula II, or pharmaceutically acceptable salts or isotopic variants thereof are provided:

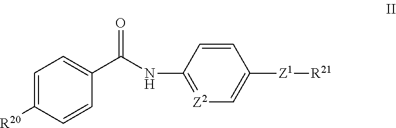

According to the disclosure, R²⁰ is optionally substituted C₁₋₆alkoxy or optionally substituted C₁₋₆haloalkoxy. In some embodiments, R²⁰ is C₁₋₆alkoxy, such as methoxy, ethoxy, propoxy, butoxy, pentoxy or hexoxy. In other embodiments, R²⁰ is OCH₃. In further embodiments, R²⁰ is C₁₋₆haloalkoxy, such as C₁₋₆fluoroalkoxy. In yet other embodiments, R²⁰ is OCH₂F or OCH₂CH₂F.

According to the disclosure, R²¹ is H, optionally substituted C₁₋₆alkyl, optionally substituted C₁₋₆alkoxy, optionally substituted heteroaryl or optionally substituted aryl. In some embodiments, R²¹ is H. In other embodiments, R²¹ is C₁₋₆alkyl, such as methyl, ethyl, propyl, butyl, pentyl, or hexyl. In further embodiments, R²¹ is methyl. In yet other embodiments, R²¹ is C₁₋₆alkoxy, such as methoxy, ethoxy, propoxy, butoxy, pentoxy or hexoxy. In still further embodiments, R²¹ is methoxy. In other embodiments, R²¹ is optionally substituted aryl, such as aryl, naphthyl, anthracenyl, phenanthryl, indanyl, indenyl, annulenyl, azulenyl, tetrahydronaphthyl, and biphenyl. In further embodiments, R²¹ is phenyl. In yet other embodiments, R²¹ is optionally substituted heteroaryl. In still further embodiments, R²¹ is a N-containing heteroaryl. In other embodiments, R²¹ is optionally substituted pyridyl, optionally substituted quinolinyl, or optionally substituted quinoxalinyl.

According to the disclosure, Z¹ is

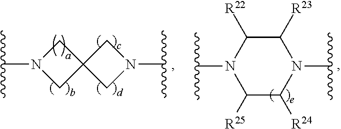

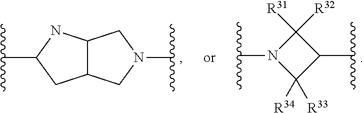

In other embodiments, Z¹ is

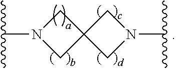

In other embodiments, Z¹ is

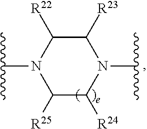

wherein e is 1 or 2. In some aspects, e is 1. In other aspects, e is 2. In further embodiments, Z¹ is

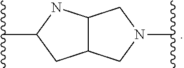 [SMILES: *C1CC2CN(*)CC2N1]

In still other embodiments, Z₁ is

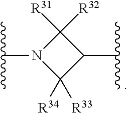

According to the disclosure, Z² is CH or N. In some embodiments, Z² is CH. In other embodiments, Z² is N.

According to the disclosure, a, b, c, d, and e are, independently, 1 or 2. In some embodiments, a is 1. In other embodiments a is 2. In further embodiments, b is 1. In yet other embodiments, b is 2. In still further embodiments, c is 1. In other embodiments, c is 2. In further embodiments, d is 1. In yet other embodiments, d is 2. In still further embodiments, a, b, c, and d are 1. In other embodiments, a and b are 1 and c and d are 2. In further embodiments, a and c are 1 and b and d are 2. In still other embodiments, a, b, and d are 2 and c is 1.

According to the disclosure, R²², R²³, R²⁴, and R²⁵ are H; or R²³ and R²⁴ are H and R²² and R²⁵ are joined to form —C₁₋₆alk-; or R²³ and R²⁵ are H and R²² and R²⁴ are joined to form —C₁₋₆alk-; or R²² and R²⁴ are H and R²³ and R²⁵ are joined to form —C₁₋₆alk-. In some embodiments, R²², R²³, R²⁴, and R²⁵ are H. In further embodiments, R²² and R²⁵ are H and R²³ and R²⁴ are joined to form —C₁₋₆alk-, such as —CH₂—, —CH₂CH₂—, or CH₂CH₂CH₂—. In other embodiments, R²³ and R²⁴ are H and R²² and R²⁵ are joined to form —C₁₋₆alk-, such as —CH₂—, —CH₂CH₂—, or CH₂CH₂CH₂—. In further embodiments, R²³ and R²⁵ are H and R²² and R²⁴ are joined to form —C₁₋₆alk-, such as —CH₂—, —CH₂CH₂—, or CH₂CH₂CH₂—. In yet other embodiments, R²² and R²⁴ are H and R²³ and R²⁵ are joined to form —C₁₋₆alk-, such as —CH₂—, —CH₂CH₂—, or CH₂CH₂CH₂—. In still further embodiments, R²² and R²⁵ are H and R²³ and R²⁴ are joined to form —C₁₋₆alk-, such as —CH₂—, —CH₂CH₂—, or CH₂CH₂CH₂—.

According to the disclosure, R³¹, R³², R³³, and R³⁴ are H; or R³³ and R³⁴ are H and R³¹ and R³² are joined to form a C₃₋₈cycloalkyl; or R³¹ and R³² are H and R³³ and R³⁴ are joined to form a C₃₋₈cycloalkyl. In some embodiments, R³¹, R³², R³³, and R³⁴ are H. In other embodiments, R³³ and R³⁴ are H and R³¹ and R³² are joined to form a C₃₋₈cycloalkyl, such as cyclopropyl, cyclobutyl, cyclopentyl, or cyclohexyl. In further embodiments, R³³ and R³⁴ are H and R³¹ and R³² are joined to form cyclopropyl. In yet other embodiments, R³³ and R³⁴ are H and R³¹ and R³² are joined to form a cyclobutyl. In still further embodiments, R³³ and R³⁴ are H and R³¹ and R³² are joined to form cyclopentyl. In other embodiments, R³³ and R³⁴ are H and R³¹ and R³² are joined to form cyclohexyl. In further embodiments, R³¹ and R³² are H and R³³ and R³⁴ are joined to form a C₃₋₈cycloalkyl, such as cyclopropyl, cyclobutyl, cyclopentyl, or cyclohexyl. In yet other embodiments, R³¹ and R³² are H and R³³ and R³⁴ are joined to form cyclopropyl. In still further embodiments, R³¹ and R³² are H and R³³ and R³⁴ are joined to form cyclobutyl. In other embodiments, R³¹ and R³² are H and R³³ and R³⁴ are joined to form cyclopentyl. In further embodiments, R³¹ and R³² are H and R³³ and R³⁴ are joined to form cyclohexyl.

In some embodiments, the compound is of formula II-A, wherein R²⁰, R²¹, and a-d are defined herein:

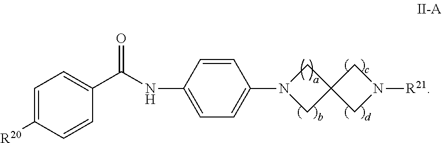

In other embodiments, the compound is of formula II-B, wherein R²⁰-R²⁵ and e are defined herein:

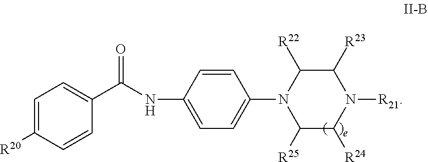

In further embodiments, the compound is of formula II-C:

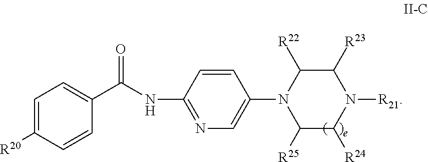

In yet other embodiments, the compound is:
4-methoxy-N-(4-(6-(pyridin-2-yl)-2,6-diazaspiro[3.3]heptan-2-yl)phenyl)benzamide (117);
4-methoxy-N-(4-(7-(pyridin-2-yl)-2,7-diazaspiro[3.5]nonan-2-yl)phenyl)benzamide (118);
4-methoxy-N-(4-(7-(3-nitrophenyl)-2,7-diazaspiro[3.5]nonan-2-yl)phenyl)benzamide (119);
4-methoxy-N-(4-(7-(pyridin-2-yl)-2,7-diazaspiro[4.4]nonan-2-yl)phenyl)benzamide (120);
4-methoxy-N-(4-(5-(pyridin-2-yl)hexahydropyrrolo[3,4-b]pyrrol-1(2H)-yl)phenyl)benzamide (100);
4-methoxy-N-(4-(2-(pyridin-2-yl)-2,8-diazaspiro[4.5]decan-8-yl)phenyl)benzamide (101);
4-methoxy-N-(4-(5-(pyridin-2-yl)-2,5-diazabicyclo[2.2.1]heptan-2-yl)phenyl)benzamide (102);
4-methoxy-N-(4-(3-(pyridin-2-yl)-3,8-diazabicyclo[3.2.1]octan-8-yl)phenyl)benzamide (103);
4-methoxy-N-(4-(3-(quinolin-3-yl)-3,8-diazabicyclo[3.2.1]octan-8-yl)phenyl)benzamide (104);
4-methoxy-N-(4-(3-(quinoxalin-2-yl)-3,8-diazabicyclo[3.2.1]octan-8-yl)phenyl)benzamide (105);
4-methoxy-N-(4-(3-(pyridin-2-yl)-3,9-diazabicyclo[3.3.1]nonan-9-yl)phenyl)benzamide (106);
4-methoxy-N-(4-(4-(pyridin-2-yl)-1,4-diazepan-1-yl)phenyl)benzamide (107);
N-(4-(4-(5-chloropyridin-2-yl)piperazin-1-yl)phenyl)-4-methoxybenzamide (108);
4-methoxy-N-(5-(4-(pyridin-2-yl)piperazin-1-yl)pyridin-2-yl)benzamide (109);
4-methoxy-N-(5-(3-(pyridin-2-yl)-3,8-diazabicyclo[3.2.1]octan-8-yl)pyridin-2-yl)benzamide (110);
4-methoxy-N-(5-(4-(pyridin-2-yl)-1,4-diazepan-1-yl)pyridin-2-yl)benzamide (111);
4-methoxy-N-(6-(3-(pyridin-2-yl)-3,8-diazabicyclo[3.2.1]octan-8-yl)pyridin-3-yl)benzamide (112);
4-methoxy-N-(4-(4-methylpiperazin-1-yl)phenyl)benzamide (113);
N-(4-(1-azaspiro[3.5]nonan-1-yl)phenyl)-4-methoxybenzamide (114);
4-methoxy-N-(4-(3-methoxyazetidin-1-yl)phenyl)benzamide (115); or
4-(2-fluoroethoxy)-N-(4-(3-(pyridin-2-yl)-3,8-diazabicyclo[3.2.1]octan-8-yl)phenyl)benzamide (116);
   or a pharmaceutically acceptable salt or isotopic variant thereof.

### Pharmaceutically Acceptable Salts of the Compounds

The compounds described herein can exist as salts, such as therapeutically acceptable salts. In some embodiments, the salt is an acid addition salts. Suitable salts include those formed with both organic and inorganic acids. Such acid addition salts will normally be pharmaceutically acceptable. However, salts of non-pharmaceutically acceptable salts can be of utility in the preparation and purification of the compound in question. For a more complete discussion of the preparation and selection of salts, refer to PharmaceuticalSalts: Properties, Selection, and Use (Stahl, P. Heinrich. Wiley-VCHA, Zurich, Switzerland, 2002).

As used herein, the phrase “salts” refers to the inorganic and organic salts of compounds. Some salts of the disclosure may be pharmaceutically acceptable salts, which are generally non-toxic inorganic and organic salts of compounds. Acid addition salts include, but are not limited to hydrochloride, hydrobromide, hydroiodide, nitrate, sulfate, bisulfate, phosphate, acid phosphate, isonicotinate, carbonate, bicarbonate, acetate, lactate, salicylate, citrate, tartrate, propionate, butyrate, pyruvate, oxalate, malonate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, trifluoromethanesulfonate, and pamoate (i.e., 1,1′-methylene-bis-(2-hydroxy-3-naphthoate)) salts.

The term “therapeutically acceptable salt,” as used herein, represents salts or zwitterionic forms of the compounds of the present invention which are water or oil-soluble or dispersible and therapeutically acceptable. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

### Pharmaceutical Compositions

While it is possible for compounds described herein to be administered as the raw chemical, it is also possible to include them in pharmaceutical compositions. Accordingly, pharmaceutical compositions are provided and comprise a compound or a pharmaceutically acceptable salt or isotopic variant thereof, together with one or more pharmaceutically acceptable excipient thereof. The excipient must be “acceptable,” i.e., compatible with other components of the pharmaceutical composition and not deleterious to the recipient thereof. Any of the well-known techniques, excipients, and excipients can be used as the excipient and as understood in the art. See, e.g., Remington's Pharmaceutical Sciences. The pharmaceutical compositions can be manufactured in a manner that is itself known, e.g., by means of conventional mixing, dissolving, granulating, dragee-making, levigating, emulsifying, encapsulating, entrapping or compression processes, for example.

In some embodiments, “pharmaceutically acceptable excipient” is a pharmaceutically-acceptable material, composition or vehicle involved in carrying or transporting an agent from one organ, or portion of the body, to another organ, or portion of the body. Each excipient must be “acceptable,” i.e., compatible with other components of the composition. Some examples of excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The pharmaceutical compositions include those suitable for oral, parenteral (including subcutaneous, intradermal, intramuscular, intravenous, intraarticular, and intramedullary), intraperitoneal, transmucosal, transdermal, rectal or topical (including dermal, buccal, sublingual and intraocular) administration. The pharmaceutical compositions can be presented in unit dosage form and can be prepared by any of the methods well known in the art.

Pharmaceutical compositions suitable for oral administration can be presented as discrete units such as capsules (e.g., push-fit, soft, sealed), sachets, tablets, powders, granules, solutions, suspensions, emulsions, bolus, or pastes.

The pharmaceutical compositions can be formulated for parenteral administration by injection, e.g., by bolus injection or continuous infusion. The pharmaceutical compositions can take such forms as suspensions, solutions or emulsions in oily or aqueous vehicles, and can contain formulatory agents such as suspending, stabilizing and/or dispersing agents. The formulations can be presented in unit-dose or multi-dose containers, e.g., sealed ampoules and vials, and can be stored in powder form or in a freeze-dried (lyophilized).

Pharmaceutical compositions for parenteral administration include aqueous and non-aqueous (oily) sterile injection solutions of the compounds described herein which can contain antioxidants, buffers, bacteriostats and solutes which render the formulation isotonic with the blood of the intended recipient; and aqueous and non-aqueous sterile suspensions which can include suspending agents and thickening agents. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

The pharmaceutical compositions can also be formulated for rectal administration, e.g., suppositories or retention enema.

The pharmaceutical compositions can also be formulated in vaginal compositions as gels, suppositories, or as dendrimers conjugates.

The pharmaceutical compositions can be administered topically. Pharmaceutical compositions suitable for topical administration include liquid or semi-liquid preparations suitable for penetration through the skin such as gels, liniments, lotions, creams, ointments or pastes.

### Methods of Treatment

The disclosure also provides methods of modulating alpha-synuclein in a patient in need thereof. The inventors found that the compounds described herein bind to alpha-synuclein. In some embodiments, the compounds bind with high affinity to Asyn. The compounds disclosed herein also have a low affinity for Abeta.

The methods include administering an effective amount of a compound described herein to the patient. In some embodiments, the compound is of formula I. In other embodiments, the compound is of formula II.

An “effective amount” means an amount or dose sufficient to generally bring about the desired therapeutic benefit in patients in need of such treatment for the designated disease, disorder, or condition. Effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

These effective amounts may be provided on regular schedule, i.e., daily, weekly, monthly, or yearly basis or on an irregular schedule with varying administration days, weeks, months, etc. Alternatively, the effective amount to be administered may vary. In some embodiments, the effective amount for the first dose is higher than the effective amount for one or more of the subsequent doses. In other embodiments, the effective amount for the first dose is lower than the effective amount for one or more of the subsequent doses.

The disclosure also provides noninvasive imaging techniques for measuring alpha-synuclein aggregates. Thus, these techniques are valuable tools for evaluating the efficacy of antibodies in preventing the formation of lewy bodies (LB), lewy neurites (LN), glial cytoplasmic inclusions (GCI), or combinations thereof in Parkinson's disease and multiple system atrophy (MSA).

The methods may also include performing imaging, such as positron emission tomography, on the patient. In some embodiments, imaging measures the amount of aggregated alpha-synuclein. In other embodiments, binding of compounds of the disclosure may be monitored and/or measured using imaging.

The compounds described herein also are useful in treating neurodegenerative disorders in a patient in need thereof. The methods include administering a pharmaceutical agent that clears aggregated alpha-synuclein from the brain of the patient; administering an effective amount of a compound described here to the patient; and performing positron emission tomography imaging on the patient. Examples of pharmaceutical agents that clear aggregated alpha-synuclein include monoclonal antibodies, such as humanized antibodies. For Example, the pharmaceutical agent is a humanized monoclonal antibody raised towards alpha synuclein and UCB0599, a small molecule inhibitor of alpha synuclein misfolding. See, e.g., Prasad, Current Therapies in Clinical Trials of Parkinson's Disease: A 2021 Update. 2021. 14(8); and Smit, Phase 1/lb Studies of UCB0599, an Oral Inhibitor of a-Synuclein Misfolding, Including a Randomized Study in Parkinson's Disease. Movement Disorders, 2022. 37(10): p. 2045-2056, the disclosures of which are incorporated by reference herein.

In some embodiments, the neurodegenerative disorder is a synucleinopathy. The term “synucleinopathy” as used herein refers to neurodegenerative disorders that share a common pathologic lesion composed of aggregates of insoluble a-synuclein protein in selectively vulnerable populations of neurons and glia. In certain aspects, the neurodegenerative disorder is Parkinson's disease, multiple system atrophy, dementia with lewy bodies, Shy-Drager syndrome, striatonigral degeneration, olivopontocerebellar atrophy, or neurodegeneration with brain iron accumulation, type I (adult neuroaxonal dystrophy or Hallervorden-Spatz syndrome). In other aspects, the neurodegenerative disorder is Parkinson's disease. In further aspects, the neurodegenerative disorder is multiple system atrophy.

The compounds may also be used in detecting a neurodegenerative disorder. Thus, these methods include administering an effective amount of the compound to a subject and detecting the neurodegenerative disorder. The neurodegenerative disorder may be detected by performing imaging such as positron emission tomography. In some embodiments, the detection methods described herein may be performed using an isotopically-labeled compound described herein. In some embodiments, the detection methods are performed using a compound labeled with radioactive isotope. In other embodiments, the detection methods are performed using a compound labeled with ¹⁸F. In further embodiments, the detection methods are performed using a compound labeled with ¹¹C. In yet other embodiments, the detection methods are performed using a compound labeled with¹²³I.

Also provided herein are methods of monitoring treatment of neurodegenerative disorders in a subject by administering an antibody to the subject, administering an effective amount of a compound described herein to said subject, and performing positron emission tomography on the subject. In some embodiments, the monitoring methods described herein may be performed using an isotopically-labeled compound described herein.

The methods described herein may be performed by administering a compound described herein via a combination therapy in prior to, concurrently with, or subsequent to another medication such as an antibody. As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab)2, a Fd fragment, a Fv fragments, and dAb fragments) as well as complete antibodies. In some embodiments, the antibody is a monoclonal antibody. In other embodiments, the antibody targets Aβ aggregates. In further embodiments, the antibody is AN1792, gantenerumab, aducanumab, or bapineuzumab.

Such combination treatment may occur by administering compositions containing multiple active ingredients, as described above. In some embodiments, the compound and antibody are administered to the patient by one or more selected routes of administration sequentially. In other embodiments, an antibody is administered before treatment with a compound described herein. In further embodiments, an antibody is administered after treatment with a compound described herein. In still other embodiments, an antibody is administered during treatment with a compound described herein.

Aside from the antibody, the compounds described herein are optionally administered in methods with one or more other therapeutic ingredients. In some embodiments, the other therapeutic ingredient is known to treat a neurodegenerative disorder or reduces neuroinflammation. In other embodiments, the other therapeutic ingredient is amantadine, apomorphine, baclofen, carbidopa, carbidopa/levodopa, dantrolene, donepezil, entacapone, galantamine, levodopa, memantine, pramipexole, rasagiline, riluzole, rivastigmine, ropinirole, selegiline, tacrine, tetrabenazine, tizanidine, or tolcapone, or combinations thereof.

Desirably, the compounds disclosed herein have a low affinity for Aβ. The term “low affinity” as used herein refers to the binding affinity between the compounds of the disclosure and Aβ. In some embodiments, the compounds of the disclosure have less than an about 50% binding affinity to Aβ. In other embodiments, the compounds of the disclosure have less than an about 40, about 30, about 20, about 10, about 5, or about 1% binding affinity to Aβ.

### Kits

Also provided herein are kits or packages containing a compound or composition described herein. The kits may be organized to indicate a single formulation or combination of formulations to be taken at each desired time. The composition may also be sub-divided to contain appropriate quantities of the compound. For example, the unit dosage can be packaged compositions, e.g., packeted powders, vials, ampoules, prefilled syringes or sachets containing liquids.

Suitably, the kit contains packaging or a container with the compound formulated for the desired delivery route. Optionally, the kit contains instructions on dosing and an insert regarding the compound. Optionally, the kit may further contain instructions for monitoring circulating levels of product and materials for performing such assays including, e.g., reagents, well plates, containers, markers or labels, and the like. Such kits are readily packaged in a manner suitable for treatment of a desired indication. For example, the kit may also contain instructions for use of the delivery device. Other suitable components to include in such kits will be readily apparent to one of skill in the art, taking into consideration the desired indication and the delivery route. The doses are repeated daily, weekly, or monthly, for a predetermined length of time or as prescribed.

The compound or composition described herein can be a single dose or for continuous or periodic discontinuous administration. For continuous administration, a package or kit can include the compound in each dosage unit (e.g., solution, lotion, tablet, pill, or other unit described above or utilized in drug delivery). When the compound is to be delivered with periodic discontinuation, a package or kit can include placebos during periods when the compound is not delivered. When varying concentrations of a composition, of the components of the composition, or of relative ratios of the compound or other agents within a composition over time is desired, a package or kit may contain a sequence of dosage units, so varying.

A number of packages or kits are known in the art for the use in dispensing pharmaceutical agents for oral use. In one embodiment, the package has indicators for each period. In another embodiment, the package is a labeled blister package, dial dispenser package, or bottle.

The packaging means of a kit may itself be geared for administration, such as an inhalant, syringe, pipette, eye dropper, or other such like apparatus, from which the formulation may be applied to an infected area of the body, such as the lungs, injected into a subject, or even applied to and mixed with the other components of the kit.

The compound or composition of these kits also may be provided in dried or lyophilized forms. When reagents or components are provided as a dried form, reconstitution generally is by the addition of a suitable solvent. It is envisioned that the solvent also may be provided in another packaging means.

The kits may include a means for containing the vials in close confinement for commercial sale such as, e.g., injection or blow-molded plastic containers into which the desired vials are retained.

Irrespective of the number or type of packages, the kits also may include, or be packaged with a separate instrument for assisting with the injection/administration or placement of the ultimate complex composition within the body of an animal. Such an instrument may be an inhalant, syringe, pipette, forceps, measuring spoon, eye dropper or any such medically approved delivery means. Other instrumentation includes devices that permit the reading or monitoring of reactions in vitro.

In some embodiments, a pharmaceutical kit is provided and contains a compound as described herein. The compound may be in the presence or absence of one or more of the pharmaceutically acceptable excipients described above. The kit may optionally contain an antibody and/or instructions for administering the antibody and the compound to a subject having cancer.

In further embodiments, a pharmaceutical kit is provided and contains an antibody in a first dosage unit, one or more of a compound selected from those described herein in a second dosage unit, and one or more of the pharmaceutically acceptable excipients described above in a third dosage unit. The kit may optionally contain instructions for administering the antibody and/or compound to a subject having cancer.

### Aspects

Aspect 1. A compound of Formula I:

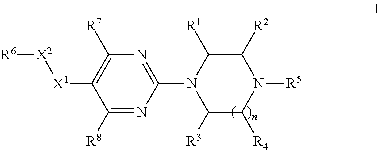

wherein:
n is 1 or 2;
R¹ to R⁴ are H; or R¹ and R³ are H and R² and R⁴ are joined to form —C₁₋₆alk-;
   or R² and R⁴ are H and R¹ and R³ are joined to form —C₁₋₆alk-;
R⁵ is optionally substituted aryl or optionally substituted heteroaryl;
X¹ is absent or —C(O)—;
X² absent, O, or NH;
R⁶ is H, CN, C₁₋₆alkyl, C₁₋₆alk-OH, or optionally substituted phenyl; and
R⁷ and R⁸ are, independently, H, halo, or NH₂;
   or a pharmaceutically acceptable salt or isotopic variant thereof.

Aspect 2. The compound of Aspect 1, wherein n is 1.

Aspect 3. The compound of Aspect 1, wherein n is 2.

Aspect 4. The compound of any one of the preceding Aspects, wherein R¹ to R⁴ are H.

Aspect 5. The compound of any one of Aspects 1-3, wherein R¹ and R³ are H and R² and R⁴ are joined to form —C₁₋₆alk-, such as —CH₂CH₂—.

Aspect 6. The compound of any one of Aspects 1-3, wherein R² and R⁴ are H and R¹ and R³ are joined to form —C₁₋₆alk-, such as —CH₂CH₂—.

Aspect 7. The compound of any one of the preceding Aspects, wherein R⁵ is optionally substituted aryl, such as phenyl.

Aspect 8. The compound of any one of the preceding Aspects, wherein R⁵ is optionally substituted heteroaryl such as a N-containing heteroaryl, or such as pyridyl, isoquinolinyl, benzoxazolyl, benzthiazolyl, or quinoxalinyl.

Aspect 9. The compound of Aspect 8, wherein R⁵ is optionally substituted pyridyl.

Aspect 10. The compound of Aspect 8, wherein R⁵ is optionally substituted isoquinolinyl.

Aspect 11. The compound of any one of the preceding Aspects, wherein X₁ is absent.

Aspect 12. The compound of any one of the preceding Aspects, wherein X₁ is —C(O)—.

Aspect 13. The compound of any one of the preceding Aspects, wherein X² is absent.

Aspect 14. The compound of any one of Aspects 1-12, wherein X² is O.

Aspect 15. The compound of any one of Aspects 1-12, wherein X² is NH.

Aspect 16. The compound of any one of the preceding Aspects, wherein R⁶ is H.

Aspect 17. The compound of any one of Aspects 1-15, wherein R⁶ is CN.

Aspect 18. The compound of any one of Aspects 1-15, wherein R⁶ is C₁₋₆alkyl, such as methyl, ethyl, propyl, butyl, pentyl, or hexyl, or such as methyl.

Aspect 19. The compound of any one of Aspects 1-15, wherein R⁶ is C₁₋₆alkOH, such as CH₂OH, CH₂CH₂OH, propyl-OH, butyl-OH, pentyl-OH, or hexyl-OH.

Aspect 20. The compound of any one of Aspects 1-15, wherein R⁶ is optionally substituted phenyl.

Aspect 21. The compound of any one of Aspects 1-15 or 20, wherein R⁶ is

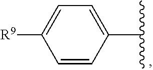

wherein R⁹ is optionally substituted C₁₋₆alkyl, optionally substituted C₁-6alkoxy, halo, optionally substituted C₁₋₆haloalkyl, or optionally substituted C₁₋₆haloalkoxy.

Aspect 22. The compound of any one of the preceding Aspects, wherein R⁷ is H.

Aspect 23. The compound of any one of Aspects 1-21, wherein R⁷ is halo such as Cl, F, or Br, or such as F.

Aspect 24. The compound of any one of the preceding Aspects, wherein R⁷ is H.

Aspect 25. The compound of any one of Aspects 1-21, wherein R⁷ is NH₂.

Aspect 26. The compound of any one of Aspects 1-21, wherein R⁸ is halo such as Cl, F, or Br, or such as F.

Aspect 27. The compound of any one of Aspects 1-21, wherein R⁸ is NH₂.

Aspect 28. The compound of any one of the preceding Aspects, wherein the compound is of Formula I-A:

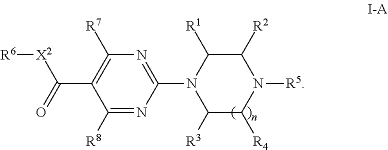

Aspect 29. The compound of any one of Aspects 1-21, wherein the compound is of Formula I-B:

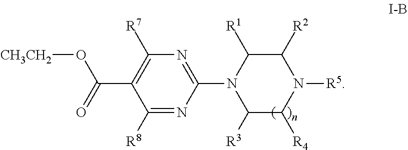

Aspect 30. The compound of any one of Aspects 1-21, wherein the compound is of Formula I-C:

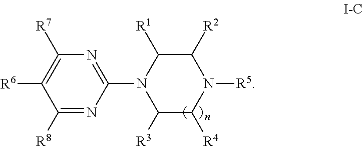

Aspect 31. The compound of any one Aspects 1-21, wherein the compound is of Formula I-D:

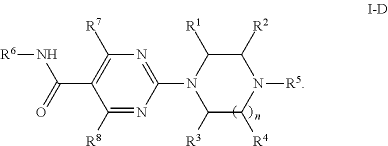

Aspect 32. The compound of any one of Aspects 1-21, wherein the compound is of Formula I-E:

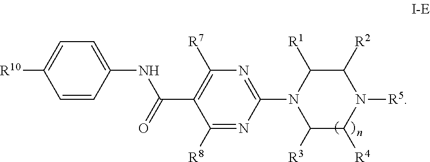

wherein, R¹⁰ is C₁₋₆alkoxy, halo, C₁₋₆trihaloalkyl, or C₁₋₆trihaloalkoxy.

Aspect 33. The compound of Aspect 1, that is:
4-amino-N-(4-fluorophenyl)-2-(4-(pyridin-2-yl)piperazin-1-yl)pyrimidine-5-carboxamide (34);
2-hydroxyethyl 4-amino-2-(4-(pyridin-2-yl)piperazin-1-yl)pyrimidine-5-carboxylate (37);
4-amino-2-(4-(pyridin-2-yl)piperazin-1-yl)-N-(4-(trifluoromethyl)phenyl)pyrimidine-5-carboxamide (35);
4-amino-2-(4-(pyridin-2-yl)piperazin-1-yl)-N-(4-(trifluoromethoxy)phenyl)pyrimidine-5-carboxamide (36);
2-(4-(pyridin-2-yl)piperazin-1-yl)pyrimidin-4-amine (40);
4-amino-2-(4-(pyridin-2-yl)piperazin-1-yl)pyrimidine-5-carbonitrile (41);
(4-amino-2-(4-(pyridin-2-yl)piperazin-1-yl)pyrimidin-5-yl)methanol (38);
5-methyl-2-(4-(pyridin-2-yl)piperazin-1-yl)pyrimidin-4-amine (39);
ethyl 4-amino-2-(3-(benzo[d]thiazol-2-yl)-3,8-diazabicyclo[3.2.1]octan-8-yl)pyrimidine-5-carboxylate (12);
ethyl 4-amino-2-(4-(benzo[d]oxazol-2-yl)piperazin-1-yl)pyrimidine-5-carboxylate (10);
ethyl 4-amino-2-(4-(benzo[d]thiazol-2-yl)piperazin-1-yl)pyrimidine-5-carboxylate (11);
4-amino-2-(4-(benzo[d]oxazol-2-yl)piperazin-1-yl)-N-(4-methoxyphenyl)pyrimidine-5-carboxamide (26);
4-amino-2-(4-(benzo[d]thiazol-2-yl)piperazin-1-yl)-N-(4-methoxyphenyl)pyrimidine-5-carboxamide (27);
ethyl 4-amino-2-(4-(4-methoxyphenyl)piperazin-1-yl)pyrimidine-5-carboxylate (17);
ethyl 4-amino-2-(4-(quinoxalin-2-yl)piperazin-1-yl)pyrimidine-5-carboxylate (16);
4-amino-N-(4-methoxyphenyl)-2-(4-(4-methoxyphenyl)piperazin-1-yl)pyrimidine-5-carboxamide (33);
4-amino-N-(4-methoxyphenyl)-2-(4-(quinoxalin-2-yl)piperazin-1-yl)pyrimidine-5-carboxamide (32);
N-(4-methoxyphenyl)-2-(4-(pyridin-2-yl)piperazin-1-yl)pyrimidine-5-carboxamide (42);
ethyl 4-amino-2-(4-(pyridin-2-yl)-1,4-diazepan-1-yl)pyrimidine-5-carboxylate (7);
4-amino-N-(4-methoxyphenyl)-2-(4-(pyridin-2-yl)-1,4-diazepan-1-yl)pyrimidine-5-carboxamide (23);
ethyl 4-amino-2-(4-(pyridin-2-yl)piperazin-1-yl)pyrimidine-5-carboxylate (9);
ethyl 4-amino-2-(8-(pyridin-2-yl)-3,8-diazabicyclo[3.2.1]octan-3-yl)pyrimidine-5-carboxylate (4);
ethyl 4-amino-2-(8-(isoquinolin-3-yl)-3,8-diazabicyclo[3.2.1]octan-3-yl)pyrimidine-5-carboxylate (5);
ethyl 4-amino-2-(8-(quinolin-3-yl)-3,8-diazabicyclo[3.2.1]octan-3-yl)pyrimidine-5-carboxylate (6);
4-amino-N-(4-methoxyphenyl)-2-(4-(pyridin-2-yl)piperazin-1-yl)pyrimidine-5-carboxamide (28);
ethyl 4-amino-2-(3-(pyridin-2-yl)-3,8-diazabicyclo[3.2.1]octan-8-yl)pyrimidine-5-carboxylate (1);
4-amino-2-(8-(isoquinolin-3-yl)-3,8-diazabicyclo[3.2.1]octan-3-yl)-N-(4-methoxyphenyl)pyrimidine-5-carboxamide (21);
4-amino-N-(4-methoxyphenyl)-2-(8-(quinolin-3-yl)-3,8-diazabicyclo[3.2.1]octan-3-yl)pyrimidine-5-carboxamide (22);
4-amino-N-(4-methoxyphenyl)-2-(3-(pyridin-2-yl)-3,8-diazabicyclo[3.2.1]octan-8-yl)pyrimidine-5-carboxamide (18);
ethyl 4-amino-2-(3-(isoquinolin-3-yl)-3,8-diazabicyclo[3.2.1]octan-8-yl)pyrimidine-5-carboxylate (2);
ethyl 4-amino-2-(3-(quinolin-3-yl)-3,8-diazabicyclo[3.2.1]octan-8-yl)pyrimidine-5-carboxylate (3);
ethyl 4-amino-2-(4-(quinolin-3-yl)-1,4-diazepan-1-yl)pyrimidine-5-carboxylate (8);
4-amino-2-(3-(isoquinolin-3-yl)-3,8-diazabicyclo[3.2.1]octan-8-yl)-N-(4-methoxyphenyl)pyrimidine-5-carboxamide (19);
4-amino-N-(4-methoxyphenyl)-2-(3-(quinolin-3-yl)-3,8-diazabicyclo[3.2.1]octan-8-yl)pyrimidine-5-carboxamide (20);
4-amino-N-(4-methoxyphenyl)-2-(4-(quinolin-3-yl)-1,4-diazepan-1-yl)pyrimidine-5-carboxamide (25);
4-amino-2-(4-(isoquinolin-3-yl)-1,4-diazepan-1-yl)-N-(4-methoxyphenyl)pyrimidine-5-carboxamide (24);
ethyl 4-amino-2-(4-(quinolin-3-yl)piperazin-1-yl)pyrimidine-5-carboxylate (14);
ethyl 4-amino-2-(4-(quinolin-2-yl)piperazin-1-yl)pyrimidine-5-carboxylate (15);
4-amino-N-(4-methoxyphenyl)-2-(4-(quinolin-3-yl)piperazin-1-yl)pyrimidine-5-carboxamide (30);
4-amino-N-(4-methoxyphenyl)-2-(4-(quinolin-2-yl)piperazin-1-yl)pyrimidine-5-carboxamide (31);
ethyl 4-amino-2-(4-(isoquinolin-3-yl)piperazin-1-yl)pyrimidine-5-carboxylate (13); or
4-amino-2-(4-(isoquinolin-3-yl)piperazin-1-yl)-N-(4-methoxyphenyl)pyrimidine-5-carboxamide (29);
   or a pharmaceutically acceptable salt or isotopic variant thereof.

Aspect 34. The compound of any one of the preceding Aspects, that is an isotopic variant.

Aspect 35. The compound of Aspect 34, that is labeled with one or more of a non-radioactive isotope such as ²H, ¹³C, or ¹⁵N, or such as ²H, or such as ¹³C, or such as ¹⁵N.

Aspect 36. The compound of Aspect 34, that is labeled with a radioactive isotope, such as ¹⁸F, ¹¹C, or 123I, or such as ¹⁸F, or such as ¹¹C, or such as ¹²³I.

Aspect 37. A pharmaceutical composition comprising one or more compounds of any one of Aspects 1-36 and a pharmaceutical excipient.

Aspect 38. A method of modulating alpha-synuclein in a patient in need thereof, comprising administering an effective amount of a compound of any one of Aspects 1-36 to the patient.

Aspect 39. The method of Aspect 38, wherein the compound binds to alpha-synuclein.

Aspect 40. The method of Aspect 38 or 39, further comprising performing positron emission tomography on the patient.

Aspect 41. A method of treating a neurodegenerative disorder in a patient in need thereof, comprising:
administering a pharmaceutical agent that clears aggregated alpha-synuclein from the brain of the patient;
administering an effective amount of a compound of any one of Aspects 1-36 to the patient; and
performing positron emission tomography imaging on the patient.

Aspect 42. The method of Aspect 41, wherein the neurodegenerative disorder is a synucleinopathy such as Parkinson's disease or multiple system atrophy.

v43. The method of Aspect 41 or 42, wherein the pharmaceutical agent is a monoclonal antibody.

Aspect 44. The method of any one of Aspects 41-43, wherein the imaging measures the amount of aggregated alpha-synuclein.

Aspect 45. The method of any one of Aspects 41-44, wherein the compound has a low affinity for Ap.

Aspect 46. A compound of Formula II:

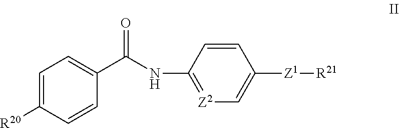

wherein:
R²⁰ is optionally substituted C₁₋₆alkoxy or optionally substituted C₁₋₆haloalkoxy;
R²¹ is H, optionally substituted C₁₋₆alkyl, optionally substituted C₁₋₆alkoxy, optionally substituted heteroaryl or optionally substituted aryl;

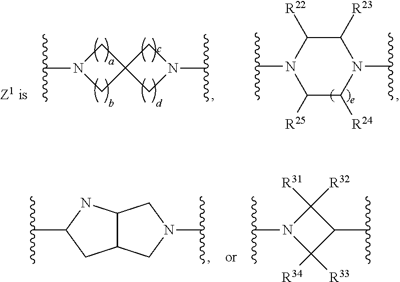

Z² is CH or N;
a, b, c, d, and e are, independently, 1 or 2;
R²², R²³, R²⁴, and R²⁵ are H; or R²³ and R²⁴ are H and R²² and R²⁵ are joined to form —C₁₋₆alk-; or R²³ and R²⁵ are H and R²² and R²⁴ are joined to form —C₁-6alk-; or R²² and R²⁴ are H and R²³ and R²⁵ are joined to form —C₁₋₆alk-; or R²² and R²⁵ are H and R²³ and R²⁴ are joined to form —C₁₋₆alk-;
R³¹, R³², R³³, and R³⁴ are H; or R³³ and R³⁴ are H and R³¹ and R³² are joined to form a C₃₋₈cycloalkyl; or R³¹ and R³² are H and R³³ and R³⁴ are joined to form a C₃₋₈cycloalkyl;
or a pharmaceutically acceptable salt isotopic variant thereof.

Aspect 47. The compound of Aspect 46, wherein R²⁰ is C₁₋₆alkoxy, such as OCH₃.

Aspect 48. The compound of Aspect 46, wherein R²⁰ is C₁₋₆haloalkoxy, such as C₁₋₆fluoroalkoxy, or such as OCH₂F or OCH₂CH₂F.

Aspect 49. The compound of any one of Aspects 46-48, wherein R²¹ is H.

Aspect 50. The compound of any one of Aspects 46-48, wherein R²¹ is C₁-6alkyl, such as methyl.

Aspect 51. The compound of any one of Aspects 46-48, wherein R²¹ is C₁-6alkoxy, such as methoxy.

Aspect 52. The compound of any one of Aspects 46-48, wherein R²¹ is optionally substituted aryl, such as phenyl.

Aspect 53. The compound of any one of Aspects 46-48, wherein R²¹ is optionally substituted heteroaryl, such as a N-containing heteroaryl, or such as pyridyl, quinolinyl, or quinoxalinyl.

Aspect 54. The compound of any one of Aspects 46-53, wherein Z¹ is

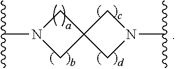

Aspect 55. The compound of Aspect 54, wherein a, b, c, and d are 1.

Aspect 56. The compound of Aspect 54, wherein a and b are 1 and c and d are 2.

Aspect 57. The compound of Aspect 54, wherein a and c are 1 and b and d are 2.

Aspect 58. The compound of Aspect 54, wherein a, b, and d are 2 and c is 1.

Aspect 59. The compound of any one of Aspects 46-58, wherein Z¹ is

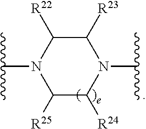

Aspect 60. The compound of Aspect 59, wherein e is 1.

Aspect 61. The compound of Aspect 59, wherein e is 2.

Aspect 62. The compound of Aspect 59, wherein R²², R²³, R²⁴, and R²⁵ are H.

Aspect 63. The compound of Aspect 59, wherein R²³ and R²⁴ are H and R²² and R²⁵ are joined to form —C₁₋₆alk-, such as —CH₂—, —CH₂CH₂—, or CH₂CH₂CH₂—.

Aspect 64. The compound of Aspect 59, wherein R²³ and R²⁵ are H and R²² and R²⁴ are joined to form —C₁₋₆alk-, such as —CH₂—, —CH₂CH₂—, or CH₂CH₂CH₂—.

Aspect 65. The compound of Aspect 59, wherein R²² and R²⁴ are H and R²³ and R²⁵ are joined to form —C₁₋₆alk-, such as —CH₂—, —CH₂CH₂—, or CH₂CH₂CH₂—.

Aspect 66. The compound of Aspect 59, wherein R²² and R²⁵ are H and R²³ and R²⁴ are joined to form —C₁₋₆alk-, such as —CH₂—, —CH₂CH₂—, or CH₂CH₂CH₂—.

Aspect 67. The compound of any one of Aspects 46-58, wherein Z¹ is

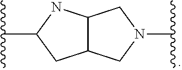 [SMILES: *C1CC2CN(*)CC2N1]

Aspect 68. The compound of any one of Aspects 46-58, wherein Z¹ is

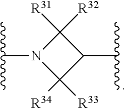

Aspect 69. The compound of Aspect 68, wherein R³¹, R³², R³³, and R³⁴ are H.

Aspect 70. The compound of Aspect 68, wherein R³³ and R³⁴ are H R³¹ and R³² are joined to form a C₃₋₈cycloalkyl, such as cyclopropyl, cyclobutyl, cyclopentyl, or cyclohexyl.

Aspect 71. The compound of Aspect 68, wherein R³¹ and R³² are H and R³³ and R³⁴ are joined to form a C₃₋₈cycloalkyl, such as cyclopropyl, cyclobutyl, cyclopentyl, or cyclohexyl.

Aspect 72. The compound of any one of Aspects 46-71, wherein Z² is CH.

Aspect 73. The compound of any one of Aspects 46-71, wherein Z² is N.

Aspect 74. The compound of Aspect 46, that is of formula II-A:

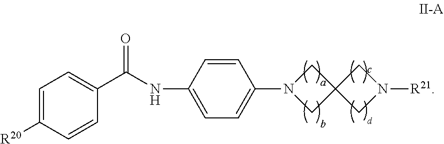

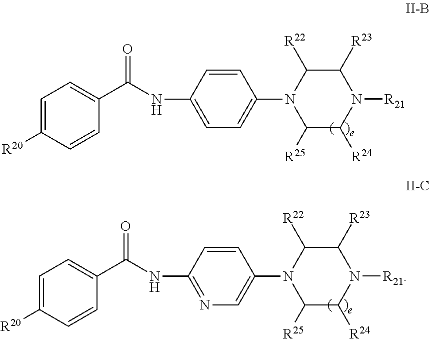

Aspect 76. The compound of Aspect 46, that is:
4-methoxy-N-(4-(6-(pyridin-2-yl)-2,6-diazaspiro[3.3]heptan-2-yl)phenyl)benzamide (117);
4-methoxy-N-(4-(7-(pyridin-2-yl)-2,7-diazaspiro[3.5]nonan-2-yl)phenyl)benzamide (118);
4-methoxy-N-(4-(7-(3-nitrophenyl)-2,7-diazaspiro[3.5]nonan-2-yl)phenyl)benzamide (119);
4-methoxy-N-(4-(7-(pyridin-2-yl)-2,7-diazaspiro[4.4]nonan-2-yl)phenyl)benzamide (120);
4-methoxy-N-(4-(5-(pyridin-2-yl)hexahydropyrrolo[3,4-b]pyrrol-1(2H)-yl)phenyl)benzamide (100);
4-methoxy-N-(4-(2-(pyridin-2-yl)-2,8-diazaspiro[4.5]decan-8-yl)phenyl)benzamide (101);
4-methoxy-N-(4-(5-(pyridin-2-yl)-2,5-diazabicyclo[2.2.1]heptan-2-yl)phenyl)benzamide (102);
4-methoxy-N-(4-(3-(pyridin-2-yl)-3,8-diazabicyclo[3.2.1]octan-8-yl)phenyl)benzamide (103);
4-methoxy-N-(4-(3-(quinolin-3-yl)-3,8-diazabicyclo[3.2.1]octan-8-yl)phenyl)benzamide (104);
4-methoxy-N-(4-(3-(quinoxalin-2-yl)-3,8-diazabicyclo[3.2.1]octan-8-yl)phenyl)benzamide (105);
4-methoxy-N-(4-(3-(pyridin-2-yl)-3,9-diazabicyclo[3.3.1]nonan-9-yl)phenyl)benzamide (106);
4-methoxy-N-(4-(4-(pyridin-2-yl)-1,4-diazepan-1-yl)phenyl)benzamide (107);
N-(4-(4-(5-chloropyridin-2-yl)piperazin-1-yl)phenyl)-4-methoxybenzamide (108);
4-methoxy-N-(5-(4-(pyridin-2-yl)piperazin-1-yl)pyridin-2-yl)benzamide (109);
4-methoxy-N-(5-(3-(pyridin-2-yl)-3,8-diazabicyclo[3.2.1]octan-8-yl)pyridin-2-yl)benzamide (110);
4-methoxy-N-(5-(4-(pyridin-2-yl)-1,4-diazepan-1-yl)pyridin-2-yl)benzamide (111);
4-methoxy-N-(6-(3-(pyridin-2-yl)-3,8-diazabicyclo[3.2.1]octan-8-yl)pyridin-3-yl)benzamide (112);
4-methoxy-N-(4-(4-methylpiperazin-1-yl)phenyl)benzamide (113);
N-(4-(1-azaspiro[3.5]nonan-1-yl)phenyl)-4-methoxybenzamide (114);
4-methoxy-N-(4-(3-methoxyazetidin-1-yl)phenyl)benzamide (115);
4-(2-fluoroethoxy)-N-(4-(3-(pyridin-2-yl)-3,8-diazabicyclo[3.2.1]octan-8-yl)phenyl)benzamide (116);
   or a pharmaceutically acceptable salt or isotopic variant thereof.

Aspect 77. The compound of any one of Aspects 46-76, that is an isotopic variant.

Aspect 78. The compound of Aspect 77, that is labeled with one or more of a non-radioactive isotope such as ²H, ¹³C, or ⁵N, or such as 2H, or such as ³C, or such as ¹⁵N.

Aspect 79. The compound of Aspect 77, that is labeled with a radioactive isotope, such as ¹⁸F, ¹¹C, or ¹²³I, or such as ¹⁸F, or such as ¹¹C, or such as ¹²³I.

Aspect 80. A pharmaceutical composition comprising one or more compounds of any one of Aspects 46-79 and a pharmaceutical excipient.

Aspect 81. A method of modulating alpha-synuclein in a patient in need thereof, comprising administering an effective amount of a compound of any one of Aspects 46-79 to the patient.

Aspect 82. The method of Aspect 81, wherein the compound binds to alpha-synuclein.

Aspect 83. The method of Aspect 81 or 82, further comprising performing positron emission tomography on the patient.

Aspect 84. A method of treating a neurodegenerative disorder in a patient in need thereof, comprising:
administering a pharmaceutical agent that clears aggregated alpha-synuclein from the brain of the patient;
administering an effective amount of a compound of any one of Aspects 46-79 to the patient; and
performing positron emission tomography imaging on the patient.

Aspect 85. The method of Aspect 84, wherein the neurodegenerative disorder is a synucleinopathy such as Parkinson's disease or multiple system atrophy.

Aspect 86. The method of Aspect 84 or 885, wherein the pharmaceutical agent is a monoclonal antibody.

Aspect 87. The method of any one of Aspects 84-86, wherein the imaging measures the amount of aggregated alpha-synuclein.

Aspect 88. The method of any one of Aspects 84-87, wherein the compound has a low affinity for Aβ.

This disclosure is further illustrated by the following examples, which should not be construed as limiting.

### EXAMPLES

### Example 1: Synthesis of Quinoline Related Compounds

1-(pyridin-2-yl)piperazine was purchased from Sigma-Aldrich. The amines for the synthesis of Compounds 10, 11, 15-17, 26, 27, and 31-33 were prepared as described in in Mach Org. Lett. 2016, 18, 5272-5275.

The general process for the synthesis of secondary 1-(pyridin-2-yl)piperazine analogs and related data.

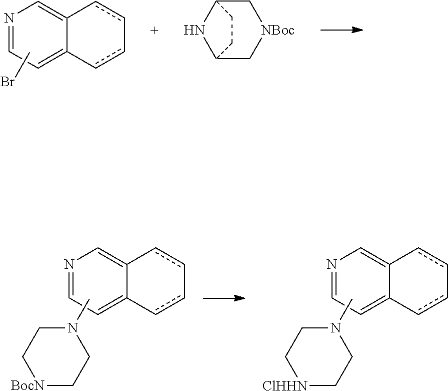

Step a: Amine (3.0 mmol, 1.0 equiv.), corresponding aryl bromide (3.0 mmol, 1.0 equiv.), Pd₂(dba)₃ (1.0 mol %), Ruphos (2.0 mol %) and ^{t}BuONa (4.0 equiv.) were mixed in a sealed vial and 1,4-dioxane (6 mL) was added. The mixture was kept stirring at 100° C. for 20 min. The mixture was diluted with DCM and filtered through a celite pad. The filtrate was condensed and the crude product was purified by flash column chromatography on silica gel, eluting with hexanes/ethyl acetate=4: 1 to 2: 1 to afford the desired product.

Step b: The Boc-protected amine was dissolved in DCM (10 mL). HCl in 1,4-dioxane (2 M, 4 mL) was added. The mixture was stirred at room temperature overnight. The solid was filtered out. The solid was collected and the crude products were used for the next step directly.

### Example 2: tert-Butyl 3-(pyridin-2-yl)-3,8-diazabicyclo[3.2.1]octane-8-carboxylate

This compound was prepared using the procedure of Example 1. ¹H NMR (400 MHz, CDCl₃) δ 8.17 (dd, J=5.0, 1.8 Hz, 1H), 7.48 (t, J=6.9 Hz, 1H), 6.63 (dd, J=6.9, 5.0 Hz, 1H), 6.57 (d, J=8.6 Hz, 1H), 4.36 (s, 2H), 3.96 (s, 1H), 3.82 (s, 1H), 3.09 (s, 2H), 1.95, 1.91 (m, 2H), 1.79-1.74 (m, 2H), 1.46 (s, 9H); ¹³C NMR (101 MHz, CDCl₃) δ 159.7, 153.7, 147.2, 138.0, 113.3, 107.0, 79.9, 53.6, 52.8, 50.6, 28.6, 27.8. LCMS: 290 [M+H]⁺. White solid, 676 mg, 78%.

### Example 3: tert-Butyl 3-(isoquinolin-3-yl)-3,8-diazabicyclo[3.2.1]octane-8-carboxylate

This compound was prepared using the procedure of Example 1. ¹H NMR (400 MHz, CDCl₃) δ 8.92 (s, 1H), 7.77 (d, J=8.2 Hz, 1H), 7.56 (d, J=8.3 Hz, 1H), 7.49 (ddd, J=8.3, 6.7, 1.3 Hz, 1H), 7.27-7.21 (m, 1H), 6.67 (s, 1H), 4.41 (s, 2H), 4.00 (d, J=39.5 Hz, 2H), 3.12 (s, 2H), 2.04-1.76 (m, 4H), 1.49 (s, 9H); ¹³C NMR (101 MHz, CDCl₃) δ 157.1, 153.8, 151.0, 138.9, 130.5, 127.7, 125.3, 123.6, 123.3, 98.4, 79.8, 53.8, 53.0, 51.3, 28.6. LCMS: 340 [M+H]+. White solid, 975 mg, 96%.

### Example 4: tert-Butyl 3-(quinolin-3-yl)-3,8-diazabicyclo[3.2.1]octane-8-carboxylate

This compound was prepared using the procedure of Example 1. 1H NMR (400 MHz, CDCl₃) δ 8.75 (d, J=2.9 Hz, 1H), 8.02-7.98 (m, 1H), 7.65 (dd, J=7.9, 1.7 Hz, 1H), 7.52-7.43 (m, 2H), 7.27 (d, J=3.0 Hz, 1H), 4.43 (s, 2H), 3.59-3.47 (m, 2H), 3.13 (s, 2H), 2.07-1.86 (m, 4H), 1.49 (s, 9H); ¹³C NMR (101 MHz, CDCl₃) δ 153.7, 144.7, 142.6, 142.2, 129.0, 128.6, 127.3, 126.5, 126.5, 116.0, 80.1, 28.6. LCMS: 340 [M+H]+. White solid, 975 mg, 96%.

### Example 5: tert-Butyl 8-(pyridin-2-yl)-3,8-diazabicyclo[3.2.1]octane-3-carboxylate

This compound was prepared using the procedure of Example 1. 1H NMR (400 MHz, CDCl₃) δ 8.18-8.11 (m, 1H), 7.48-7.39 (m, 1H), 6.62-6.51 (m, 2H), 4.49 (s, 2H), 3.80 (d, J=12.7 Hz, 1H), 3.65 (d, J=12.7 Hz, 1H), 3.21 (d, J=12.7 Hz, 1H), 3.11 (d, J=12.7 Hz, 1H), 2.00-1.91 (m, 2H), 1.82 (dd, J=16.4, 7.5 Hz, 2H), 1.43 (s, 9H); ¹³C NMR (101 MHz, CDCl₃) δ 157.2, 156.2, 148.6, 137.7, 113.2, 109.0, 79.7, 53.8, 53.4, 48.1, 46.8, 28.5, 27.1. LCMS: 290 [M+H]⁺. White solid, 736 mg, 86%.

### Example 6: tert-Butyl 8-(isoquinolin-3-yl)-3,8-diazabicyclo[3.2.1]octane-3-carboxylate

This compound was prepared using the procedure of Example 1. 1H NMR (400 MHz, CDCl₃) δ 8.94 (s, 1H), 7.77 (d, J=8.3 Hz, 1H), 7.57-7.46 (m, 2H), 7.27-7.22 (m, 1H), 6.75 (s, 1H), 4.65 (d, J=19.9 Hz, 2H), 3.86 (d, J=12.8 Hz, 1H), 3.72 (d, J=12.4 Hz, 1H), 3.30 (d, J=12.8 Hz, 1H), 3.21 (d, J=12.8 Hz, 1H), 2.07-2.02 (m, 2H), 1.88 (dd, J=16.6, 7.7 Hz, 2H), 1.45 (s, 9H); ¹³C NMR (101 MHz, CDCl₃) δ 156.2, 151.2, 139.1, 130.8, 127.8, 124.9, 123.4, 123.1, 100.9, 79.7, 54.3, 54.1, 47.9, 46.4, 28.4, 27.1, 27.0. LCMS: 340 [M+H]+. Light-yellow solid, 863 mg, 85%.

### Example 7: tert-Butyl 8-(quinolin-3-yl)-3,8-diazabicyclo[3.2.1]octane-3-carboxylate

This compound was prepared using the procedure of Example 1. 1H NMR (400 MHz, CDCl₃) δ 8.69 (d, J=3.0 Hz, 1H), 7.98-7.93 (m, 1H), 7.62-7.56 (m, 1H), 7.47-7.41 (m, 2H), 7.24 (d, J=2.7 Hz, 1H), 4.34 (d, J=15.7 Hz, 2H), 3.83 (d, J=12.9 Hz, 1H), 3.69 (d, J=12.9 Hz, 1H), 3.33 (d, J=12.9 Hz, 1H), 3.24 (d, J=12.9 Hz, 1H), 2.10-2.05 (m, 2H), 1.90 (dd, J=14.7, 7.3 Hz, 2H), 1.44 (s, 9H); ¹³C NMR (101 MHz, CDCl₃) δ 156.1, 143.6, 142.2, 140.3, 129.3, 129.1, 127.2, 126.1, 125.9, 116.4, 80.0, 55.0, 54.8, 47.2, 45.8, 28.5, 27.1, 26.9. LCMS: 340 [M+H]+. Light-yellow solid, 814 mg, 80%.

### Example 8: tert-Butyl 8-(benzo[d]thiazol-2-yl)-3,8-diazabicyclo[3.2.1]octane-3-carboxylate

This compound was prepared using the procedure of Example 1. 1H NMR (400 MHz, CDCl₃) δ 7.60 (dd, J=7.9, 1.2 Hz, 1H), 7.56 (d, J=8.1 Hz, 1H), 7.32-7.28 (m, 1H), 7.11-7.06 (m, 1H), 4.37 (s, 2H), 3.79 (s, 2H), 3.43 (s, 2H), 2.01-1.98 (m, 2H), 1.84-1.79 (m, 2H), 1.49 (s, 9H); ¹³C NMR (101 MHz, CDCl₃) δ 169.8, 153.5, 126.5, 126.3, 121.7, 120.8, 119.2, 111.4, 80.4, 53.8, 28.6, 27.5. LCMS: 346 [M+H]⁺. White solid, 640 mg, 62%.

### Example 9: tert-Butyl 4-(pyridin-2-yl)-1,4-diazepane-1-carboxylate

This compound was prepared using the procedure of Example 1. 1H NMR (400 MHz, CDCl₃) δ 8.08 (d, J=5.0 Hz, 1H), 7.37 (t, J=7.9 Hz, 1H), 6.50-6.42 (m, 2H), 3.72 (t, J=5.5 Hz, 2H), 3.65-3.48 (m, 4H), 3.28 (t, J=6.1 Hz, 1H), 3.18 (t, J=6.2 Hz, 1H), 1.94-1.88 (m, 2H), 1.36 (d, 9H). ¹³C NMR (101 MHz, CDCl₃) δ 157.33, 157.28, 155.3, 155.1, 148.2, 148.1, 137.4, 111.8, 105.63, 105.57, 79.4, 48.9, 48.5, 47.3, 47.1, 46.7, 46.2, 45.8, 28.4, 28.3, 25.3, 25.0. LCMS: 278 [M+H]+. Light-yellow oil, 673 mg, 81%.

### Example 10: tert-Butyl 4-(isoquinolin-3-yl)-1,4-diazepane-1-carboxylate

This compound was prepared using the procedure of Example 1. 1H NMR (400 MHz, CDCl₃) δ 8.87 (s, 1H), 7.72 (dd, J=8.2, 1.1 Hz, 1H), 7.51 (d, J=8.5 Hz, 1H), 7.44 (ddd, J=8.3, 6.6, 1.2 Hz, 1H), 7.16 (ddd, J=8.1, 6.6, 1.2 Hz, 1H), 6.60 (d, J=1.0 Hz, 1H), 3.88 (t, J=5.4 Hz, 2H), 3.81-3.72 (m, 2H), 3.66-3.57 (m, 2H), 3.34 (t, J=6.1 Hz, 1H), 3.23 (t, J=6.2 Hz, 1H), 2.09-2.00 (m, 2H), 1.39 (d, 9H); ¹³C NMR (101 MHz, CDCl₃) δ 155.5, 155.2, 154.4, 151.4, 151.3, 139.2, 130.3, 127.8, 127.8, 124.9, 122.7, 122.3, 96.6, 96.4, 79.5, 67.2, 49.6, 49.2, 47.7, 47.6, 47.0, 46.3, 46.0, 28.5, 28.4, 25.5, 24.9. LCMS: 328 [M+H]+. Light-yellow solid, 870 mg, 89%.

### Example 11: tert-Butyl 4-(quinolin-3-yl)-1,4-diazepane-1-carboxylate

This compound was prepared using the procedure of Example 1. 1H NMR (400 MHz, CDCl₃) δ 8.66 (d, J=3.0 Hz, 1H), 7.98-7.90 (m, 1H), 7.60 (q, J=4.0 Hz, 1H), 7.44-7.36 (m, 2H), 7.14 (s, 1H), 3.76-3.58 (m, 6H), 3.35 (t, J=6.0 Hz, 1H), 3.24 (t, J=6.2 Hz, 1H), 2.04 (q, J=6.3 Hz, 2H), 1.35 (d, 9H); ¹³C NMR (101 MHz, CDCl₃) δ 155.4, 155.0, 141.2, 141.0, 140.3, 140.1, 129.6, 128.8, 127.1, 126.0, 125.2, 112.2, 112.1, 79.9, 50.5, 50.3, 49.0, 48.2, 46.4, 46.3, 45.8, 28.5, 28.3, 25.5, 25.0. LCMS: 328 [M+H]+. Light-yellow solid, 900 mg, 92%.

### Example 12: tert-Butyl 4-(isoquinolin-3-yl)piperazine-1-carboxylate

This compound was prepared using the procedure of Example 1. 1H NMR (400 MHz, CDCl₃) δ 8.97 (s, 1H), 7.80 (dd, J=8.3, 1.1 Hz, 1H), 7.60 (dd, J=8.5, 1.1 Hz, 1H), 7.54 (t, J=6.8 Hz, 1H), 7.30 (t, J=6.8 Hz, 1H), 6.81 (s, 1H), 3.67-3.53 (m, 8H), 1.50 (s, 9H). ¹³C NMR (101 MHz, CDCl₃) δ 154.8, 150.6, 139.0, 1131.2, 131.0, 127.8, 125.4, 124.0, 123.6, 100.1, 80.0, 46.4, 43.5, 28.5. LCMS: 314 [M+H]+. Light-yellow solid, 836 mg, 89%.

### Example 13: tert-Butyl 4-(quinolin-3-yl)piperazine-1-carboxylate

This compound was prepared using the procedure of Example 1. 1H NMR (400 MHz, CDCl₃) δ 8.81-8.75 (m, 1H), 8.08-8.02 (m, 1H), 7.73-0.67 (m, 1H), 7.59-7.45 (m, 2H), 7.42-7.38 (m, 1H), 3.65 (t, J=5.0 Hz, 4H), 3.26 (s, 4H), 1.49 (s, 9H); ¹³C NMR (101 MHz, CDCl₃) δ 144.7, 128.8, 128.3, 126.7, 118.2, 80.2, 49.3, 43.4, 28.4. LCMS: 314 [M+H]+. Light-yellow solid, 808 mg, 86%.

### Example 14: General Process for Analogs of 4-Amino-N-(4-methoxyphenyl)-2-(4-(pyridin-2-yl)piperazin-1-yl)pyrimidine-5-carboxamide (28)

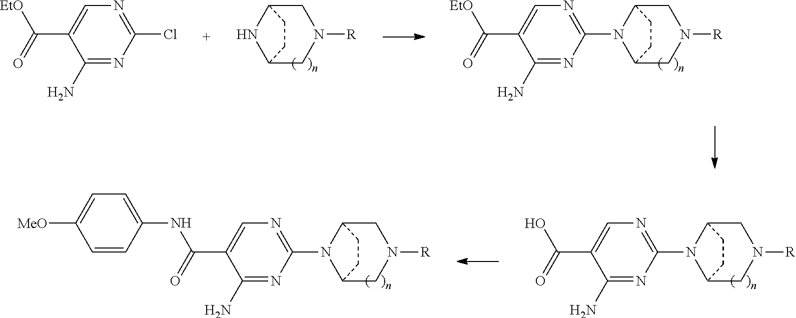

Step a: Ethyl 4-amino-2-chloropyrimidine-5-carboxylate (60 mg, 0.30 mmol) and the corresponding amine or the salt (0.45 mmol) was dissolved in 4 mL dry MeCN. Et₃N (91 mg, 0.90 mmol) was added to the solution. The mixture was stirred at room temperature overnight. Removed the solvent and the crude product was purified by flash column chromatography on silica gel, eluting with hexanes/ethyl acetate (with 5% 7N NH₃ in MeOH) (4:1 to 1:1) to afford the desired product as a solid.

Step b: The product (0.20 mmol) from the last step was dissolved in EtOH and H₂O (2 mL/2 mL). The mixture was heated to 60° C. for 4 h. After cooling to room temperature, EtOH was removed under reduced pressure. 1 N HCl was used to make the solution to pH=7. The precipitated solid was filtered out and dried on a high vacuum for 12 h. The crude product was used for the next step without further purification.

Step c: A solution of the crude product above, DIPEA (77 mg, 0.6 mmol) and HBTU (0.21 mmol) in 4 mL DMF was stirred for 15 min at room temperature. 4-methoxyaniline (26 mg, 0.21 mmol) in DMF (1 mL) was added to the solution. The mixture was stirred at room temperature overnight. The mixture was washed with 1 N Na₂CO₃ solution and extracted with DCM (3×15 mL) and dried over Na₂SO₄. Removed the solvent and the residue was purified by flash column chromatography on silica gel, eluting with 5% MeOH in DCM to afford the desired product as a solid.

### Example 15: Ethyl 4-amino-2-(3-(pyridin-2-yl)-3,8-diazabicyclo[3.2.1]octan-8-yl)pyrimidine-5-carboxylate (1)

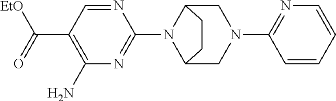 [SMILES: CCOC(=O)c1cnc(N2C3CCC2CN(c2ccccn2)C3)nc1N]

This compound was prepared using the procedure of Example 14 (R=pyridin-2-yl). 1H NMR (400 MHz, CDCl₃) δ 8.65 (s, 1H), 8.17 (d, J=4.7 Hz, 1H), 7.75 (brsm, 1H), 7.51-7.45 (m, 1H), 6.64-6.63 (m, 1H), 6.56 (d, J=8.6 Hz, 1H), 5.39 (brsm, 1H), 4.96 (s, 2H), 4.28 (q, J=7.1 Hz, 2H), 3.96 (d, J=11.8 Hz, 2H), 3.18 (d, J=12.0 Hz, 2H), 2.05-1.97 (m, 2H), 1.92-1.87 (m, 2H), 1.33 (t, J=7.1 Hz, 3H); ¹³C NMR (101 MHz, CDCl₃) δ 166.7, 163.6, 160.9, 159.8, 147.3, 138.0, 113.3, 106.9, 96.5, 60.4, 53.2, 50.5, 27.5, 14.5. LCMS: 355 [M+H]+; HRMS (ESI) calculated for C₁₈H₂₃N₆O₂+([M+H+]) 355.1877, found: 355.1876. White solid, 54 mg, 51%.

### Example 16: Ethyl 4-amino-2-(3-(isoquinolin-3-yl)-3,8-diazabicyclo[3.2.1]octan-8-yl)pyrimidine-5-carboxylate (2)

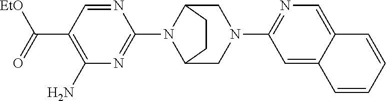 [SMILES: CCOC(=O)c1cnc(N2C3CCC2CN(c2cc4ccccc4cn2)C3)nc1N]

This compound was prepared using the procedure of Example 14 (R=isoquinolin-3-yl). 1H NMR (400 MHz, CDCl₃) δ 8.91 (s, 1H), 8.67 (s, 1H), 7.76 (d, J=8.1 Hz, 1H), 7.55 (d, J=8.3 Hz, 1H), 7.52-7.45 (m, 1H), 7.26-7.21 (m, 2H), 6.67 (s, 1H), 5.43 (brsm, 1H), 5.00 (s, 2H), 4.37-4.24 (m, 2H), 4.13-4.09 (m, 2H), 3.20 (dd, J=11.8, 2.2 Hz, 2H), 2.08-1.95 (m, 4H), 1.35 (t, J=7.1 Hz, 3H); ¹³C NMR (101 MHz, CDCl₃) δ 166.4, 163.3, 157.1, 151.0, 149.2, 138.8, 130.5, 127.7, 125.3, 123.7, 123.4, 98.5, 96.6, 60.7, 53.9, 51.4, 27.6, 14.5. LCMS: 405 [M+H]⁺; HRMS (ESI) calculated for C₂₂H₂₅N₆O₂+([M+H+]) 405.2034, found: 405.2049. White solid, 61 mg, 50%.

### Example 17: Ethyl 4-amino-2-(3-(quinolin-3-yl)-3,8-diazabicyclo[3.2.1]octan-8-yl)pyrimidine-5-carboxylate (3)

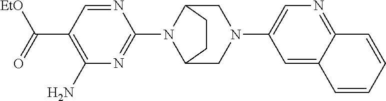 [SMILES: CCOC(=O)c1cnc(N2C3CCC2CN(c2cnc4ccccc4c2)C3)nc1N]

This compound was prepared using the procedure of Example 14 (R=quinolin-3-yl). 1H NMR (400 MHz, CDCl₃) δ 8.75 (s, 1H), 8.66 (s, 1H), 8.02 (d, J=7.9 Hz, 1H), 7.83 (brsm, 1H), 7.64 (d, J=7.8 Hz, 1H), 7.51-7.44 (m, 2H), 7.30 (s, 1H), 5.47 (brsm, 1H), 5.04 (s, 2H), 4.29 (q, J=7.1 Hz, 2H), 3.60 (d, J=11.0 Hz, 2H), 3.22 (d, J=11.0 Hz, 2H), 2.06 (t, J=10.9 Hz, 4H), 1.34 (t, J=7.1 Hz, 3H); ¹³C NMR (101 MHz, CDCl₃) δ 166.4, 163.4, 160.3, 144.6, 142.0, 141.5, 128.1, 127.3, 126.6, 126.4, 116.3, 96.6, 60.4, 53.3, 53.1, 27.7, 14.4. LCMS: 406 [M+H]+; HRMS (ESI) calculated for C₂₂H₂₅N₆O₂+([M+H+]) 405.2034, found: 405.2062. White solid, 68 mg, 56%.

### Example 18: Ethyl 4-amino-2-(8-(pyridin-2-yl)-3,8-diazabicyclo[3.2.1]octan-3-yl)pyrimidine-5-carboxylate (4)

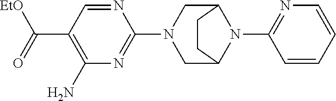 [SMILES: CCOC(=O)c1cnc(N2CC3CCC(C2)N3c2ccccn2)nc1N]

This compound was prepared using the procedure of Example 14 (R=pyridin-2-yl). 1H NMR (400 MHz, CDCl₃) δ 8.60 (s, 1H), 8.17 (d, J=5.7 Hz, 1H), 7.65 (brsm, 1H), 7.47 (t, J=7.8 Hz, 1H), 6.63 (d, J=8.5 Hz, 1H), 6.61-6.56 (m, 1H), 5.33 (brsm, 1H), 4.61 (s, 2H), 4.48 (s, 2H), 4.25 (q, J=7.2 Hz, 2H), 3.25 (d, J=13.0 Hz, 2H), 1.97 (dd, J=8.8, 4.3 Hz, 2H), 1.84-1.80 (m, 2H), 1.31 (t, J=7.1 Hz, 3H); ¹³C NMR (101 MHz, CDCl₃) δ 166.8, 163.2, 163.1, 160.7, 156.9, 148.1, 137.9, 113.1, 96.0, 109.1, 96.1, 60.1, 54.0, 47.6, 27.1, 14.4. LCMS: 355 [M+H]+; HRMS (ESI) calculated for C₁₈H₂₃N₆O₂+([M+H+]) 355.1877, found: 355.1886. White solid, 81 mg, 76%.

### Example 19: Ethyl 4-amino-2-(8-(isoquinolin-3-yl)-3,8-diazabicyclo[3.2.1]octan-3-yl)pyrimidine-5-carboxylate (5)

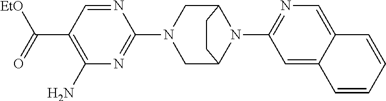 [SMILES: CCOC(=O)c1cnc(N2CC3CCC(C2)N3c2cc3ccccc3cn2)nc1N]

This compound was prepared using the procedure of Example 14 (R=isoquinolin-3-yl). 1H NMR (400 MHz, CDCl₃) δ 8.95 (s, 1H), 8.64 (s, 1H), 7.77 (d, J=8.2 Hz, 1H), 7.56 (d, J=8.3 Hz, 1H), 7.50 (t, J=7.5 Hz, 1H), 7.27-7.23 (m, 1H), 6.81 (s, 1H), 5.40 (brsm, 1H), 4.75 (s, 2H), 4.53 (s, 2H), 4.28 (q, J=7.1 Hz, 2H), 3.37 (s, 2H), 2.16-2.01 (m, 2H), 1.85-1.82 (m, 2H), 1.34 (t, J=7.1 Hz, 3H); ¹³C NMR (101 MHz, CDCl₃) δ 166.5, 162.9, 153.6, 151.7, 148.8, 138.9, 130.6, 127.8, 125.0, 123.4, 123.3, 100.8, 96.3, 60.3, 54.3, 47.6, 27.1, 14.4. LCMS: 405 [M+H]+; HRMS (ESI) calculated for C₂₂H₂₅N₆O₂+([M+H+]) 405.2034, found: 405.2059. White solid, 85 mg, 70%.

### Example 20: Ethyl 4-amino-2-(8-(quinolin-3-yl)-3,8-diazabicyclo[3.2.1]octan-3-yl)pyrimidine-5-carboxylate (6)

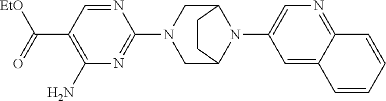 [SMILES: CCOC(=O)c1cnc(N2CC3CCC(C2)N3c2cnc3ccccc3c2)nc1N]

This compound was prepared using the procedure of Example 14 (R=isoquinolin-3-yl). 1H NMR (400 MHz, CDCl₃) δ 8.74 (d, J=2.8 Hz, 1H), 8.62 (s, 1H), 8.02 (s, 1H), 7.72 (brsm, 1H), 7.67-7.61 (m, 1H), 7.52-7.41 (m, 2H), 7.35 (s, 1H), 5.35 (brsm, 1H), 4.50 (s, 2H), 4.44 (s, 2H), 4.28 (q, J=7.1 Hz, 2H), 3.36 (d, J=13.2 Hz, 2H), 2.13-2.05 (m, 2H), 1.90-1.85 (m, 2H), 1.33 (t, J=7.1 Hz, 3H); ¹³C NMR (101 MHz, CDCl₃) δ 166.6, 163.0, 162.7, 160.4, 142.5, 140.3, 129.3, 128.2, 127.5, 127.4, 126.3, 126.1, 96.4, 60.3, 55.1, 46.6, 27.0, 14.4. LCMS: 405 [M+H]+; HRMS (ESI) calculated for C₂₂H₂₅N₆O₂+([M+H+]) 405.2034, found: 405.20942. White solid, 80 mg, 66%.

### Example 21: Ethyl 4-amino-2-(4-(pyridin-2-yl)-1,4-diazepan-1-yl)pyrimidine-5-carboxylate (7)

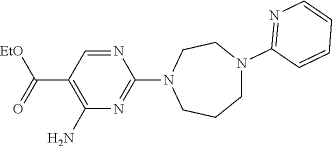 [SMILES: CCOC(=O)c1cnc(N2CCCN(c3ccccn3)CC2)nc1N]

This compound was prepared using the procedure of Example 14 (R=pyridin-2-yl). 1H NMR (400 MHz, CDCl₃) δ 8.65 (s, 1H), 8.17-8.12 (m, 1H), 7.45-7.41 (m, 1H), 6.54-6.51 (m, 2H), 4.28 (q, J=7.1 Hz, 2H), 3.97 (d, J=16.7 Hz, 2H), 3.83 (t, J=5.1 Hz, 2H), 3.66-3.58 (m, 4H), 2.10-2.04 (m, 2H), 1.34 (t, J=7.1 Hz, 3H); ¹³C NMR (101 MHz, CDCl₃) δ 167.1, 162.0, 157.6, 148.4, 137.5, 111.9, 105.8, 96.0, 60.2, 48.3, 46.6, 46.5, 14.5. LCMS: 343 [M+H]+; HRMS (ESI) calculated for C₁₇H₂₃N₆O₂+([M+H+]) 343.1877, found: 343.1870. White solid, 63 mg, 61%.

### Example 22: Ethyl 4-amino-2-(4-(quinolin-3-yl)-1,4-diazepan-1-yl)pyrimidine-5-carboxylate (8)

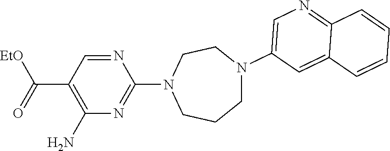 [SMILES: CCOC(=O)c1cnc(N2CCCN(c3cnc4ccccc4c3)CC2)nc1N]

This compound was prepared using the procedure of Example 14 (R=quinolin-3-yl). 1H NMR (400 MHz, CDCl₃) δ 8.89 (s, 1H), 8.64 (s, 1H), 7.83 (brsm, 1H), 7.73 (d, J=8.1 Hz, 1H), 7.53 (d, J=8.4 Hz, 1H), 7.46 (ddd, J=8.1, 6.6, 1.2 Hz, 1H), 7.18 (ddd, J=8.0, 6.6, 1.2 Hz, 1H), 6.65 (s, 1H), 5.61 (brsm, 1H), 4.28 (q, J=7.1 Hz, 2H), 4.12-3.96 (m, 4H), 3.81-3.60 (m, 4H), 2.17 (s, 2H), 1.33 (t, J=7.1 Hz, 3H); ¹³C NMR (101 MHz, CDCl₃) δ 166.3, 162.9, 159.8, 153.9, 151.1, 139.3, 130.5, 127.7, 124.9, 122.5, 96.8, 96.2, 60.5, 49.1, 48.5, 47.0, 46.8, 24.4, 24.1, 14.3. LCMS: 393 [M+H]⁺; HRMS (ESI) calculated for C₂₁H₂₅N₆O₂+([M+H+]) 393.2034, found: 393.2033. White solid, 89 mg, 76%.

### Example 23: Ethyl 4-amino-2-(4-(pyridin-2-yl)piperazin-1-yl)pyrimidine-5-carboxylate (9)

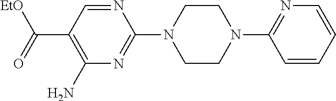 [SMILES: CCOC(=O)c1cnc(N2CCN(c3ccccn3)CC2)nc1N]

This compound was prepared using the procedure of Example 14 (R=pyridin-2-yl). 1H NMR (400 MHz, DMSO-d6) δ 8.52 (s, 1H), 8.12 (ddd, J=4.9, 2.0, 0.8 Hz, 1H), 7.55 (ddd, J=8.9, 7.1, 2.0 Hz, 1H), 7.47 (s, 1H), 7.41 (s, 1H), 6.86 (d, J=8.6 Hz, 1H), 6.66 (dd, J=6.7, 4.5 Hz, 1H), 4.21 (q, J=7.1 Hz, 2H), 1.26 (t, J=7.1 Hz, 3H); ¹³C NMR (101 MHz, DMSO-d6) δ 166.1, 162.9, 161.5, 160.6, 158.9, 147.7, 137.8, 113.4, 107.4, 95.1, 59.9, 44.5, 43.0, 14.4. LCMS: 329 [M+H]+; HRMS (ESI) calculated for C₁₆H₂₁N₆O₂+([M+H+]) 329.1721, found: 329.1745. White solid, 85 mg, 51%.

### Example 24: Ethyl 4-amino-2-(4-(benzo[d]oxazol-2-yl)piperazin-1-yl)pyrimidine-5-carboxylate (10)

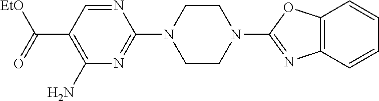 [SMILES: CCOC(=O)c1cnc(N2CCN(c3nc4ccccc4o3)CC2)nc1N]

This compound was prepared using the procedure of Example 14 (R=benzo[d]oxaol-2-yl). 1H NMR (400 MHz, CDCl₃) δ 8.67 (s, 1H), 7.76 (brsm, 1H), 7.38 (dd, J=7.8, 1.2 Hz, 1H), 7.27 (dd, J=9.3, 1.8 Hz, 1H), 7.18 (td, J=7.7, 1.1 Hz, 1H), 7.04 (td, J=7.7, 1.2 Hz, 1H), 5.33 (brsm, 1H), 4.30 (q, J=7.1 Hz, 2H), 4.02 (t, J=5.2 Hz, 4H), 3.80-3.70 (m, 4H), 1.35 (t, J=7.1 Hz, 3H); ¹³C NMR (101 MHz, CDCl₃) δ 166.7, 163.4, 162.2, 148.9, 143.1, 124.3, 121.1, 116.6, 109.0, 96.7, 60.4, 45.6, 43.3, 14.5. LCMS: 369 [M+H]+; HRMS (ESI) calculated for C₁₈H₂₁N₆O₃+([M+H+]) 369.1670, found: 369.1678. White solid, 84 mg, 77%.

### Example 25: Ethyl 4-amino-2-(4-(benzo[d]thiazol-2-yl)piperazin-1-yl)pyrimidine-5-carboxylate (11)

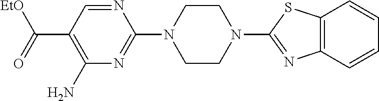 [SMILES: CCOC(=O)c1cnc(N2CCN(c3nc4ccccc4s3)CC2)nc1N]

This compound was prepared using the procedure of Example 14 (R=benzo[d]thiazol-2-yl). 1H NMR (400 MHz, DMSO-d6) δ 8.54 (s, 1H), 7.78 (dd, J=8.0, 1.2 Hz, 1H), 7.56-7.44 (m, 3H), 7.29 (td, J=8.2, 1.3 Hz, 1H), 7.08 (td, J=7.6, 1.2 Hz, 1H), 4.22 (q, J=7.1 Hz, 2H), 3.95 (t, J=5.2 Hz, 4H), 3.65 (t, J=5.2 Hz, 4H), 1.28 (t, J=7.1 Hz, 3H); ¹³C NMR (101 MHz, DMSO-d6) δ 168.1, 165.9, 162.8, 161.3, 160.5, 152.3, 130.4, 126.0, 121.3, 121.2, 118.6, 95.2, 59.7, 47.7, 42.5, 14.2. LCMS: 385 [M+H]+; HRMS (ESI) calculated for C₁₈H₂₁N₆O₂S+([M+H+]) 385.1441, found: 385.1452. White solid, 86 mg, 76%.

### Example 26: Ethyl 4-amino-2-(3-(benzo[d]thiazol-2-yl)-3,8-diazabicyclo[3.2.1]octan-8-yl)pyrimidine-5-carboxylate (12)

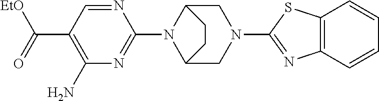 [SMILES: CCOC(=O)c1cnc(N2C3CCC2CN(c2nc4ccccc4s2)C3)nc1N]

This compound was prepared using the procedure of Example 14 (R=benzo[d]thiazol-2-yl). 1H NMR (400 MHz, DMSO-d6) δ 8.55 (s, 1H), 7.75 (dd, J=7.9, 1.2 Hz, 1H), 7.51 (s, 2H), 7.45 (d, J=7.5 Hz, 1H), 7.27 (td, J=7.7, 1.3 Hz, 1H), 7.06 (td, J=7.6, 1.2 Hz, 1H), 4.88 (d, J=22.3 Hz, 2H), 4.22 (q, J=7.1 Hz, 2H), 3.85 (d, J=12.0 Hz, 2H), 3.36 (d, J=12.0 Hz, 2H), 1.98-1.94 (m, 2H), 1.81 (q, J=6.7 Hz, 2H), 1.27 (t, J=7.1 Hz, 3H); ¹³C NMR (101 MHz, DMSO-d6) δ 169.0, 165.9, 163.1, 160.8, 159.9, 152.3, 130.0, 126.0, 121.1, 121.1, 118.5, 95.6, 59.7, 52.4, 26.7, 14.2. LCMS: 411 [M+H]+; HRMS (ESI) calculated for C₂₀H₂₃N₆O₂S+([M+H+]) 411.1598, found: 411.1605. White solid, 87 mg, 71%.

### Example 27: Ethyl 4-amino-2-(4-(isoquinolin-3-yl)piperazin-1-yl)pyrimidine-5-carboxylate (13)

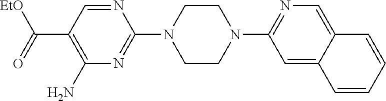 [SMILES: CCOC(=O)c1cnc(N2CCN(c3cc4ccccc4cn3)CC2)nc1N]

This compound was prepared using the procedure of Example 14 (R=isoquinolin-3-yl). 1H NMR (400 MHz, CDCl₃) δ 8.97 (s, 1H), 8.69 (s, 1H), 7.85-7.76 (m, 1H), 7.55-7.51 (m, 1H), 7.32-7.28 (m, 1H), 6.82 (s, 1H), 4.31 (q, J=7.1 Hz, 2H), 4.10 (s, 4H), 3.68 (t, J=5.3 Hz, 4H), 1.36 (t, J=7.1 Hz, 4H); ¹³C NMR (101 MHz, CDCl₃) δ 163.0, 156.1, 151.1, 149.0, 138.8, 131.3, 130.6, 127.7, 125.4, 123.85, 123.79, 122.8, 99.6, 60.7, 46.2, 44.0, 14.3. LCMS: 379 [M+H]+; HRMS (ESI) calculated for C₂₀H₂₃N₆O₂+([M+H+]) 379.1877, found: 379.1911. White solid, 91 mg, 80%.

### Example 28: Ethyl 4-amino-2-(4-(quinolin-3-yl)piperazin-1-yl)pyrimidine-5-carboxylate (14)

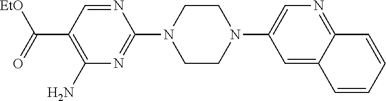 [SMILES: CCOC(=O)c1cnc(N2CCN(c3cnc4ccccc4c3)CC2)nc1N]

This compound was prepared using the procedure of Example 14 (R=quinolin-3-yl). 1H NMR (400 MHz, CDCl₃) δ 8.83 (d, J=2.8 Hz, 1H), 8.68 (s, 1H), 8.14 (s, 1H), 7.84 (brsm, 1H), 7.76-7.69 (m, 1H), 7.61-7.47 (m, 3H), 5.46 (brsm, 1H), 4.31 (q, J=7.1, 2H), 4.13 (s, 4H), 3.37 (d, J=5.3 Hz, 4H), 1.36 (t, J=7.1 Hz, 3H); ¹³C NMR (101 MHz, CDCl₃) δ 166.4, 163.2, 144.6, 142.6, 129.0, 127.9, 127.8, 127.6, 127.4, 126.7, 96.6, 60.5, 49.0, 43.6, 14.4. LCMS: 379 [M+H]⁺; HRMS (ESI) calculated for C₂₀H₂₃N₆O₂+([M+H+]) 379.1877, found: 379.1911. White solid, 88 mg, 78%.

### Example 29: Ethyl 4-amino-2-(4-(quinolin-2-yl)piperazin-1-yl)pyrimidine-5-carboxylate (15)

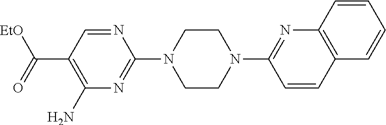 [SMILES: CCOC(=O)c1cnc(N2CCN(c3ccc4ccccc4n3)CC2)nc1N]

This compound was prepared using the procedure of Example 14 (R=quinolin-2-yl). 1H NMR (400 MHz, CDCl₃) δ 8.67 (s, 1H), 7.95 (d, J=9.1 Hz, 1H), 7.74 (brsm, 1H), 7.65-7.52 (m, 2H), 7.26 (d, J=3.0 Hz, 1H), 7.01 (d, J=9.2 Hz, 1H), 5.29 (brsm, 1H), 4.29 (q, J=7.1 Hz, 2H), 4.11-3.97 (m, 4H), 3.86 (s, 4H), 1.35 (t, J=7.1 Hz, 3H); ¹³C NMR (101 MHz, CDCl₃) δ 166.9, 163.4, 161.9, 161.0, 130.1, 127.4, 123.1, 109.8, 96.4, 60.3, 45.5, 43.6, 14.5. LCMS: 379 [M+H]⁺; HRMS (ESI) calculated for C₂₀H₂₃N₆O₂+([M+H+]) 379.1877, found: 379.1905. Light-yellow solid, 93 mg, 82%.

### Example 30: Ethyl 4-amino-2-(4-(quinoxalin-2-yl)piperazin-1-yl)pyrimidine-5-carboxylate (16)

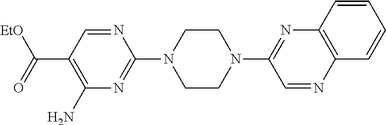 [SMILES: CCOC(=O)c1cnc(N2CCN(c3cnc4ccccc4n3)CC2)nc1N]

This compound was prepared using the procedure of Example 14 (R=quinoxalin-2-yl). 1H NMR (400 MHz, DMSO-d6) δ 8.75 (d, J=1.8 Hz, 1H), 8.63 (d, J=1.9 Hz, 1H), 8.55 (s, 1H), 7.91 (d, J=9.3 Hz, 1H), 7.76 (dd, J=9.4, 2.7 Hz, 1H), 7.48 (d, J=7.5 Hz, 2H), 7.28 (d, J=2.7 Hz, 1H), 4.22 (q, J=7.1 Hz, 2H), 3.98 (t, J=5.0 Hz, 4H), 3.47 (t, J=5.2 Hz, 4H), 1.28 (t, J=7.0 Hz, 3H); ¹³C NMR (101 MHz, DMSO-d6) δ 165.9, 162.8, 161.3, 160.5, 151.4, 145.5, 144.1, 141.8, 137.4, 129.4, 122.3, 108.5, 95.0, 59.7, 47.4, 42.8, 14.2. LCMS: 380 [M+H]+; HRMS (ESI) calculated for C₁₉H₂₂N₇O₂+([M+H+]) 380.1829, found: 380.1861. Yellow solid, 90 mg, 80%.

### Example 31: Ethyl 4-amino-2-(4-(4-methoxyphenyl)piperazin-1-yl)pyrimidine-5-carboxylate (17)

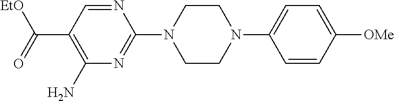 [SMILES: CCOC(=O)c1cnc(N2CCN(c3ccc(OC)cc3)CC2)nc1N]

This compound was prepared using the procedure of Example 14 (R=4-methoxyphenyl). 1H NMR (400 MHz, DMSO-d6) δ 8.52 (s, 1H), 7.43 (d, J=28.6 Hz, 2H), 7.00-6.86 (m, 4H), 4.22 (q, J=7.1 Hz, 2H), 3.91 (t, J=5.0 Hz, 4H), 3.80 (s, 3H), 2.97 (t, J=5.0 Hz, 4H), 1.27 (t, J=7.1 Hz, 3H); ¹³C NMR (101 MHz, DMSO-d6) δ 165.9, 162.8, 161.3, 160.4, 152.1, 141.0, 122.8, 120.8, 118.4, 111.9, 94.8, 59.6, 55.4, 50.1, 43.4, 14.2. LCMS: 358 [M+H]+; HRMS (ESI) calculated for C₁₈H₂₄N₅O₃+([M+H+]) 358.1874, found: 358.1877. White solid, 87 mg, 82%.

### Example 32: 4-Amino-N-(4-methoxyphenyl)-2-(3-(pyridin-2-yl)-3,8-diazabicyclo[3.2.1]octan-8-yl)pyrimidine-5-carboxamide (18)

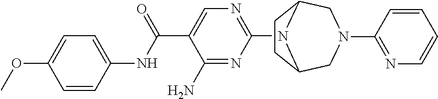 [SMILES: COc1ccc(NC(=O)c2cnc(N3C4CCC3CN(c3ccccn3)C4)nc2N)cc1]

This compound was prepared using the procedure of Example 14 (R=pyridin-2-yl). 1H NMR (400 MHz, DMSO-d6) δ 9.79 (s, 1H), 8.63 (s, 1H), 8.13 (dd, J=5.2, 1.9 Hz, 1H), 7.56 (d, J=8.9 Hz, 3H), 6.93 (d, J=9.0 Hz, 2H), 6.78 (d, J=8.7 Hz, 1H), 6.67 (dd, J=7.0, 4.9 Hz, 1H), 4.90 (brsm, 2H), 4.05 (dd, J=11.7, 2.5 Hz, 2H), 3.77 (s, 3H), 3.01 (dd, J=12.1, 2.2 Hz, 2H), 1.94 (dd, J=8.4, 4.2 Hz, 2H), 1.81 (t, J=6.4 Hz, 2H); ¹³C NMR (101 MHz, DMSO-d6) δ 165.8, 163.6, 160.3, 160.2, 158.2, 155.9, 147.9, 137.8, 132.5, 122.8, 114.1, 113.4, 107.1, 100.0, 55.6, 53.0, 50.0, 27.5. LCMS: 432 [M+H]+; HRMS (ESI) calculated for C₂₃H₂₆N₇O₂+([M+H+]) 432.2142, found: 432.2149. White solid, 36 mg, 42% for 2 steps.

### Example 33: 4-Amino-2-(3-(isoquinolin-3-yl)-3,8-diazabicyclo[3.2.1]octan-8-yl)-N-(4-methoxyphenyl)pyrimidine-5-carboxamide (19)

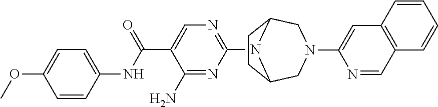 [SMILES: COc1ccc(NC(=O)c2cnc(N3C4CCC3CN(c3cc5ccccc5cn3)C4)nc2N)cc1]

This compound was prepared using the procedure of Example 14 (R=isoquinolin-3-yl). 1H NMR (400 MHz, DMSO-d6) δ 9.79 (s, 1H), 9.00 (s, 1H), 8.64 (s, 1H), 7.99 (s, 1H), 7.90 (d, J=8.2 Hz, 1H), 7.68 (d, J=8.5 Hz, 1H), 7.59-7.55 (m, 3H), 7.30 (t, J=7.6 Hz, 1H), 6.94 (s, 2H), 4.96 (brsm, 2H), 4.15 (d, J=11.9 Hz, 2H), 3.77 (s, 3H), 3.08 (d, J=11.8 Hz, 2H), 1.97 (s, 2H), 1.87 (d, J=7.7 Hz, 2H); ¹³C NMR (101 MHz, DMSO-d6) δ 165.3, 163.1, 162.3, 159.8, 157.7, 156.8, 155.4, 150.7, 138.3, 132.0, 130.3, 127.5, 125.0, 122.9, 122.3, 113.7, 99.5, 97.7, 55.2, 52.6, 50.3, 27.1. LCMS: 482 [M+H]+; HRMS (ESI) calculated for C₂₇H₂₈N₇O₂+([M+H+]) 482.2299, found: 482.2288. Light-yellow solid, 48 mg, 50% for 2 steps.

### Example 34: 4-Amino-N-(4-methoxyphenyl)-2-(3-(quinolin-3-yl)-3,8-diazabicyclo[3.2.1]octan-8-yl)pyrimidine-5-carboxamide (20)

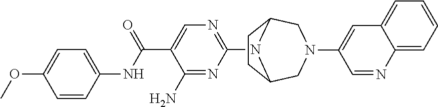 [SMILES: COc1ccc(NC(=O)c2cnc(N3C4CCC3CN(c3cnc5ccccc5c3)C4)nc2N)cc1]

This compound was prepared using the procedure of Example 14 (R=quinolin-3-yl). 1H NMR (400 MHz, DMSO-d6) δ 9.80 (s, 1H), 8.90 (d, J=2.8 Hz, 1H), 8.65 (s, 1H), 7.94-7.86 (m, 1H), 7.85-7.75 (m, 1H), 7.56 (d, J=9.0 Hz, 2H), 7.52-7.47 (m, 3H), 6.93 (d, J=9.0 Hz, 2H), 4.95 (brsm, 2H), 3.80 (dd, J=11.4, 2.4 Hz, 2H), 3.77 (s, 3H), 3.07 (dd, J=11.3, 2.2 Hz, 3H), 2.05-1.96 (m, 4H); ¹³C NMR (101 MHz, DMSO-d6) δ 165.8, 163.6, 160.3, 158.2, 155.9, 145.1, 143.4, 142.2, 132.5, 129.1, 128.8, 127.2, 127.0, 126.0, 122.8, 114.9, 114.1, 100.1, 55.6, 53.1, 52.6, 27.7. LCMS: 482 [M+H]+; HRMS (ESI) calculated for C₂₇H₂₈N₇O₂+([M+H+]) 482.2299, found: 482.2312. Light-yellow solid, 41 mg, 43% for 2 steps.

### Example 35: 4-Amino-2-(8-(isoquinolin-3-yl)-3,8-diazabicyclo[3.2.1]octan-3-yl)-N-(4-methoxyphenyl)pyrimidine-5-carboxamide (21)

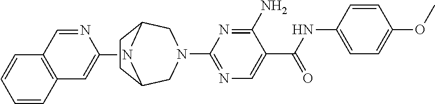 [SMILES: COc1ccc(NC(=O)c2cnc(N3CC4CCC(C3)N4c3cc4ccccc4cn3)nc2N)cc1]

This compound was prepared using the procedure of Example 14 (R=isoquinolin-3-yl). 1H NMR (400 MHz, DMSO-d6) δ 9.73 (s, 1H), 9.00 (s, 1H), 8.57 (s, 1H), 7.87 (dd, J=8.3, 1.1 Hz, 1H), 7.64 (dd, J=8.5, 1.1 Hz, 1H), 7.55-7.50 (m, 3H), 7.26 (ddd, J=8.1, 6.7, 1.1 Hz, 1H), 7.09 (s, 1H), 6.89 (d, J=9.1 Hz, 2H), 4.77 (brsm, 2H), 4.47 (d, J=12.8 Hz, 2H), 3.73 (s, 3H), 3.20 (dd, J=13.1, 2.0 Hz, 2H), 1.94 (dd, J=8.4, 4.1 Hz, 2H), 1.72 (t, J=6.7 Hz, 2H); ¹³C NMR (101 MHz, DMSO-d6) δ 165.9, 163.1, 162.7, 157.7, 155.8, 154.2, 152.1, 138.9, 132.6, 130.9, 128.1, 125.3, 123.4, 123.3, 122.7, 114.1, 100.5, 99.5, 55.6, 54.0, 47.4, 27.2. LCMS: 482 [M+H]+; HRMS (ESI) calculated for C₂₇H₂₈N₇O₂+([M+H+]) 482.2299, found: 482.2278. Light-yellow solid, 51 mg, 53% for 2 steps.

### Example 36: 4-Amino-N-(4-methoxyphenyl)-2-(8-(quinolin-3-yl)-3,8-diazabicyclo[3.2.1]octan-3-yl)pyrimidine-5-carboxamide (22)

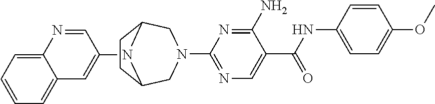 [SMILES: COc1ccc(NC(=O)c2cnc(N3CC4CCC(C3)N4c3cnc4ccccc4c3)nc2N)cc1]

This compound was prepared using the procedure of Example 14 (R=quinolin-3-yl). 1H NMR (400 MHz, DMSO-d6) δ 9.73 (s, 1H), 8.87 (d, J=2.9 Hz, 1H), 8.57 (s, 1H), 7.85 (dd, J=7.7, 1.8 Hz, 1H), 7.76-7.71 (m, 1H), 7.61 (d, J=2.8 Hz, 1H), 7.53 (d, J=9.0 Hz, 2H), 7.47-7.43 (m, 2H), 6.89 (d, J=9.0 Hz, 2H), 4.64 (s, 2H), 4.42 (d, J=13.0 Hz, 2H), 3.73 (s, 3H), 3.24 (d, J=12.9 Hz, 2H), 1.99 (d, J=11.5 Hz, 3H), 1.73 (d, J=7.4 Hz, 2H); ¹³C NMR (101 MHz, DMSO-d6) δ 165.8, 163.1, 162.7, 157.7, 155.8, 144.2, 141.9, 140.8, 132.6, 129.6, 128.9, 127.2, 126.7, 125.7, 122.7, 116.0, 114.1, 99.6, 55.6, 54.6, 46.6, 27.1. LCMS: 482 [M+H]+; HRMS (ESI) calculated for C₂₇H₂₈N₇O₂+([M+H+]) 482.2299, found: 482.2299. Light-yellow solid, 42 mg, 44% for 2 steps.

### Example 37: 4-Amino-N-(4-methoxyphenyl)-2-(4-(pyridin-2-yl)-1,4-diazepan-1-yl)pyrimidine-5-carboxamide (23)

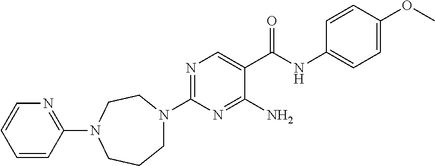 [SMILES: COc1ccc(NC(=O)c2cnc(N3CCCN(c4ccccn4)CC3)nc2N)cc1]

This compound was prepared using the procedure of Example 14 (R=pyridin-2-yl). 1H NMR (400 MHz, CDCl₃) δ 9.38 (brsm, 1H), 8.86 (s, 1H), 8.15 (d, J=5.4 Hz, 1H), 7.63-7.52 (m, 3H), 6.84 (d, J=9.1 Hz, 2H), 6.65 (dd, J=11.6, 7.4 Hz, 2H), 4.04 (s, 4H), 3.78 (s, 3H), 3.70 (s, 4H), 2.08 (t, J=6.7 Hz, 2H); ¹³C NMR (101 MHz, CDCl₃) δ 163.1, 156.7, 130.8, 125.6, 123.3, 123.2, 114.8, 114.0, 112.2, 55.5, 48.3, 47.8, 29.7. LCMS: 420 [M+H]+; HRMS (ESI) calculated for C₂₂H₂₆N₇O₂+([M+H+]) 420.2142, found: 420.2153. White solid, 33 mg, 39% for 2 steps.

### Example 38: 4-Amino-2-(4-(isoquinolin-3-yl)-1,4-diazepan-1-yl)-N-(4-methoxyphenyl)pyrimidine-5-carboxamide (24)

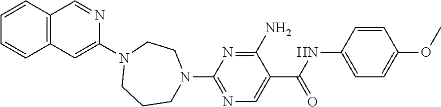 [SMILES: COc1ccc(NC(=O)c2cnc(N3CCCN(c4cc5ccccc5cn4)CC3)nc2N)cc1]

This compound was prepared using the procedure of Example 14 (R=isoquinolin-3-yl). 1H NMR (400 MHz, DMSO-d6) δ 9.69 (s, 1H), 8.92 (s, 1H), 8.54 (s, 1H), 7.80 (d, J=8.2 Hz, 1H), 7.61 (d, J=8.3 Hz, 1H), 7.51 (d, J=9.1 Hz, 2H), 7.49-7.44 (m, 1H), 7.17 (ddd, J=8.0, 6.7, 1.1 Hz, 1H), 6.88 (d, J=9.1 Hz, 2H), 6.84 (s, 1H), 3.96 (d, J=18.3 Hz, 2H), 3.88 (s, 2H), 3.72 (s, 3H), 3.70 (d, J=5-7 Hz, 2H), 3.63 (s, 2H), 2.08-1.95 (m, 2H); ¹³C NMR (101 MHz, DMSO-d6) δ 165.9, 163.4, 161.3, 158.0, 155.8, 154.7, 151.6, 139.2, 132.6, 130.6, 128.0, 125.2, 122.7, 122.5, 114.1, 99.3, 96.4, 55.6, 47.3, 46.6, 46.1, 25.1. LCMS: 470 [M+H]+; HRMS (ESI) calculated for C₂₆H₂₈N₇O₂+([M+H+]) 470.2299, found: 470.2318. Light-yellow solid, 36 mg, 39% for 2 steps.

### Example 39: 4-Amino-N-(4-methoxyphenyl)-2-(4-(quinolin-3-yl)-1,4-diazepan-1-yl)pyrimidine-5-carboxamide (25)

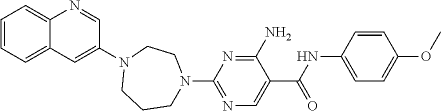 [SMILES: COc1ccc(NC(=O)c2cnc(N3CCCN(c4cnc5ccccc5c4)CC3)nc2N)cc1]

This compound was prepared using the procedure of Example 14 (R=quinolin-3-yl). 1H NMR (400 MHz, DMSO-d6) δ 9.67 (s, 1H), 8.75 (d, J=2.9 Hz, 1H), 8.53 (s, 1H), 7.79 (d, J=8.0 Hz, 1H), 7.70 (d, J=8.0 Hz, 1H), 7.50 (d, J=9.0 Hz, 2H), 7.42-7.33 (m, 3H), 6.88 (d, J=9.0 Hz, 2H), 4.00 (d, J=14.9 Hz, 2H), 3.79 (t, J=5.3 Hz, 2H), 3.72 (s, 3H), 3.69-3.59 (m, 4H), 2.07-1.97 (m, 2H); ¹³C NMR (101 MHz, DMSO-d6) δ 165.4, 160.7, 157.5, 155.3, 140.4, 132.1, 129.3, 128.3, 126.5, 126.0, 124.3, 122.2, 113.6, 110.9, 98.9, 55.1, 47.5, 45.8, 28.8. LCMS: 470 [M+H]+; HRMS (ESI) calculated for C₂₆H₂₈N₇O₂+([M+H+]) 470.2299, found: 470.2297. Light-yellow solid, 38 mg, 40% for 2 steps.

### Example 40: 4-Amino-2-(4-(benzo[d]oxazol-2-yl)piperazin-1-yl)-N-(4-methoxyphenyl)pyrimidine-5-carboxamide (26)

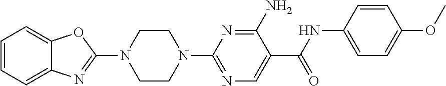 [SMILES: COc1ccc(NC(=O)c2cnc(N3CCN(c4nc5ccccc5o4)CC3)nc2N)cc1]

This compound was prepared using the procedure of Example 14 (R=benzo[d]oxazol-2-yl). 1H NMR (400 MHz, DMSO-d6) δ 9.77 (s, 1H), 8.62 (s, 1H), 7.54 (d, J=9.1 Hz, 2H), 7.42 (d, J=7.7 Hz, 1H), 7.31 (d, J=7.7 Hz, 1H), 7.17 (td, J=7.7, 1.1 Hz, 1H), 7.04 (td, J=7.7, 1.1 Hz, 1H), 6.90 (d, J=9.1 Hz, 2H), 3.94 (t, J=5.2 Hz, 4H), 3.73 (s, 3H), 3.68 (t, J=5.2 Hz, 4H); ¹³C NMR (101 MHz, DMSO-d6) δ 165.3, 162.9, 161.8, 160.8, 157.4, 155.4, 148.3, 142.9, 132.0, 124.0, 122.3, 120.6, 115.9, 113.7, 108.9, 99.3, 55.2, 45.1, 42.5. LCMS: 446 [M+H]+; HRMS (ESI) calculated for C₂₃H₂₄N₇O₃+([M+H+]) 446.1935, found: 446.1941. White solid, 39 mg, 44% for 2 steps.

### Example 41: 4-Amino-2-(4-(benzo[d]thiazol-2-yl)piperazin-1-yl)-N-(4-methoxyphenyl)pyrimidine-5-carboxamide (27)

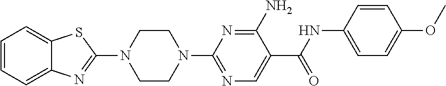 [SMILES: COc1ccc(NC(=O)c2cnc(N3CCN(c4nc5ccccc5s4)CC3)nc2N)cc1]

This compound was prepared using the procedure of Example 14 (R=benzo[d]thiazol-2-yl). 1H NMR (400 MHz, DMSO-d6) δ 9.77 (d, J=3.9 Hz, 1H), 8.62 (d, J=4.4 Hz, 1H), 7.79 (s, 1H), 7.56-7.46 (m, 3H), 7.29 (d, J=6.9 Hz, 1H), 7.09 (d, J=7.0 Hz, 1H), 6.90 (s, 2H), 3.94 (s, 4H), 3.73 (s, 3H), 3.66 (s, 4H); ¹³C NMR (101 MHz, DMSO-d6) δ 168.2, 165.3, 162.9, 160.8, 157.5, 155.4, 152.4, 132.0, 130.4, 126.0, 122.3, 121.4, 121.2, 118.7, 113.7, 99.3, 55.2, 47.8, 42.6. LCMS: 462 [M+H]+; HRMS (ESI) calculated for C₂₃H₂₄N₇O₂S+([M+H+]) 462.1707, found: 4621705. White solid, 48 mg, 51% for 2 steps.

### Example 42: 4-Amino-N-(4-methoxyphenyl)-2-(4-(pyridin-2-yl)piperazin-1-yl)pyrimidine-5-carboxamide (28)

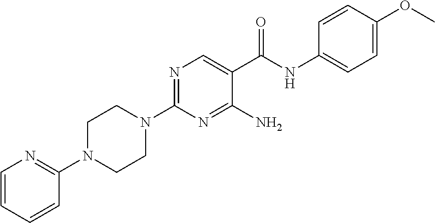 [SMILES: COc1ccc(NC(=O)c2cnc(N3CCN(c4ccccn4)CC3)nc2N)cc1]

This compound was prepared using the procedure of Example 14 (R=pyridin-2-yl). 1H NMR (400 MHz, DMSO-d6) δ 9.75 (s, 1H), 8.61 (s, 1H), 8.13 (d, J=4.7 Hz, 1H), 7.64-7.45 (m, 4H), 6.95-6.85 (m, 3H), 6.66 (t, J=5.9 Hz, 1H), 3.87 (t, J=5.2 Hz, 4H), 3.73 (t, J=1.5 Hz, 3H), 3.56 (t, J=5.2 Hz, 4H); ¹³C NMR (101 MHz, DMSO-d6) δ 165.4, 162.9, 160.9, 158.9, 157.4, 155.4, 147.6, 137.6, 132.0, 122.3, 113.7, 113.2, 107.2, 99.0, 55.2, 44.5, 42.9. LCMS: 406 [M+H]+; HRMS (ESI) calculated for C₂₁H₂₄N₇O₂+([M+H+]) 406.1986, found: 406.2014. White solid, 35 mg, 43%.

### Example 43: 4-Amino-2-(4-(isoquinolin-3-yl)piperazin-1-yl)-N-(4-methoxyphenyl)pyrimidine-5-carboxamide (29)

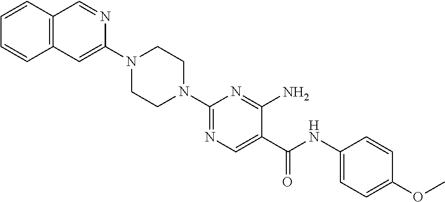 [SMILES: COc1ccc(NC(=O)c2cnc(N3CCN(c4cc5ccccc5cn4)CC3)nc2N)cc1]

This compound was prepared using the procedure of Example 14 (R=isoquinolin-3-yl). 1H NMR (400 MHz, DMSO-d6) δ 9.76 (s, 1H), 9.01 (s, 1H), 8.63 (s, 1H), 7.89 (d, J=8.2 Hz, 1H), 7.69 (d, J=8.4 Hz, 1H), 7.58-7.52 (m, 3H), 7.29 (t, J=7.5 Hz, 1H), 7.04 (s, 1H), 6.90 (d, J=9.0 Hz, 2H), 3.94 (t, J=5.2 Hz, 4H), 3.73 (s, 3H), 3.63 (t, J=5.2 Hz, 4H); ¹³C NMR (101 MHz, DMSO-d6) δ 165.8, 163.4, 161.4, 157.9, 156.5, 155.9, 151.4, 138.8, 132.5, 130.9, 128.0, 125.6, 123.7, 123.5, 122.8, 114.1, 99.5, 99.0, 55.6, 45.8, 43.4. LCMS: 456 [M+H]+; HRMS (ESI) calculated for C₂₅H₂₆N₇O₂+([M+H+]456.2142, found: 456.2133. Light-yellow solid, 44 mg, 48% for 2 steps.

### Example 44: 4-Amino-N-(4-methoxyphenyl)-2-(4-(quinolin-3-yl)piperazin-1-yl)pyrimidine-5-carboxamide (30)

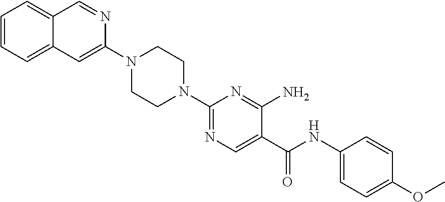 [SMILES: COc1ccc(NC(=O)c2cnc(N3CCN(c4cc5ccccc5cn4)CC3)nc2N)cc1]

This compound was prepared using the procedure of Example 14 (R=quinolin-3-yl). 1H NMR (400 MHz, DMSO-d6) δ 9.77 (s, 1H), 8.91 (d, J=2.8 Hz, 1H), 8.62 (s, 1H), 7.91-7.85 (m, 1H), 7.82-7.76 (m, 1H), 7.59 (d, J=2.9 Hz, 1H), 7.53 (d, J=9.0 Hz, 2H), 7.51-7.48 (m, 2H), 6.89 (d, J=9.0 Hz, 2H), 3.98 (t, J=5.0 Hz, 4H), 3.36 (t, J=5.0 Hz, 4H); ¹³C NMR (101 MHz, DMSO-d6) δ 165.8, 163.4, 162.9, 161.3, 157.9, 155.9, 145.1, 145.0, 142.6, 132.4, 129.1, 128.8, 127.3, 127.2, 126.6, 122.8, 118.6, 116.5, 114.2, 99.6, 55.6, 48.7, 43.4. LCMS: 456 [M+H]+; HRMS (ESI) calculated for C₂₅H₂₆N₇O₂+([M+H+]456.2142, found: 456.2152. Brown solid, 51 mg, 56% for 2 steps.

### Example 45: 4-Amino-N-(4-methoxyphenyl)-2-(4-(quinolin-2-yl)piperazin-1-yl)pyrimidine-5-carboxamide (Compound 31)

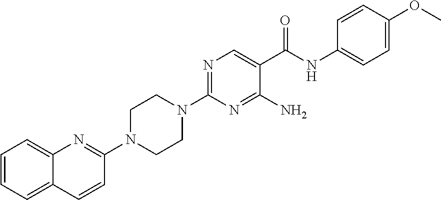 [SMILES: COc1ccc(NC(=O)c2cnc(N3CCN(c4ccc5ccccc5n4)CC3)nc2N)cc1]

This compound was prepared using the procedure of Example 14 (R=quinolin-2-yl). 1H NMR (400 MHz, DMSO-d6) δ 9.75 (s, 1H), 8.63 (s, 1H), 8.08 (d, J=9.1 Hz, 1H), 7.72 (dd, J=8.2, 1.4 Hz, 1H), 7.61-7.57 (m, 1H), 7.56-7.51 (m, 3H), 7.30 (d, J=9.0 Hz, 1H), 7.24 (ddd, J=8.1, 6.7, 1.3 Hz, 1H), 6.90 (d, J=9.0 Hz, 2H), 3.92 (dd, J=6.9, 3.7 Hz, 4H), 3.78 (dd, J=6.8, 3.9 Hz, 4H), 3.73 (s, 3H); ¹³C NMR (101 MHz, DMSO-d6) δ 165.8, 163.4, 161.4, 157.9, 157.5, 155.9, 147.7, 137.9, 132.5, 129.9, 127.9, 126.5, 123.3, 122.7, 122.6, 114.1, 110.7, 99.5, 55.6, 44.9, 43.5. LCMS: 456 [M+H]⁺; HRMS (ESI) calculated for C₂₅H₂₆N₇O₂+([M+H+]) 456.2142, found: 456.2165. Light-yellow solid, 46 mg, 50% for 2 steps.

### Example 46: 4-Amino-N-(4-methoxyphenyl)-2-(4-(quinoxalin-2-yl)piperazin-1-yl)pyrimidine-5-carboxamide (32)

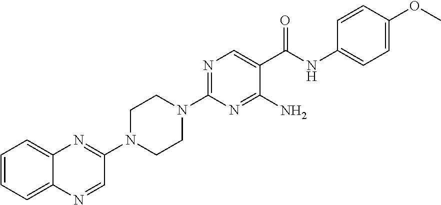 [SMILES: COc1ccc(NC(=O)c2cnc(N3CCN(c4cnc5ccccc5n4)CC3)nc2N)cc1]

This compound was prepared using the procedure of Example 14 (R=quinoxalin-2-yl). 1H NMR (400 MHz, DMSO-d6) δ 9.76 (s, 1H), 8.76 (d, J=1.9 Hz, 1H), 8.65-8.60 (m, 2H), 7.92 (d, J=9.3 Hz, 1H), 7.78 (dd, J=9.3, 2.7 Hz, 1H), 7.54 (d, J=8.9 Hz, 2H), 7.29 (d, J=2.7 Hz, 1H), 6.90 (d, J=8.9 Hz, 2H), 3.97 (t, J=5.2 Hz, 4H), 3.73 (s, 3H), 3.48 (t, J=5.2 Hz, 4H); ¹³C NMR (101 MHz, DMSO-d6) δ 165.3, 162.9, 160.8, 157.5, 155.4, 151.5, 145.5, 144.1, 141.9, 137.4, 132.0, 129.5, 122.4, 122.3, 113.7, 108.6, 99.1, 55.2, 47.5, 42.8. LCMS: 462 [M+H]+; HRMS (ESI) calculated for C₂₄H₂₅N₈O₂+([M+H+]) 457.2095, found: 457.2098. Yellow solid, 48 mg, 51% for 2 steps.

### Example 47: 4-Amino-N-(4-methoxyphenyl)-2-(4-(4-methoxyphenyl)piperazin-1-yl)pyrimidine-5-carboxamide (33)

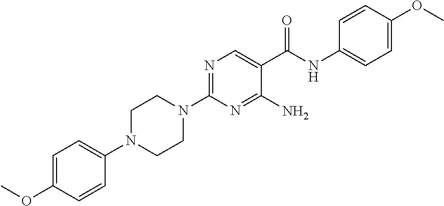

This compound was prepared using the procedure of Example 14 (R=4-methoxyphenyl). 1H NMR (400 MHz, DMSO-d6) δ 9.74 (s, 1H), 8.61 (s, 1H), 7.55 (s, 3H), 6.96-6.88 (m, 5H), 3.90 (s, 4H), 3.81 (s, 3H), 3.73 (s, 3H), 2.98 (s, 4H); 13C NMR (101 MHz, DMSO-d6) δ 165.4, 162.9, 160.8, 157.5, 155.4, 152.1, 141.1, 132.1, 122.8, 122.2, 120.8, 118.4, 113.7, 111.9, 98.9, 55.4, 55.2, 50.2, 43.4. LCMS: 457 [M+H]+; HRMS (ESI) calculated for C₂₃H₂₇N₆O₃+([M+H+]) 435.2139, found: 435.2151. White solid, 36 mg, 41% for 2 steps.

### Example 48: Method for the synthesis of 4-Amino-N-(4-fluorophenyl)-2-(4-(pyridin-2-yl)piperazin-1-yl)pyrimidine-5-carboxamide (34) and 4-Amino-2-(4-(pyridin-2-yl)piperazin-1-yl)-N-(4-(trifluoromethyl)phenyl)pyrimidine-5-carboxamide (35)

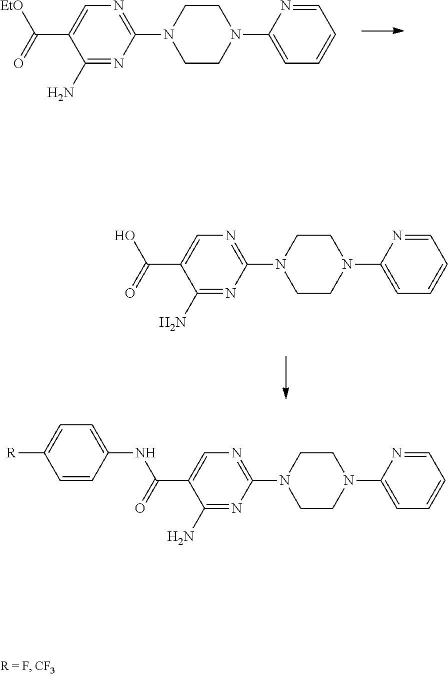

Step a: Compound 9 (0.20 mmol) was dissolved in EtOH and H₂O (2 mL/2 mL). The mixture was heated to 60° C. for 4 h. After cooling to room temperature, EtOH was removed under reduced pressure. 1 N HCl was used to make the solution to PH=7. The precipitated solid was filtered out and dried on a high vacuum for 12 h. The crude product was used for the next step directly.

Step b: A solution of the crude product above, DIPEA (77 mg, 0.6 mmol) and HBTU (0.21 mmol) in DMF (4 mL) was stirred for 15 min. at room temperature. Corresponding amine (0.21 mmol) in DMF (1 mL) was added to the solution. The mixture was heated to 50° C. and stirred overnight. After cooling to room temperature, the mixture was washed with 1 N Na₂CO₃ solution and extracted with DCM (3×15 mL), and dried over Na₂SO₄. Removed the solvent and the residue was purified by flash column chromatography on silica gel, eluting with 5% MeOH in DCM to afford the desired product as a solid.

R=F: 4-Amino-N-(4-fluorophenyl)-2-(4-(pyridin-2-yl)piperazin-1-yl)pyrimidine-5-carboxamide (34). 1H NMR (400 MHz, DMSO-d6) δ 9.90 (s, 1H), 8.63 (s, 1H), 8.13 (ddd, J=4.9, 2.1, 0.8 Hz, 1H), 7.70-7.62 (m, 2H), 7.56 (ddd, J=8.9, 7.1, 2.0 Hz, 2H), 7.19-7.11 (m, 2H), 6.87 (d, J=8.6 Hz, 1H), 6.66 (ddd, J=7.1, 4.8, 0.8 Hz, 1H), 3.94-3.82 (m, 4H), 3.60-3.51 (m, 4H); ¹³C NMR (101 MHz, DMSO-d6) δ 165.6, 162.9, 160.9, 158.9, 158.1 (d, J=241.9 Hz), 157.7, 147.6, 137.6, 135.4, 122.4 (d, J=7.8 Hz), 115.1 (d, J=22.1 Hz), 113.2, 107.2, 98.8, 44.4, 42.9. LCMS: 394 [M+H]+; HRMS (ESI) calculated for C₂₀H₂₁FN₇O+([M+H+]) 394.1786, found: 394.1782. White solid, 28 mg, 36%.

R=CF₃: 4-Amino-2-(4-(pyridin-2-yl)piperazin-1-yl)-N-(4-(trifluoromethyl)phenyl)pyrimidine-5-carboxamide (35). 1H NMR (400 MHz, DMSO-d6) δ 10.16 (s, 1H), 8.68 (s, 1H), 8.18-8.12 (m, 2H), 7.90 (d, J=8.5 Hz, 2H), 7.69 (d, J=8.6 Hz, 2H), 7.61-7.55 (m, 3H), 6.90-6.86 (m, 2H), 6.70-6.66 (m, 2H), 3.90 (t, J=5.4 Hz, 4H), 3.58 (t, J=5.4 Hz, 4H); ¹³C NMR (101 MHz, DMSO-d6) δ 166.5, 163.4, 159.4, 158.6, 148.1, 138.1, 126.3, 126.3, 120.5, 113.7, 107.7, 99.1, 44.9, 43.4. LCMS: 444 [M+H]+; HRMS (ESI) calculated for C₂₁H₂₁F₃N₇O₂+([M+H+]) 444.1754, found: 444.1755. Light-yellow solid, 5 mg, 5%.

### Example 49: Method for the synthesis of 4-Amino-2-(4-(pyridin-2-yl)piperazin-1-yl)-N-(4-(trifluoromethoxy)phenyl)pyrimidine-5-carboxamide (36)

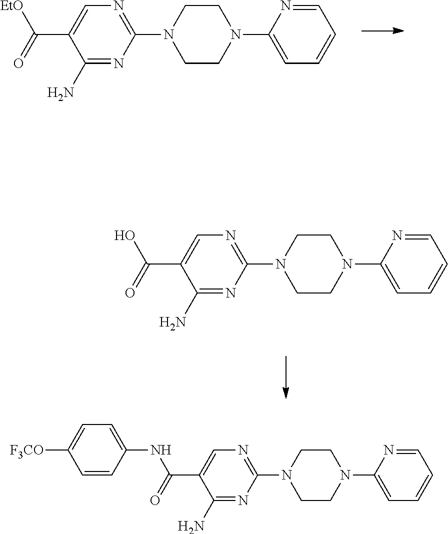 [SMILES: CCOC(=O)c1cnc(N2CCN(c3ccccn3)CC2)nc1N ; Nc1nc(N2CCN(c3ccccn3)CC2)ncc1C(=O)O ; Nc1nc(N2CCN(c3ccccn3)CC2)ncc1C(=O)Nc1ccc(OC(F)(F)F)cc1]

Step a: Compound 9 (0.20 mmol) was dissolved in EtOH and H₂O (2 mL/2 mL). The mixture was heated to 60° C. for 4 h. After cooling to room temperature, EtOH was removed under reduced pressure. 1 N HCl was used to make the solution to pH=7. The precipitated solid was filtered out and dried on a high vacuum for 12 h. The crude product was used for the next step directly.

Step b: A solution of the crude product above, Et₃N (0.6 mmol) and DMAP (0.02 mmol), and EDCl·HCl (0.21 mmol) in DMF (4 mL) was stirred for 15 min. at room temperature. 4-(Trifluoromethoxy)aniline (0.21 mmol) in DMF (1 mL) was added to the solution. The mixture was heated to 50° C. and stirred overnight. After cooling to room temperature, the mixture was washed with 1 N Na₂CO₃ solution and extracted with DCM (3×15 mL), and dried over Na₂SO₄. Removed the solvent and the residue was purified by flash column chromatography on silica gel, eluting with 5% MeOH in DCM to afford the desired product as a solid.

1H NMR (400 MHz, DMSO-d6) δ 10.02 (s, 1H), 8.64 (s, 1H), 8.13 (dd, J=5.0, 2.0 Hz, 1H), 7.76 (d, J=9.1 Hz, 2H), 7.56 (ddd, J=8.9, 7.0, 2.0 Hz, 2H), 7.32 (d, J=8.6 Hz, 2H), 6.87 (d, J=8.6 Hz, 1H), 6.66 (dd, J=7.1, 4.8 Hz, 1H), 3.88 (dd, J=6.9, 3.8 Hz, 4H), 3.57 (dd, J=6.4, 3.9 Hz, 4H); ¹³C NMR (101 MHz, DMSO-d6) δ 166.2, 163.4, 161.4, 159.4, 158.4, 148.0, 144.1, 138.9, 138.1, 122.2, 121.8, 119.4, 113.7, 107.7, 99.2, 44.9, 43.4. LCMS: 460 [M+H]+; HRMS (ESI) calculated for C₂₁H₂₁F₃N₇O₃+([M+H+]) 460.1703, found: 460.1711.Light-yellow solid, 32 mg, 36% for 2 steps.

### Example 50: Methods for the synthesis of 2-Hydroxyethyl 4-amino-2-(4-(pyridin-2-yl)piperazin-1-yl)pyrimidine-5-carboxylate (37)

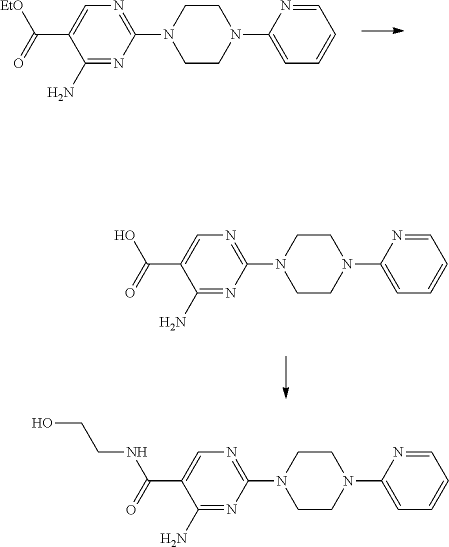 [SMILES: CCOC(=O)c1cnc(N2CCN(c3ccccn3)CC2)nc1N ; Nc1nc(N2CCN(c3ccccn3)CC2)ncc1C(=O)O ; Nc1nc(N2CCN(c3ccccn3)CC2)ncc1C(=O)NCCO]

Step a: Compound 9 (0.20 mmol) was dissolved in EtOH and H₂O (2 mL/2 mL). The mixture was heated to 60° C. for 4 h. After cooling to room temperature, EtOH was removed under reduced pressure. 1 N HCl was used to make the solution to pH=7. The precipitated solid was filtered out and dried on a high vacuum for 12 h. The crude product was used for the next step directly.

Step b: A solution of the crude product above, Et₃N (0.6 mmol) and DMAP (0.02 mmol), and EDCI·HCl (0.21 mmol) in DMF (4 mL) was stirred for 15 min. at room temperature. Ethane-1,2-diol (0.21 mmol) in DMF (1 mL) was added to the solution. The mixture was heated to 50° C. and stirred overnight. After cooling to room temperature, the mixture was washed with 1 N Na₂CO₃ solution and extracted with DCM (3×15 mL), and dried over Na2SO4. Removed the solvent and the residue was purified by flash column chromatography on silica gel, eluting with 5% MeOH in DCM to afford the desired product as a solid.

1H NMR (400 MHz, DMSO-d6) δ 8.61 (s, 1H), 8.13 (dd, J=5.0, 2.0 Hz, 1H), 7.55 (ddd, J=8.9, 7.0, 2.0 Hz, 1H), 7.44 (s, 2H), 6.86 (d, J=8.6 Hz, 1H), 6.66 (dd, J=7.1, 4.9 Hz, 1H), 4.89 (t, J=5.7 Hz, 1H), 4.18 (t, J=5.7 Hz, 2H), 3.90-3.87 (m, 4H), 3.66 (q, J=5.4 Hz, 2H), 3.59-3.50 (m, 4H); ¹³C NMR (101 MHz, DMSO-d6) δ 166.0, 162.8, 161.4, 160.8, 158.8, 147.6, 137.6, 113.2, 107.2, 94.9, 65.6, 59.1, 44.4, 42.9. LCMS: 345 [M+H]+; HRMS (ESI) calculated for C₁₆H₂₁N₆O₃+([M+H+]) 345.1670, found: 345.1677. White solid, 28 mg, 36% for 2 steps.

### Example 51: Methods for the synthesis of (4-Amino-2-(4-(pyridin-2-yl)piperazin-1-yl)pyrimidin-5-yl)methanol (38)

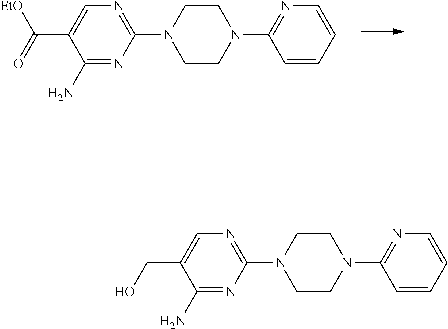 [SMILES: CCOC(=O)c1cnc(N2CCN(c3ccccn3)CC2)nc1N ; Nc1nc(N2CCN(c3ccccn3)CC2)ncc1CO]

A solution of ethyl 4-amino-2-(4-(pyridin-2-yl)piperazin-1-yl)pyrimidine-5-carboxylate (9) (65 mg, 0.20 mmol) in THE (4 mL) was cooled to 0° C. LiAlH₄ (0.4 mL, 1 M in THF) was added to the solution. The mixture was warmed to room temperature and stirred overnight. 10% NH₄Cl was added to quench the reaction. The mixture was extracted with DCM (10 mL×3). The combined solvents were dried with Na₂SO₄, and filtered through a celite pad. Removed the solvent and the crude product was purified by flash column chromatography on silica gel, eluting with 5% MeOH in DCM to afford the desired product as a white solid.

1H NMR (400 MHz, CDCl₃) δ 8.19 (dd, J=4.9, 1.9 Hz, 1H), 7.73 (s, 1H), 7.49 (ddd, J=8.9, 7.1, 2.0 Hz, 1H), 6.69-6.61 (m, 2H), 5.35 (s, 2H), 4.47 (s, 2H), 3.88-3.85 (m, 4H), 3.59-3.57 (m, 4H); ¹³C NMR (101 MHz, CDCl₃): δ 163.2, 159.4, 154.2, 148.0, 137.6, 113.5, 107.2, 106.1, 60.6, 45.1, 43.7. LCMS: 287 [M+H]+; HRMS (ESI) calculated for C₁₄H₁₉N₆O+([M+H+]) 287.1615, found: 287.1627. White solid, 54 mg, 62%.

### Example 52: Methods for the synthesis of 5-Methyl-2-(4-(pyridin-2-yl)piperazin-1-yl)pyrimidin-4-amine (39) and 2-(4-(Pyridin-2-yl)piperazin-1-yl)pyrimidin-4-amine (40)

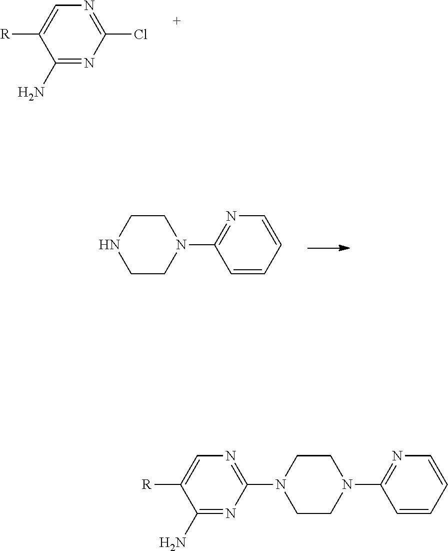

Step a: A solution of 2-chloropyrimidin-4-amine (0.20 mmol), 1-(pyridin-2-yl)piperazine (0.22 mmol) and Cs₂CO₃ (0.22 mmol) in DMF (3 mL) was heated to 110° C. overnight. Removed the solvent and the crude product was purified by flash column chromatography on silica gel, eluting with 5% MeOH in DCM to afford the desired product as a white solid.

5-Methyl-2-(4-(pyridin-2-yl)piperazin-1-yl)pyrimidin-4-amine (Compound 39). as described above (R=CH₃). 1H NMR (400 MHz, CDCl₃) δ 8.20 (ddd, J=4.9, 2.0, 0.9 Hz, 1H), 7.77 (s, 1H), 7.48 (ddd, J=8.8, 7.1, 2.0 Hz, 1H), 6.67 (d, J=8.6 Hz, 1H), 6.62 (ddd, J=7.1, 4.9, 0.8 Hz, 1H), 4.69 (s, 2H), 3.90-3.77 (m, 4H), 3.63-3.55 (m, 4H), 1.95 (s, 3H); ¹³C NMR (101 MHz, CDCl₃): δ 162.4, 160.9, 159.7, 155.3, 148.1, 137.6, 113.5, 107.3, 102.5, 45.2, 43.9, 13.0. LCMS: 271 [M+H]+; HRMS (ESI) calculated for C₁₄H₁₉N₆+([M+H+]271.1666, found: 271.1690. White solid, 60 mg, 73%.

2-(4-(Pyridin-2-yl)piperazin-1-yl)pyrimidin-4-amine (40). This compound was prepared as described above (R=H). 1H NMR (400 MHz, CDCl₃) δ 8.20 (d, J=4.9 Hz, 1H), 7.95 (d, J=5.6 Hz, 1H), 7.49 (t, J=7.9 Hz, 1H), 6.73-6.57 (m, 2H), 5.79 (d, J=5.6 Hz, 1H), 4.66 (s, 2H), 3.89 (s, 4H), 3.60 (s, 4H); ¹³C NMR (101 MHz, CDCl₃): δ 163.5, 161.8, 159.6, 156.7, 148.1, 137.7, 113.6, 107.3, 95.1, 45.2, 43.6. LCMS: 257 [M+H]+; HRMS (ESI) calculated for C₁₃H₁₇N₆+([M+H+]257.1509, found: 257.1523. White solid, 41 mg, 16%.

### Example 53: Methods for the synthesis of 4-Amino-2-(4-(pyridin-2-yl)piperazin-1-yl)pyrimidine-5-carbonitrile (41)

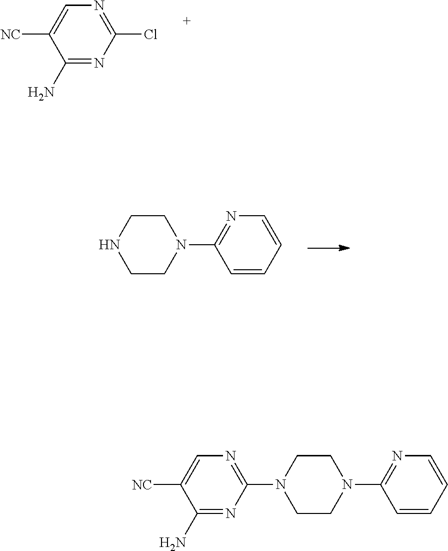 [SMILES: N#Cc1cnc(Cl)nc1N ; c1ccc(N2CCNCC2)nc1 ; N#Cc1cnc(N2CCN(c3ccccn3)CC2)nc1N]

4-Amino-2-chloropyrimidine-5-carbonitrile (46 mg, 0.30 mmol) and 1-(pyridin-2-yl)piperazine (0.45 mmol) was dissolved in 4 mL dry MeCN. Et₃N (91 mg, 0.90 mmol) was added to the solution. The mixture was stirred at room temperature overnight. Removed the solvent and the crude product was purified by flash column chromatography on silica gel, eluting with hexanes/ethyl acetate (with 5% 7N NH₃ in MeOH) (4:1 to 1:1) to afford the desired product as a white solid.

1H NMR (400 MHz, DMSO-d6) δ 8.29 (s, 1H), 8.12 (ddd, J=4.9, 2.1, 0.8 Hz, 1H), 7.55 (ddd, J=8.9, 7.0, 2.0 Hz, 1H), 7.29 (s, 2H), 6.86 (d, J=8.6 Hz, 1H), 6.73-6.58 (m, 1H), 3.85 (t, J=5.3 Hz, 4H), 3.59-3.50 (m, 4H); ¹³C NMR (101 MHz, DMSO-d6: δ 162.8, 161.9, 160.7, 158.8, 147.5, 137.6, 117.2, 113.2, 107.2, 78.5, 44.3, 42.8. LCMS: 282 [M+H]+; HRMS (ESI) calculated for C₁₄H₁₆N₇+([M+H+]) 282.1462, found: 282.1460. White solid, 76 mg, 90%.

### Example 54: Methods for the synthesis of N-(4-Methoxyphenyl)-2-(4-(pyridin-2-yl)piperazin-1-yl)pyrimidine-5-carboxamide (42)

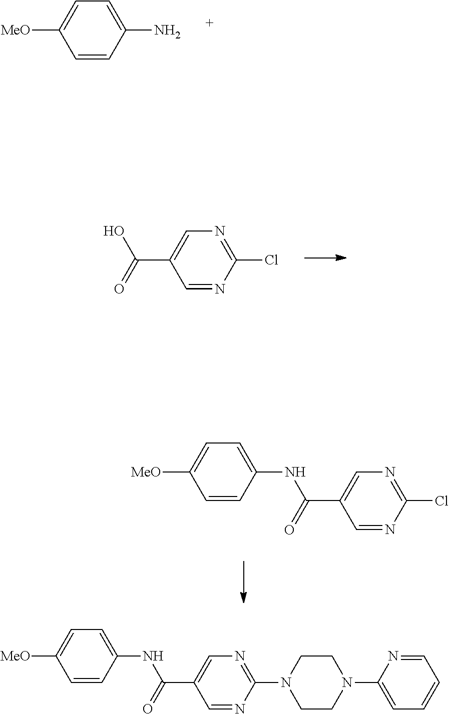 [SMILES: COc1ccc(N)cc1 ; O=C(O)c1cnc(Cl)nc1 ; COc1ccc(NC(=O)c2cnc(Cl)nc2)cc1 ; COc1ccc(NC(=O)c2cnc(N3CCN(c4ccccn4)CC3)nc2)cc1]

Step a: A solution of ethyl 2-chloropyrimidine-5-carboxylic acid (317 mg, 2.0 mmol) in DCM (6 mL) was cooled to O ° C. under N₂. SOCl₂ (357 mg, 3.0 mmol) was added to the solution. The mixture was stirred for 3 h at room temperature. The extra SOCl₂ was removed under vacuum. The residue was dissolved in 6 mL DCM. 4-methoxyaniline (246 mg, 2.0 mmol) was added to the mixture. The mixture was stirred at room temperature overnight. 10 mL saturated NaHCO₃ was added to quench the reaction. The mixture was extracted with DCM 3 times. Combine the organic phase and dried with Na₂SO₄. Removed the solvents and the crude product was purified by flash column chromatography on silica gel, eluting with 5% MeOH in DCM to afford the desired product as a white solid.

Step b: 2-chloro-N-(4-methoxyphenyl)pyrimidine-5-carboxamide (52 mg, 0.20 mmol) and 1-(pyridin-2-yl)piperazine (32 mg, 0.20 mmol) was dissolved in 4 mL dry MeCN. Et₃N (30 mg, 0.30 mmol) was added to the solution. The mixture was stirred at room temperature overnight. Removed the solvent and the crude product was purified by flash column chromatography on silica gel, eluting with hexanes/ethyl acetate (with 5% 7N NH₃ in MeOH) (4:1 to 1:1) to afford the desired product as a white solid.

1H NMR (400 MHz, DMSO-d6) δ 9.99 (s, 1H), 8.91 (s, 2H), 8.14 (dd, J=4.9, 2.0 Hz, 1H), 7.62 (d, J=9.0 Hz, 2H), 7.59-7.54 (m, 1H), 6.92 (d, J=9.0 Hz, 2H), 6.88 (d, J=8.7 Hz, 1H), 6.67 (dd, J=7.1, 4.9 Hz, 1H), 3.96 (t, J=5.3 Hz, 4H), 3.74 (s, 3H), 3.62 (t, J=5.3 Hz, 4H); ¹³C NMR (101 MHz, DMSO-d6) δ 162.2, 161.4, 158.8, 158.0, 155.5, 147.6, 137.7, 132.0, 121.9, 116.7, 113.8, 113.3, 107.3, 55.2, 44.3, 43.2. LCMS: 391 [M+H]+; HRMS (ESI) calculated for C₂₁H₂₃N₆O₂+([M+H]+) 391.1877, found: 391.1879. White solid, 59 mg, 76%.

### Example 55: 2-Chloro-N-(4-methoxyphenyl)pyrimidine-5-carboxamide (43)

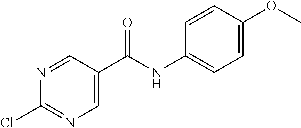 [SMILES: COc1ccc(NC(=O)c2cnc(Cl)nc2)cc1]

This compound was prepared as described herein. 1H NMR (400 MHz, DMSO-d6) δ 10.50 (s, 1H), 9.21 (s, 2H), 7.65 (d, J=9.0 Hz, 2H), 6.96 (d, J=9.0 Hz, 2H), 3.75 (s, 3H); ¹³C NMR (101 MHz, DMSO-d6) δ 162.2, 161.1, 160.1, 156.5, 131.8, 128.1, 122.4, 114.4, 55.7. LCMS: 264 [M+H]+. White solid, 101 mg, 19%.

### Example 56: 4-methoxy-N-(4-(6-(pyridin-2-yl)-2,6-diazaspiro[3.3]heptan-2-yl)phenyl)benzamide (117)

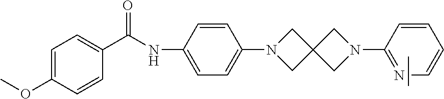

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(6-(pyridin-2-yl)-2,6-diazaspiro[3.3]heptan-2-yl)phenyl)benzamide (30 mg, 52% yield) as a white solid. (1H NMR, 400 MHz, DMSO-d6): δ=9.83 (s, 1H), 8.07 (dd, J1=5.0 Hz, J2=1.0 Hz, 1H), 7.93 (d, J=8.8 Hz, 2H), 7.55 (d, J=8.8 Hz, 2H), 7.03 (ddd, J1=8.2 Hz, J2=7.1 Hz, J3=1.9 Hz, 1H), 7.03 (d, J=8.8 Hz, 2H), 6.64 (dd, J1=6.8 Hz, J2=5.2 Hz, 1H), 6.45 (d, J=8.8 Hz, 2H), 6.39 (d, J=8.3 Hz, 1H), 4.10 (s, 4H), 3.96 (s, 4H), 3.83 (s, 3H); ¹³C NMR, 100 MHz, DMSO-d6: δ=164.2, 161.6, 160.4, 148.1, 147.7, 137.0, 129.9, 129.3, 127.2, 121.8, 113.5, 112.9, 111.5, 106.0, 62.0, 60.5, 55.3, 34.1; ESI-MS m/z calculated for C₂₄H₂₆N₄O₂+[M+2H]+402.5; found 402.7 HRMS (ESI) for C₂₄H₂₅N₄O₂+[M+H]+ 401.1978; found 401.1979

### Example 57: 4-methoxy-N-(4-(7-(pyridin-2-yl)-2,7-diazaspiro[3.5]nonan-2-yl)phenyl)benzamide (118)

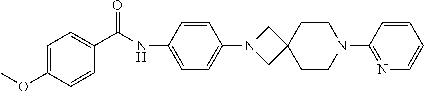 [SMILES: COc1ccc(C(=O)Nc2ccc(N3CC4(CCN(c5ccccn5)CC4)C3)cc2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(7-(pyridin-2-yl)-2,7-diazaspiro[3.5]nonan-2-yl)phenyl)benzamide (103 mg, 75% yield) as a white solid. (1H NMR, 400 MHz, DMSO-d6): δ=9.81 (s, 1H), 8.10 (dd, J1=4.8 Hz, J2=1.4 Hz, 1H), 7.93 (dt, J1=8.8 Hz, J2=2.8 Hz, 2H), 7.53 (d, J=8.8 Hz, 2H), 7.50 (ddd, J1=8.7 Hz, J2=6.9 Hz, J3=2.0 Hz, 1H), 7.03 (dt, J1=8.9 Hz, J2=2.9 Hz, 2H), 6.84 (d, J=8.6 Hz, 1H), 6.59 (dd, J1=6.8 Hz, J2=5.3 Hz, 1H), 6.42 (d, J=8.9 Hz, 2H), 3.83 (s, 3H) 3.59 (s, 4H), 3.53 (t, J=5.3 Hz, 4H), 1.78 (t, J=5.4 Hz, 4H); ¹³C NMR, 100 MHz, DMSO-d6: δ=164.2, 161.6, 158.8, 148.4, 147.6, 137.4, 129.4, 129.3, 127.2, 121.9, 113.5, 112.4, 111.0, 107.1, 61.6, 55.3, 42.0, 34.7, 34.5; ESI-MS m/z calculated for C₂₆H₃₀N₄O₂+[M+2H]+430.6; found 430.6

### Example 58: 4-methoxy-N-(4-(7-(3-nitrophenyl)-2,7-diazaspiro[3.5]nonan-2-yl)phenyl)benzamide (119)

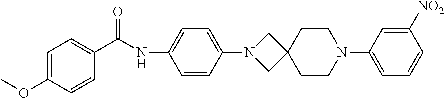 [SMILES: COc1ccc(C(=O)Nc2ccc(N3CC4(CCN(c5cccc([N+](=O)[O-])c5)CC4)C3)cc2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(7-(3-nitrophenyl)-2,7-diazaspiro[3.5]nonan-2-yl)phenyl)benzamide (97 mg, 59% yield) as a yellow solid. (1H NMR, 400 MHz, DMSO-d6): δ=9.82 (s, 1H), 7.94 (d, J=8.8 Hz, 2H), 7.68 (t, J=2.2 Hz, 1H), 7.57-7.53 (m, 3H), 7.49-7.43 (m, 2H), 7.04 (d, J=8.9 Hz, 2H), 6.43 (d, J=8.8 Hz, 2H), 3.83 (s, 3H), 3.61 (s, 4H), 3.33 (t, J=5.4 Hz, 4H), 1.88 (t, J=5.3 Hz, 4H); ¹³C NMR, 100 MHz, DMSO-d6: δ=164.2, 161.6, 151.5, 148.9, 148.4, 130.1, 129.4, 129.3, 127.2, 121.9, 121.7, 113.5, 112.3, 111.0, 108.6, 61.5, 55.3, 45.2, 34.5, 34.0; ESI-MS m/z calculated for C₂₇H₂₉N₄O₄+[M+H]+ 473.6; found 473.6

### Example 59: 4-methoxy-N-(4-(7-(pyridin-2-yl)-2,7-diazaspiro[4.4]nonan-2-yl)phenyl)benzamide (120)

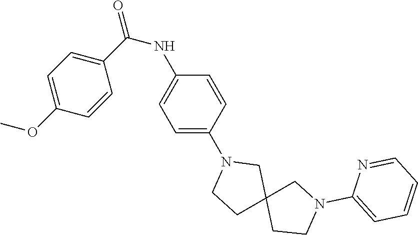 [SMILES: COc1ccc(C(=O)Nc2ccc(N3CCC4(CCN(c5ccccn5)C4)C3)cc2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(7-(pyridin-2-yl)-2,7-diazaspiro[4.4]nonan-2-yl)phenyl)benzamide (18 mg, 79% yield) as a white solid. (1H NMR, 400 MHz, DMSO-d6): δ=9.79 (s, 1H), 8.05 (dd, J1=4.9 Hz, J2=1.3 Hz, 1H), 7.94 (dt, J1=8.8 Hz, J2=2.8 Hz, 2H), 7.54 (d, J=8.9 Hz, 2H), 7.47 (ddd, J1=8.6 Hz, J2=6.9 Hz, J3=2.0 Hz, 1H), 7. 03 (dt, J1=8.8 Hz, J2=2.9 Hz, 2H), 6.55-6.51 (m, 3H), 6.44 (d, J=8.5 Hz, 1H), 3.83 (s, 3H), 3.51 (t, J=7.0 Hz, 2H), 3.46-3.35 (m, 4H), 3.26 (dd, J1=12.2 Hz, J2=9.4 Hz, 2H), 2.07-1.97 (m, 4H); ¹³C NMR, 100 MHz, DMSO-d6: δ=164.0, 161.5, 156.9, 147.8, 144.4, 136.9, 129.3, 128.1, 127.3, 122.1, 113.4, 111.2, 106.2, 57.2, 56.0, 55.3, 54.9, 48.0, 47.0, 45.7, 34.8, 34.7; ESI-MS m/z calculated for C₂₆H₃₀N₄O₂+[M+2H]+430.6; found 430.5 HRMS (ESI) for C₂₆H₂₉N₄O₂+[M+H]+ 429.2291; found 429.2285

### Example 60: 4-methoxy-N-(4-(5-(pyridin-2-yl)hexahydropyrrolo[3,4-b]pyrrol-1(2H)-yl)phenyl)benzamide (100)

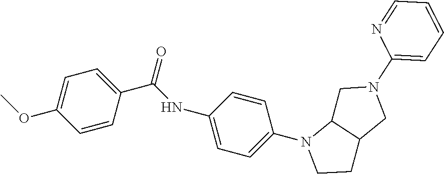 [SMILES: COc1ccc(C(=O)Nc2ccc(N3CCC4CN(c5ccccn5)CC43)cc2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(5-(pyridin-2-yl)hexahydropyrrolo[3,4-b]pyrrol-1(2H)-yl)phenyl)benzamide (9 mg, 26% yield) as a yellow solid. (1H NMR, 400 MHz, DMSO-d6): δ=9.83 (s, 1H), 8.05 (dd, J1=4.9 Hz, J2=1.3 Hz, 1H), 7.95 (dt, J1=8.8 Hz, J2=2.8 Hz, 2H), 7.57 (d, J=8.9 Hz, 2H), 7.47 (ddd, J1=8.6 Hz, J2=7.0 Hz, J3=2.0 Hz, 1H), 7.04 (dt, J1=8.9 Hz, J2=2.8 Hz, 2H), 6.59-6.55 (m, 3H), 6.48 (d, J=8.5 Hz, 1H), 4.29 (td, J1=6.7 Hz, J2=2.6 Hz, 1H), 3.83 (s, 3H), 3.70 (dd, J1=11.2 Hz, J2=6.2 Hz, 1H), 3.58 (dd, J1=10.6 Hz, J2=8.0 Hz, 1H), 3.51 (dt, J1=9.0 Hz, J2=6.9 Hz, 1H), 3.44 (dd, J1=11.2 Hz, J2=2.6 Hz, 1H), 3.38 (dd, J1=10.6 Hz, J2=4.6 Hz, 1H), 3.31-3.28 (m, 1H), 3.20-3.11 (m, 1H), 2.23-2.14 (m, 1H), 2.00-1.87 (m, 1H); ¹³C NMR, 100 MHz, DMSO-d6: δ=164.1, 161.5, 157.4, 147.8, 143.4, 137.0, 129.3, 128.6, 127.3, 122.1, 113.4, 112.0, 111.8, 106.9, 62.0, 55.3, 54.9, 52.3, 50.8, 48.0, 41.7, 29.5; ESI-MS m/z calculated for C₂₅H₂₈N₄O₂+[M+2H]+416.5; found 416.4 HRMS (ESI) for C₂₅H₂₇N₄O₂+[M+H]+ 415.2134; found 415.2141

### Example 61: 4-methoxy-N-(4-(2-(pyridin-2-yl)-2,8-diazaspiro[4.5]decan-8-yl)phenyl)benzamide (101)

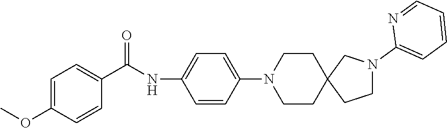 [SMILES: COc1ccc(C(=O)Nc2ccc(N3CCC4(CC3)CCN(c3ccccn3)C4)cc2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(2-(pyridin-2-yl)-2,8-diazaspiro[4.5]decan-8-yl)phenyl)benzamide (12 mg, 25% yield) as a white solid. (1H NMR, 400 MHz, DMSO-d6): δ=9.87 (s, 1H), 8.04 (dd, J1=5.0 Hz, J2=1.3 Hz, 1H), 7.94 (dt, J1=8.9 Hz, J2=2.8 Hz, 2H), 7.59 (d, J=9.1 Hz, 2H), 7.46 (ddd, J1=9.0 Hz, J2=7.0 Hz, J3=2.0 Hz, 1H), 7.04 (dt, J1=8.9 Hz, J2=2.8 Hz, 2H), 6.94 (d, J=9.1 Hz, 2H), 6.52 (dd, J1=6.6 Hz, J2=5.1 Hz, 1H), 6.43 (d, J=8.5 Hz, 1H), 3.83 (s, 3H), 3.45 (t, J=7.0 Hz, 2H), 3.31 (s, 2H), 3.24-3.19 (m, 2H), 3.13-3.07 (m, 2H), 1.88 (t, J=7.0 Hz, 2H), 1.73-1.63, 4H) (¹³C NMR, 100 MHz, DMSO-d6): δ=164.3, 161.6, 157.0, 147.8, 147.6, 136.8, 131.0, 129.4, 127.2, 121.5, 115.9, 113.5, 111.0, 106.2, 56.1, 55.4, 54.9, 46.6, 44.7, 35.2, 34.2, 30.7; ESI-MS m/z calculated for C₂₇H₃₁N₄O₂+[M+H]+ 443.6; found 443.5 HRMS (ESI) for C₂₇H₃₁N₄O₂+[M+H]+ 443.2447; found 443.2457

### Example 62: 4-methoxy-N-(4-(5-(pyridin-2-yl)-2,5-diazabicyclo[2.2.1]heptan-2-yl)phenyl)benzamide (102)

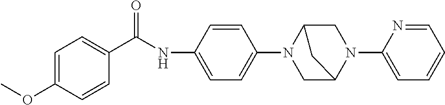 [SMILES: COc1ccc(C(=O)Nc2ccc(N3CC4CC3CN4c3ccccn3)cc2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(5-(pyridin-2-yl)-2,5-diazabicyclo[2.2.1]heptan-2-yl)phenyl)benzamide (25 mg, 45% yield) as a white solid. (1H NMR, 400 MHz, DMSO-d6): δ=9.78 (s, 1H), 8.02 (dd, J1=5.0 Hz, J2=1.2 Hz, 1H), 7.92 (dt, J1=8.8 Hz, J2=2.6 Hz, 2H), 7.51 (d, J=8.9 Hz, 2H), 7.43 (ddd, J1=8.6 Hz, J2=7.0 Hz, J3=1.9 Hz, 1H), 7.03 (dt, J1=8.8 Hz, J2=2.8 Hz, 2H), 6.57 (d, J=9.0 Hz, 2H), 6.52 (dd, J1=6.5 Hz, J2=5.1 Hz, 1H), 6.46 (d, J=8.5 Hz, 1H), 4.85 (s, 1H), 4.59 (s, 1H), 3.83 (s, 3H), 3.61 (dd, J1=8.8 Hz, J2=1.8 Hz, 1H), 3.51 (dd, J1=9.5 Hz, J2=1.5 Hz, 1H), 3.32 (d, J=11.5 Hz, 2H), 2.97 (d, J=8.9 Hz, 1H), 2.04 (dd, J1=14.7 Hz, J2=9.4 Hz, 2H); ¹³C NMR, 100 MHz, DMSO-d6: δ=164.1, 161.5, 156.8, 147.8, 143.5, 137.1, 129.3, 128.7, 127.3, 123.0, 113.5, 112.4, 111.8, 106.9, 56.8, 56.1, 55.9, 55.4, 52.4, 37.2; ESI-MS m/z calculated for C₂₄H₂₆N₄O₂+[M+2H]+402.5; found 402.4 HRMS (ESI) for C₂₄H₂₅N₄O₂+[M+H]+ 401.1978; found 401.2000

### Example 63: 4-methoxy-N-(4-(3-(pyridin-2-yl)-3,8-diazabicyclo[3.2.1]octan-8-yl)phenyl)benzamide (103)

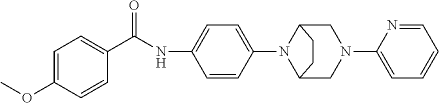 [SMILES: COc1ccc(C(=O)Nc2ccc(N3C4CCC3CN(c3ccccn3)C4)cc2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(3-(pyridin-2-yl)-3,8-diazabicyclo[3.2.1]octan-8-yl)phenyl)benzamide (960 mg, 63% yield) as a white solid. (1H NMR, 400 MHz, DMSO-d6): δ=9.83 (s, 1H), 8.07 (dd, J1=4.9 Hz, J2=1.3 Hz, 1H), 7.92 (dt, J1=8.8 Hz, J2=2.8 Hz, 2H), 7.58 (d, J=9.0 Hz, 2H), 7.49 (ddd, J1=8.7 Hz, J2=7.0 Hz, J3=2.0 Hz, 1H), 7.03 (dt, J1=8.9 Hz, J2=2.9 Hz, 2H), 6.92 (d, J=9.1 Hz, 2H), 6.68 (d, J=8.6 Hz, 1H), 6.61 (dd, J1=6.7 Hz, J2=5.0 Hz, 1H), 4.40 (s, 2H), 3.83 (s, 3H), 3.61 (d, J=10.7 Hz, 2H), 3.11 (d, J=10.7 Hz, 2H), 1.95 (t, J=4.0 Hz, 2H), 1.77 (q, J=5.9 Hz, 2H); ¹³C NMR, 100 MHz, DMSO-d6: δ=164.2, 161.6, 159.9, 147.4, 143.0, 137.2, 129.8, 129.3, 127.2, 122.0, 115.5, 113.5, 112.7, 106.3, 55.4, 53.9, 46.8, 27.2; ESI-MS m/z calculated for C₂₅H₂₈N₄O₂+[M+2H]+416.5; found 416.5 HRMS (ESI) for C₂₅H₂₇N₄O₂+[M+H]+ 415.2134; found 415.2148

### Example 64: 4-methoxy-N-(4-(3-(quinolin-3-yl)-3,8-diazabicyclo[3.2.1]octan-8-yl)phenyl)benzamide (104)

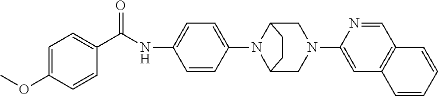 [SMILES: COc1ccc(C(=O)Nc2ccc(N3C4CCC3CN(c3cc5ccccc5cn3)C4)cc2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(3-(quinolin-3-yl)-3,8-diazabicyclo[3.2.1]octan-8-yl)phenyl)benzamide (69 mg, 74% yield) as a yellow solid. (1H NMR, 400 MHz, DMSO-d6): δ=9.84 (s, 1H), 8.83 (d, J=2.9 Hz, 1H), 7.92 (dt, J1=8.9 Hz, J2=2.8 Hz, 2H), 7.85-7.81 (m, 1H), 7.74-7.71 (m, 1H), 7.59 (d, J=9.0 Hz, 2H), 7.44-7.42 (m, 2H), 7.39 (d, J=2.8 Hz, 1H), 7.03 (dt, J1=8.9 Hz, J2=2.9 Hz, 2H), 6.95 (d, J=9.1 Hz, 2H), 4.46 (s, 2H), 3.82 (s, 3H), 3.56 (d, J=10.0 Hz, 2H), 3.16 (d, J=10.4 Hz, 2H), 2.02-1.94 (m, 4H); ¹³C NMR, 100 MHz, DMSO-d6: δ=164.2, 161.6, 144.7, 142.9, 142.5, 141.6, 129.9, 129.3, 128.6, 128.3, 127.2, 126.6, 126.5, 125.3, 122.0, 115.6, 113.7, 113.5, 55.4, 54.9, 54.1, 49.1, 27.3; ESI-MS m/z calculated for C₂₉H₃₀N₄O₂+[M+2H]+466.6; found 466.4 HRMS (ESI) for C₂₉H₂₉N₄O₂+[M+H]+ 465.2291; found 465.2300

### Example 65: 4-methoxy-N-(4-(3-(quinoxalin-2-yl)-3,8-diazabicyclo[3.2.1]octan-8-yl)phenyl)benzamide (105)

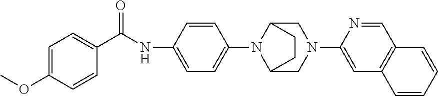 [SMILES: COc1ccc(C(=O)Nc2ccc(N3C4CCC3CN(c3cc5ccccc5cn3)C4)cc2)cc1]

This compound was prepared as described herein. Purification by flash chromatography using CH₂Cl₂/7N NH₃ in MeOH=40:1 gave 4-methoxy-N-(4-(3-(quinoxalin-2-yl)-3,8-diazabicyclo[3.2.1]octan-8-yl)phenyl)benzamide (36 mg, 52%) as a yellow solid. (1H NMR, 400 MHz, DMSO-d6): δ=9.85 (s, 1H), 8.73 (s, 1H), 7.92 (d, J=2.8 Hz, 2H), 7.81 (d, J=8.0 Hz, 1H), 7.62-7.58 (m, 4H), 7.40-7.36 (m, 1H), 7.03 (dt, J1=8.9 Hz, J2=2.8 Hz, 2H), 6.97 (d, J=9.0 Hz, 2H), 4.47 (s, 2H), 4.12 (d, J=11.7 Hz, 2H), 3.82 (s, 3H), 3.36 (d, J=11.5 Hz, 2H), 1.98 (t, J=4.2 Hz, 2H), 1.82 (q, J=5.9 Hz, 2H); ¹³C NMR, 100 MHz, DMSO-d6: δ=164.2, 161.6, 153.1, 142.8, 141.0, 136.4, 136.3, 130.0, 129.9, 129.3, 128.3, 127.2, 125.9, 124.2, 122.1, 122.0, 115.6, 113.4, 55.3, 54.9, 53.9, 46.3, 26.9; ESI-MS m/z calculated for C₂₈H₂₉N₅O₂+[M+2H]+467.6; found 467.5 HRMS (ESI) for C₂₈H₂₈N₅O₂+[M+H]+ 466.2243; found 466.2240

### Example 66: 4-methoxy-N-(4-(3-(pyridin-2-yl)-3,9-diazabicyclo[3.3.1]nonan-9-yl)phenyl)benzamide (106)

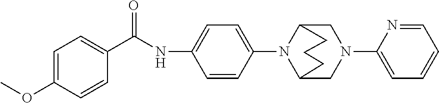 [SMILES: COc1ccc(C(=O)Nc2ccc(N3C4CCCC3CN(c3ccccn3)C4)cc2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(3-(pyridin-2-yl)-3,9-diazabicyclo[3.3.1]nonan-9-yl)phenyl)benzamide (7 mg, 19% yield) as a white solid. (1H NMR, 400 MHz, DMSO-d6): δ=9.88 (s, 1H), 8.13 (dd, J1=4.9 Hz, J2=1.4 Hz, 1H), 7.95 (dt, J1=8.9 Hz, J2=2.8 Hz, 2H), 7.61 (d, J=9.0 Hz, 2H), 7.54 (ddd, J1=8.7 Hz, J2=6.9 Hz, J3=2.0 Hz, 1H), 7.05 (dt, J1=8.9 Hz, J2=2.8 Hz, 2H), 6.93 (d, J=9.1 Hz, 2H), 6.87 (d, J=8.7 Hz, 1H), 6.58 (dd, J1=6.9 Hz, J2=5.0 Hz, 1H), 4.64 (s, 2H), 3.84 (s, 3H), 3.77 (d, J=11.0 Hz, 2H), 2.97 (dd, J1=11.4 Hz, J2=3.2 Hz, 2H), 2.48-2.42 (m, 1H), 1.80 (d, J=6.2 Hz, 4H), 1.50-1.47 (m, 1H); ¹³C NMR, 100 MHz, DMSO-d6: δ=164.2, 161.6, 157.4, 147.9, 146.8, 137.6, 130.5, 129.4, 127.2, 121.6, 114.4, 113.5, 112.1, 107.0, 55.4, 51.9, 46.5, 27.8, 19.8; ESI-MS m/z calculated for C₂₆H₃₀N₄O₂+[M+2H]+430.6; found 430.5 HRMS (ESI) for C₂₆H₂₉N₄O₂+[M+H]+ 429.2291; found 429.2291

### Example 67: 4-methoxy-N-(4-(4-(pyridin-2-yl)-1,4-diazepan-1-yl)phenyl)benzamide (107)

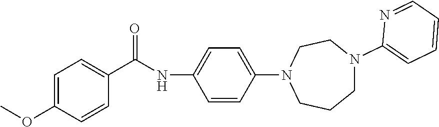 [SMILES: COc1ccc(C(=O)Nc2ccc(N3CCCN(c4ccccn4)CC3)cc2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(4-(pyridin-2-yl)-1,4-diazepan-1-yl)phenyl)benzamide (24 mg, 40% yield) as a white solid. (1H NMR, 400 MHz, DMSO-d6): δ=9.77 (s, 1H), 8.05 (dd, J1=4.9 Hz, J2=1.4 Hz, 1H), 7.92 (dt, J1=8.8 Hz, J2=2.8 Hz, 2H), 7.52 (d, J=9.0 Hz, 2H), 7.46 (ddd, J1=8.7 Hz, J2=6.9 Hz, J3=2.0 Hz, 1H), 7.03 (dt, J1=8.9 Hz, J2=2.9 Hz, 2H), 6.73 (d, J=9.1 Hz, 2H), 6.66 (d, J=8.6 Hz, 1H), 6.51 (dd, J1=6.9 Hz, J2=5.1 Hz, 1H), 3.83 (s, 3H), 3.81 (t, J=5.7 Hz, 2H), 3.59 (t, J=5.2 Hz, 2H), 3.48 (t, J=6.0 Hz, 2H), 3.40 (t, J=5.9 Hz, 2H), 2.00-1.94 (m, 2H); ¹³C NMR, 100 MHz, DMSO-d6: δ=164.1, 161.5, 157.1, 147.8, 143.8, 137.4, 129.3, 128.2, 127.3, 122.3, 113.5, 111.34, 111.29, 105.6, 55.4, 48.8, 47.2, 46.1, 45.6, 23.8; ESI-MS m/z calculated for C₂₄H₂₈N₄O₂+[M+2H]+404.5; found 404.5 HRMS (ESI) for C₂₄H₂₇N₄O₂+[M+H]+ 403.2134; found 403.2117

### Example 68: N-(4-(4-(5-chloropyridin-2-yl)piperazin-1-yl)phenyl)-4-methoxybenzamide (108)

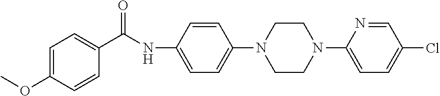 [SMILES: COc1ccc(C(=O)Nc2ccc(N3CCN(c4ccc(Cl)cn4)CC3)cc2)cc1]

This compound was prepared as described herein. Purification by flash chromatography using 0% to 50% EtOAc/n-Hexane with gradient elution and CH₂Cl₂/7N NH₃ in MeOH=40:1 gave N-(4-(4-(5-chloropyridin-2-yl)piperazin-1-yl)phenyl)-4-methoxybenzamide (20 mg, 34% yield) as a white solid. (1H NMR, 400 MHz, DMSO-d6): δ=9.91 (s, 1H), 8.14 (d, J=2.7 Hz, 1H), 7.94 (dt, J1=8.9 Hz, J2=2.8 Hz, 2H), 7.62 (dd, J1=9.1 Hz, J2=2.4 Hz, 3H), 7.04 (dt, J1=8.9 Hz, J2=2.8 Hz, 2H), 6.98 (d, J=9.1 Hz, 2H), 6.95 (d, J=9.2 Hz, 1H), 3.83 (s, 3H), 3.63 (t, J=4.8 Hz, 2H), 3.19 (t, J=5.1 Hz, 2H); ¹³C NMR, 100 MHz, DMSO-d6: δ=164.3, 161.7, 157.5, 147.2, 145.6, 137.2, 131.6, 129.4, 127.1, 121.4, 119.0, 116.0, 113.5, 108.6, 55.4, 48.5, 44.7; ESI-MS m/z calculated for C₂₃H₂₄C₁N₄O₂+[M+H]+ 423.9; found 423.4 HRMS (ESI) for C₂₃H₂₄C₁N₄₀₂+[M+H]+ 423.1588; found 423.1598

### Example 69: 4-methoxy-N-(5-(4-(pyridin-2-yl)piperazin-1-yl)pyridin-2-yl)benzamide (109)

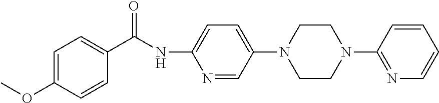 [SMILES: COc1ccc(C(=O)Nc2ccc(N3CCN(c4ccccn4)CC3)cn2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(5-(4-(pyridin-2-yl)piperazin-1-yl)pyridin-2-yl)benzamide (42 mg, 63% yield) as a white solid. (1H NMR, 400 MHz, DMSO-d6): δ=10.42 (s, 1H), 8.15-8.12 (m, 2H), 8.05-8.02 (m, 3H), 7.56 (ddd, J1=8.7 Hz, J2=7.0 Hz, J3=2.0 Hz, 1H), 7.51 (dd, J1=9.1 Hz, J2=3.0 Hz, 1H), 7.02 (d, J=8.8 Hz, 2H), 6.91 (d, J=8.6 Hz, 1H), 6.67 (dd, J1=7.0 Hz, J2=5.0 Hz, 1H), 3.83 (s, 3H), 3.65 (t, J=4.8 Hz, 2H), 3.26 (t, J=5.1 Hz, 2H); ¹³C NMR (100 MHz, DMSO-d6): δ=174.2, 171.6, 168.6, 157.2, 154.4, 153.4, 147.2, 145.0, 139.4, 136.0, 134.8, 124.8, 123.2, 122.9, 117.0, 65.0, 57.8, 54.1; ESI-MS m/z calculated for C₂₂H₂₄N₅O₂+[M+H]+ 390.5; found 390.4 HRMS (ESI) for C₂₂H₂₄N₅O₂+[M+H]+ 390.1930; found 390.1929

### Example 70: 4-methoxy-N-(5-(3-(pyridin-2-yl)-3,8-diazabicyclo[3.2.1]octan-8-yl)pyridin-2-yl)benzamide (110)

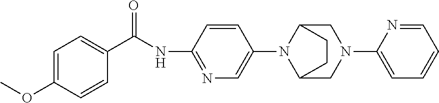 [SMILES: COc1ccc(C(=O)Nc2ccc(N3C4CCC3CN(c3ccccn3)C4)cn2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(5-(3-(pyridin-2-yl)-3,8-diazabicyclo[3.2.1]octan-8-yl)pyridin-2-yl)benzamide (17 mg, 23% yield) as a yellow oil. (1H NMR, 400 MHz, MeOD): δ=8.05 (dd, J1=4.9 Hz, J2=1.3 Hz, 2H), 7.98 (d, J=8.9 Hz, 1H), 7.93 (d, J=8.8 Hz, 2H), 7.53 (ddd, J1=8.7 Hz, J2=7.0 Hz, J3=1.9 Hz, 1H), 7.46 (dd, J1=9.1 Hz, J2=2.4 Hz, 1H), 7.03 (d, J=8.8 Hz, 2H), 6.72 (d, J=8.7 Hz, 1H), 6.66 (dd, J1=6.9 Hz, J2=5.2 Hz, 1H), 4.44 (brs, 2H), 3.86 (s, 3H), 3.80 (dd, J1=12.2 Hz, J2=1.4 Hz, 2H), 3.21 (d, J=11.6 Hz, 2H), 2.06 (t, J=4.0 Hz, 2H), 1.91 (d, J=5.8 Hz, 2H) (¹³C NMR, 100 MHz, DMSO-d6): δ=167.8, 164.5, 161.7, 158.3, 148.2, 144.3, 139.3, 136.7, 130.7, 126.8, 114.5, 109.4, 108.6, 56.3, 56.1, 28.5; ESI-MS m/z calculated for C₂₄H₂₆N₅O₂+[M+H]+ 416.5; found 416.4 HRMS (ESI) for C₂₄H₂₆N₅O₂+[M+H]+ 416.2087; found 416.2087.

### Example 71: 4-methoxy-N-(5-(4-(pyridin-2-yl)-1,4-diazepan-1-yl)pyridin-2-yl)benzamide (111)

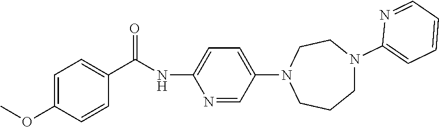 [SMILES: COc1ccc(C(=O)Nc2ccc(N3CCCN(c4ccccn4)CC3)cn2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(5-(4-(pyridin-2-yl)-1,4-diazepan-1-yl)pyridin-2-yl)benzamide (60 mg, 68% yield) as a white solid. 1H NMR (400 MHz, MeOD): δ=8.00 (d, J=4.4 Hz, 1H), 7.92 (d, J=8.7 Hz, 2H), 7.86 (d, J=9.0 Hz, 2H), 7.47 (ddd, J1=8.9 Hz, J2=6.7 Hz, J3=1.4 Hz, 1H), 7.26 (dd, J1=9.1 Hz, J2=2.9 Hz, 1H), 7.02 (d, J=8.7 Hz, 2H), 6.68 (d, J=8.7 Hz, 1H), 6.54 (t, J=5.9 Hz, 1H), 3.88 (t, J=4.9 Hz, 5H), 3.69 (t, J=5.5 Hz, 2H), 3.57 (t, J=6.0 Hz, 2H), 3.51 (t, J=6.0 Hz, 2H), 2.09-2.03 (m, 2H); ¹³C NMR, 100 MHz, MeOD: 6=167.8, 164.4, 158.8, 148.5, 143.0, 142.5, 139.4, 132.3, 130.6, 122.4, 118.3, 115.0, 113.1, 107.8, 56.1, 54.9, 47.9, 47.7, 25.0; ESI-MS m/z calculated for C₂₃H₂₆N₅O₂+[M+H]+ 404.5; found 404.4 HRMS (ESI) for C₂₃H₂₆N₅O₂+[M+H]+ 404.2087; found 404.2084

### Example 72: 4-methoxy-N-(6-(3-(pyridin-2-yl)-3,8-diazabicyclo[3.2.1]octan-8-yl)pyridin-3-yl)benzamide (112)

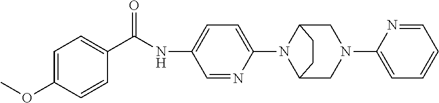 [SMILES: COc1ccc(C(=O)Nc2ccc(N3C4CCC3CN(c3ccccn3)C4)nc2)cc1]

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(6-(3-(pyridin-2-yl)-3,8-diazabicyclo[3.2.1]octan-8-yl)pyridin-3-yl)benzamide (83 mg, 13% yield) as a white solid. (1H NMR, 400 MHz, DMSO-d6): δ=9.95 (s, 1H), 8.45 (d, J=2.6 Hz, 1H), 8.08 (dd, J1=4.8 Hz, J2=1.4 Hz, 1H), 7.94 (dt, J1=8.8 Hz, J2=2.9 Hz, 2H), 7.90 (dd, J1=9.0 Hz, J2=2.6 Hz, 1H), 7.49 (ddd, J1=8.7 Hz, J2=7.0 Hz, J3=2.0 Hz, 1H), 7.05 (dt, J1=8.9 Hz, J2=2.9 Hz, 2H), 6.91 (d, J=9.0 Hz, 1H), 6.70 (d, J=8.6 Hz, 1H), 6.61 (dd, J1=6.7 Hz, J2=5.0 Hz, 1H), 4.67 (s, 2H), 3.88 (dd, J1=12.2 Hz, J2=1.8 Hz, 2H), 3.83 (s, 3H), 3.05 (dd, J1=11.9 Hz, J2=1.5 Hz, 2H), 1.92 (t, J=4.0 Hz, 2H), 1.77 (q, J=5.7 Hz, 2H); ¹³C NMR, 100 MHz, DMSO-d6: δ=164.5, 161.8, 159.8, 153.9, 147.4, 140.8, 137.2, 131.3, 129.4, 126.7, 126.6, 113.5, 112.7, 108.7, 106.4, 55.4, 55.2, 48.0, 27.1; ESI-MS m/z calculated for C₂₄H₂₇N₅O₂+[M+2H]+417.5; found 417.5 HRMS (ESI) for C₂₄H₂₆N₅O₂+[M+H]+ 416.2087; found 416.2087

### Example 73: 4-methoxy-N-(4-(4-methylpiperazin-1-yl)phenyl)benzamide (113)

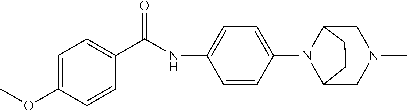

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(4-methylpiperazin-1-yl)phenyl)benzamide (5 mg, 8% yield) as a white solid. (1H NMR, 400 MHz, MeOD): δ=7.89 (dt, J1=8.8 Hz, J2=2.8 Hz, 2H), 7.53 (d, J=9.0 Hz, 2H), 7.02 (dt, J1=8.8 Hz, J2=2.9 Hz, 2H), 6.98 (dt, J1=9.0 Hz, J2=3.2 Hz, 2H), 3.86 (s, 3H), 3.19 (t, J=4.8 Hz, 4H), 2.63 (t, J=5.0 Hz, 4H), 2.35 (s, 3H) (¹³C NMR, 100 MHz, MeOD): δ=164.2, 149.8, 132.8, 132.4, 130.6, 128.4, 123.8, 117.9, 114.9, 56.14, 56.09, 50.5, 46.2; ESI-MS m/z calculated for C₁₉H₂₅N₃O₂+[M+2H]+327.4; found 327.4

### Example 74: N-(4-(1-azaspiro[3.5]nonan-1-yl)phenyl)-4-methoxybenzamide (114)

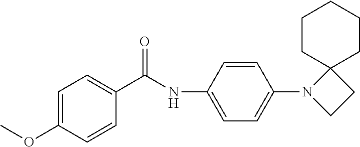 [SMILES: COc1ccc(C(=O)Nc2ccc(N3CCC34CCCCC4)cc2)cc1]

This compound was prepared as described herein. Purification by flash chromatography using 0% to 50% EtOAc/n-Hexane with gradient elution and CH₂Cl₂/7N NH₃ in MeOH=40:1 gave N-(4-(1-azaspiro[3.5]nonan-1-yl)phenyl)-4-methoxybenzamide (9 mg, 11% yield) as a white waxy solid. (1H NMR, 400 MHz, MeOD): δ=7.88 (dt, J1=8.8 Hz, J2=2.8 Hz, 2H), 7.40 (d, J=8.8 Hz, 2H), 7.01 (dt, J1=8.9 Hz, J2 2.9 Hz, 2H), 6.56 (dt, J1=8.9 Hz, J2=3.0 Hz, 2H), 3.86 (s, 3H), 3.65 (t, J=7.2 Hz, 2H), 2.10 (t, J=7.2 Hz, 2H), 1.92 (td, J1=12.8 Hz, J2=1.7 Hz, 2H), 1.80-1.71 (m, 4H), 1.41-1.28 (m, 3H), 1.20 (tt, J1=12.0 Hz, J2=4.1 Hz, 1H). ¹³C NMR, 100 MHz, MeOD: 6=164.1, 147.5, 130.5, 130.4, 128.5, 124.1, 115.1, 114.9, 72.2, 56.1, 46.5, 36.6, 29.6, 26.8, 24.4; ESI-MS m/z calculated for C₂₂H₂₈N₂O₂⁺ [M+2H]+352.5; found 352.5

### Example 75: 4-methoxy-N-(4-(3-methoxyazetidin-1-yl)phenyl)benzamide (115)

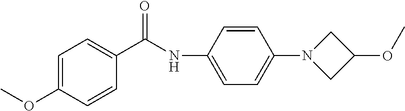

This compound was prepared as described herein. Purification by flash chromatography using 0% to 50% EtOAc/n-Hexane with gradient elution and CH₂Cl₂/7N NH₃ in MeOH=40:1 gave 4-methoxy-N-(4-(3-methoxyazetidin-1-yl)phenyl)benzamide (85 mg, 49% yield) as a white solid. (1H NMR, 400 MHz, DMSO-d6): δ=9.83 (s, 1H), 7.93 (dt, J1=8.8 Hz, J2=2.8 Hz, 2H), 7.54 (d, J=8.8 Hz, 2H), 7.04 (dt, J1=8.9 Hz, J2=2.8 Hz, 2H), 6.44 (dt, J1=8.8 Hz, J2=2.9 Hz, 2H), 4.33-4.28 (m, 1H), 4.03 (t, J=7.5 Hz, 2H), 3.83 (s, 3H), 3.55 (dd, J1=8.2 Hz, J2=4.5 Hz, 2H), 3.24 (s, 3H); ¹³C NMR, 100 MHz, DMSO-d6: δ=164.2, 161.6, 148.2, 129.8, 129.3, 127.2, 121.8, 113.5, 111.4, 69.3, 58.8, 55.4, 55.3; ESI-MS m/z calculated for C₁₈H₂₂N₂O₃⁺ [M+2H]+ 314.4; found 314.4

### Example 76: 4-(2-fluoroethoxy)-N-(4-(3-(pyridin-2-yl)-3,8-diazabicyclo[3.2.1]octan-8-yl)phenyl)benzamide (116)

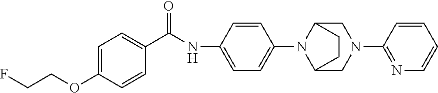 [SMILES: O=C(Nc1ccc(N2C3CCC2CN(c2ccccn2)C3)cc1)c1ccc(OCCF)cc1]

This compound was prepared as described herein. Purification by flash chromatography using 0% to 50% EtOAc/n-Hexane with gradient elution and CH₂Cl₂/7N NH₃ in MeOH=40:1 gave 4-(2-fluoroethoxy)-N-(4-(3-(pyridin-2-yl)-3,8-diazabicyclo[3.2.1]octan-8-yl)phenyl)benzamide (10 mg, 72% yield) as a white solid. (1H NMR, 400 MHz, DMSO-d6): δ=9.84 (s, 1H), 8.07 (dd, J1=4.8 Hz, J2=1.4 Hz, 1H), 7.93 (d, J=8.8 Hz, 2H), 7.58 (d, J=9.0 Hz, 2H), 7.49 (ddd, J1=8.7 Hz, J2=7.0 Hz, J3=2.0 Hz, 1H), 7.07 (d, J=8.8 Hz, 2H), 6.92 (d, J=8.6 Hz, 2H), 6.68 (d, J=8.6 Hz, 1H), 6.61 (dd, J1=6.9 Hz, J2=5.0 Hz, 1H), 4.83 (t, J=3.8 Hz, 1H), 4.71 (t, J=3.8 Hz, 1H), 4.40 (s, 2H), 4.36 (t, J=3.8 Hz, 1H), 4.28 (t, J=3.9 Hz, 1H), 3.76 (d, J=11.0 Hz, 2H), 3.11 (d, J=10.8 Hz, 2H), 1.95 (t, J=3.9 Hz, 2H), 1.77 (q, J=5.9 Hz, 2H); ¹³C NMR, 100 MHz, DMSO-d6: δ=164.1, 160.5, 159.9, 147.4, 143.0, 137.2, 129.8, 129.3, 127.6, 122.0, 115.5, 114.0, 112.7, 106.3, 82.8, 81.2, 67.3, 67.1, 53.9, 46.8, 27.2; ESI-MS m/z calculated for C₂₆H₂₉FN₄O₂⁺ [M+2H]+448.5; found 448.6 HRMS (ESI) for C₂₆H₂₈FN₄O₂⁺ [M+H]+ 447.2196; found 447.2183

### Example 77: Ethyl 4-amino-2-(4-(pyridin-4-yl)piperazin-1-yl)pyrimidine-5-carboxylate (117)

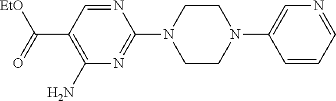 [SMILES: CCOC(=O)c1cnc(N2CCN(c3cccnc3)CC2)nc1N]

This compound was prepared using the procedures described herein. ¹H NMR (400 MHz, DMSO) δ 8.53 (s, 1H), 8.34 (d, J=2.9 Hz, 1H), 8.02 (dd, J=4.5, 1.3 Hz, 1H), 7.46 (d, J=13.0 Hz, 2H), 7.36 (ddd, J=8.5, 3.0, 1.4 Hz, 1H), 7.23 (dd, J=8.5, 4.5 Hz, 1H), 4.22 (q, J=7.1 Hz, 2H), 3.93 (t, J=5.1 Hz, 4H), 3.25 (t, J=5.2 Hz, 4H), 1.27 (t, J=7.1 Hz, 3H); 13C NMR (101 MHz, DMSO) δ 165.9, 162.8, 161.3, 160.5, 146.6, 140.0, 138.1, 123.5, 122.1, 95.0, 59.7, 47.5, 42.8, 14.2. LCMS: 329 [M+H]+; HRMS (ESI) calculated for C₁₆H₂₁N₆O₂⁺ ([M+H+]) 329.1721, found: 329.1726. White solid, 55 mg, 56%.

### Example 78: 4-Amino-N-(4-methoxyphenyl)-2-(4-(pyridin-3-yl)piperazin-1-yl)pyrimidine-5-carboxamide (118)

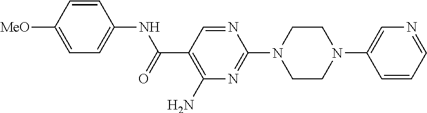 [SMILES: COc1ccc(NC(=O)c2cnc(N3CCN(c4cccnc4)CC3)nc2N)cc1]

This compound was prepared using the procedures described herein. ¹H NMR (400 MHz, DMSO) δ 9.74 (s, 1H), 8.62 (s, 1H), 8.35 (d, J=2.9 Hz, 1H), 8.02 (d, J=4.4 Hz, 1H), 7.54 (d, J=9.1 Hz, 3H), 7.37 (ddd, J=8.6, 3.0, 1.4 Hz, 1H), 7.23 (dd, J=8.5, 4.5 Hz, 1H), 6.89 (d, J=9.1 Hz, 2H), 3.92 (t, J=5.2 Hz, 4H), 3.73 (s, 3H), 3.25 (t, J=5.3 Hz, 4H); ¹³C NMR (101 MHz, DMSO) δ 165.3, 162.9, 160.8, 157.4, 155.4, 146.7, 140.0, 138.1, 132.0, 123.5, 122.2, 122.1, 113.7, 99.1, 55.2, 47.6, 42.8. LCMS: 406 [M+H]+; HRMS (ESI) calculated for C₂₁H₂₄N₇O₂⁺ ([M+H+]) 406.1986, found: 406.1998. White solid, 32 mg, 39%.

### Example 79: Ethyl 4-amino-2-(4-(pyrimidin-2-yl)piperazin-1-yl)pyrimidine-5-carboxylate (119)

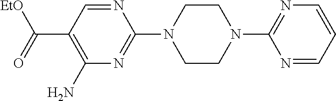 [SMILES: CCOC(=O)c1cnc(N2CCN(c3ncccn3)CC2)nc1N]

This compound was prepared using the procedures described herein. ¹H NMR (400 MHz, DMSO) δ 8.53 (s, 1H), 8.38 (d, J=4.7 Hz, 2H), 7.46 (d, J=9.5 Hz, 2H), 6.65 (t, J=4.7 Hz, 1H), 4.22 (q, J=7.1 Hz, 2H), 3.86 (dd, J=6.9, 3.8 Hz, 4H), 3.79 (dd, J=7.1, 3.9 Hz, 4H), 1.27 (t, J=7.1 Hz, 3H); ¹³C NMR (101 MHz, DMSO) δ 165.9, 162.8, 161.4, 161.1, 160.4, 157.9, 110.3, 95.0, 59.7, 43.1, 43.0, 14.2. LCMS: 330 [M+H]+; HRMS (ESI) calculated for C₁₅H₂₀N₇O₂⁺ ([M+H+]) 330.1673, found: 330.1667. White solid, 67 mg, 68%.

### Example 80: 4-Amino-N-(4-methoxyphenyl)-2-(4-(pyrimidin-2-yl)piperazin-1-yl)pyrimidine-5-carboxamide (120)

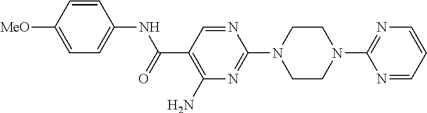 [SMILES: COc1ccc(NC(=O)c2cnc(N3CCN(c4ncccn4)CC3)nc2N)cc1]

This compound was prepared using the procedures described herein. ¹H NMR (400 MHz, DMSO) δ 9.74 (s, 1H), 8.61 (s, 1H), 8.39 (d, J=4.8 Hz, 2H), 7.53 (d, J=9.1 Hz, 3H), 6.89 (d, J=9.0 Hz, 2H), 6.66 (t, J=4.7 Hz, 1H), 3.85 (d, J=4.9 Hz, 4H), 3.82-3.77 (m, 4H), 3.73 (s, 3H); ¹³C NMR (101 MHz, DMSO) δ 165.8, 163.4, 161.7, 161.4, 158.5, 157.9, 155.9, 132.5, 122.8, 114.1, 110.8, 99.5, 55.6, 43.6, 43.4. LCMS: 407 [M+H]; HRMS (ESI) calculated for C₂₀H₂₃N₈O₂⁺ ([M+H+]) 407.1938, found: 407.1968. White solid, 47 mg, 58%.

### Example 81: Ethyl 4-amino-2-(4-(pyrazin-2-yl)piperazin-1-yl)pyrimidine-5-carboxylate (121)

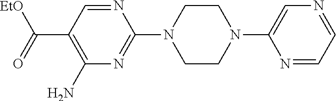 [SMILES: CCOC(=O)c1cnc(N2CCN(c3cnccn3)CC2)nc1N]

This compound was prepared using the procedures described herein. ¹H NMR (400 MHz, DMSO) δ 8.53 (s, 1H), 8.36 (d, J=1.6 Hz, 1H), 8.10 (dd, J=2.6, 1.5 Hz, 1H), 7.86 (d, J=2.6 Hz, 1H), 7.47 (d, J=7.2 Hz, 2H), 4.22 (q, J=7.1 Hz, 2H), 3.91-3.88 (m, 4H), 3.67-3.62 (m, 4H), 1.27 (t, J=7.1 Hz, 3H); ¹³C NMR (101 MHz, DMSO) δ 165.9, 162.8, 161.3, 160.5, 154.5, 141.4, 132.6, 131.4, 95.0, 59.7, 43.7, 42.7, 14.2. LCMS: 330 [M+H]+; HRMS (ESI) calculated for C₁₅H₂₀N₇O₂⁺ ([M+H+]) 330.1673, found: 330.1693. White solid, 70 mg, 71%.

### Example 82: 4-Amino-N-(4-methoxyphenyl)-2-(4-(pyrazin-2-yl)piperazin-1-yl)pyrimidine-5-carboxamide (122)

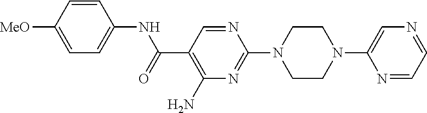 [SMILES: COc1ccc(NC(=O)c2cnc(N3CCN(c4cnccn4)CC3)nc2N)cc1]

This compound was prepared using the procedures described herein. ¹H NMR (400 MHz, DMSO) δ 9.75 (s, 1H), 8.62 (s, 1H), 8.37 (d, J=1.6 Hz, 1H), 8.11 (dd, J=2.6, 1.5 Hz, 1H), 7.86 (d, J=2.6 Hz, 1H), 7.54 (d, J=9.1 Hz, 3H), 6.89 (d, J=9.1 Hz, 2H), 3.93-3.86 (m, 4H), 3.73 (s, 3H), 3.68-3.62 (m, 4H); ¹³C NMR (101 MHz, DMSO) δ 165.3, 162.9, 160.8, 157.4, 155.4, 154.6, 141.4, 132.6, 132.0, 131.4, 122.3, 113.7, 99.1, 55.1, 43.7, 42.7. LCMS: 407 [M+H]⁺; HRMS (ESI) calculated for C₂₀H₂₃N₈O₂⁺ ([M+H+]) 407.1938, found: 407.1956. White solid, 45 mg, 55%.

### Example 83: Ethyl 4-amino-2-(4-(thiazol-2-yl)piperazin-1-yl)pyrimidine-5-carboxylate (123)

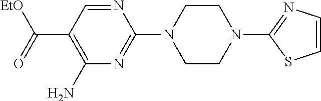 [SMILES: CCOC(=O)c1cnc(N2CCN(c3nccs3)CC2)nc1N]

This compound was prepared using the procedures described herein. ¹H NMR (400 MHz, DMSO) δ 8.53 (s, 1H), 7.47 (d, J=5.8 Hz, 2H), 7.18 (d, J=3.6 Hz, 1H), 6.87 (d, J=3.6 Hz, 1H), 4.22 (q, J=7.1 Hz, 2H), 3.91 (t, J=5.2 Hz, 4H), 3.50-3.43 (m, 4H), 1.27 (t, J=7.1 Hz, 3H); ¹³C NMR (101 MHz, DMSO) δ 171.3, 165.9, 162.8, 161.3, 160.5, 139.4, 108.3, 95.2, 59.7, 48.0, 42.4, 14.2. LCMS: 335 [M+H]⁺; HRMS (ESI) calculated for C₁₄H₁₉N₆O₂S⁺ ([M+H+]) 335.1285, found: 335.1304. White solid, 57 mg, 57%.

### Example 84: 4-Amino-N-(4-methoxyphenyl)-2-(4-(thiazol-2-yl)piperazin-1-yl)pyrimidine-5-carboxamide (124)

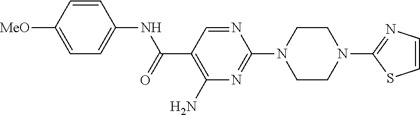 [SMILES: COc1ccc(NC(=O)c2cnc(N3CCN(c4nccs4)CC3)nc2N)cc1]

This compound was prepared using the procedures described herein. ¹H NMR (400 MHz, DMSO) δ 9.75 (s, 1H), 8.61 (s, 1H), 7.53 (d, J=9.0 Hz, 3H), 7.19 (d, J=3.6 Hz, 1H), 6.93-6.85 (m, 3H), 3.91 (t, J=5.3 Hz, 4H), 3.73 (s, 3H), 3.47 (t, J=5.2 Hz, 4H); ¹³C NMR (101 MHz, DMSO) δ 171.4, 165.3, 162.9, 160.8, 157.4, 155.4, 139.4, 132.0, 122.3, 113.7, 108.3, 99.2, 55.2, 48.1, 42.4. LCMS: 412 [M+H]+; HRMS (ESI) calculated for C₁₉H₂₂N₇O₂S⁺ ([M+H+]) 412.1550, found: 412.1566. White solid, 57 mg, 70%.

### Example 85: Ethyl 4-amino-2-(3-(benzo[d]thiazol-2-yl)-3,8-diazabicyclo[3.2.1]octan-8-yl)pyrimidine-5-carboxylate (125)

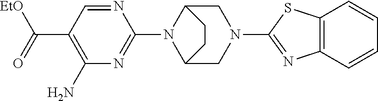 [SMILES: CCOC(=O)c1cnc(N2C3CCC2CN(c2nc4ccccc4s2)C3)nc1N]

This compound was prepared using the procedures described herein. ¹H NMR (400 MHz, DMSO-d6) δ 8.55 (s, 1H), 7.75 (dd, J=7.9, 1.2 Hz, 1H), 7.51 (s, 2H), 7.45 (d, J=7.5 Hz, 1H), 7.27 (td, J=7.7, 1.3 Hz, 1H), 7.06 (td, J=7.6, 1.2 Hz, 1H), 4.88 (d, J=22.3 Hz, 2H), 4.22 (q, J=7.1 Hz, 2H), 3.85 (d, J=12.0 Hz, 2H), 3.36 (d, J=12.0 Hz, 2H), 1.98-1.94 (m, 2H), 1.81 (q, J=6.7 Hz, 2H), 1.27 (t, J=7.1 Hz, 3H); 13C NMR (101 MHz, DMSO-d6) δ 169.0, 165.9, 163.1, 160.8, 159.9, 152.3, 130.0, 126.0, 121.1, 121.1, 118.5, 95.6, 59.7, 52.4, 26.7, 14.2. LCMS: 411 [M+H]+; HRMS (ESI) calculated for C20H23N6O2S+([M+H+]) 411.1598, found: 411.1605. White solid, 87 mg, 71%.

### Example 86: 4-Amino-N-(4-methoxyphenyl)-2-(8-(quinolin-3-yl)-3,8-diazabicyclo[3.2.1]octan-3-yl)pyrimidine-5-carboxamide (126)

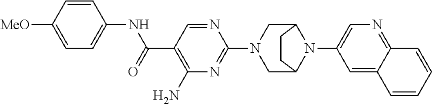 [SMILES: COc1ccc(NC(=O)c2cnc(N3CC4CCC(C3)N4c3cnc4ccccc4c3)nc2N)cc1]

¹H NMR (400 MHz, DMSO-d6) δ 9.73 (s, 1H), 8.87 (d, J=2.9 Hz, 1H), 8.57 (s, 1H), 7.85 (dd, J=7.7, 1.8 Hz, 1H), 7.76-7.71 (m, 1H), 7.61 (d, J=2.8 Hz, 1H), 7.53 (d, J=9.0 Hz, 2H), 7.47-7.43 (m, 2H), 6.89 (d, J=9.0 Hz, 2H), 4.64 (s, 2H), 4.42 (d, J=13.0 Hz, 2H), 3.73 (s, 3H), 3.24 (d, J=12.9 Hz, 2H), 1.99 (d, J=11.5 Hz, 3H), 1.73 (d, J=7.4 Hz, 2H); ¹³C NMR (101 MHz, DMSO-d6) δ 165.8, 163.1, 162.7, 157.7, 155.8, 144.2, 141.9, 140.8, 132.6, 129.6, 128.9, 127.2, 126.7, 125.7, 122.7, 116.0, 114.1, 99.6, 55.6, 54.6, 46.6, 27.1. LCMS: 482 [M+H]+; HRMS (ESI) calculated for C₂₇H₂₈N₇O₂₊ ([M+H+]) 482.2299, found: 482.2299. Light-yellow solid, 42 mg, 44% for 2 steps.

### Example 87: 4-amino-N-(2-fluoro-4-methoxyphenyl)-2-(4-(pyridin-2-yl)piperazin-1-yl)pyrimidine-5-carboxamide (127)

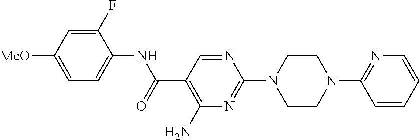 [SMILES: COc1ccc(NC(=O)c2cnc(N3CCN(c4ccccn4)CC3)nc2N)c(F)c1]

¹H NMR (400 MHz, DMSO) δ 9.58 (s, 1H), 8.65 (s, 1H), 8.13 (dd, J=5.3, 1.9 Hz, 1H), 7.58-7.54 (m, 3H), 7.32 (t, J=8.9 Hz, 1H), 6.96-6.83 (m, 2H), 6.77 (dd, J=8.9, 2.3 Hz, 1H), 6.66 (dd, J=7.1, 4.9 Hz, 1H), 3.87 (t, J=5.3 Hz, 4H), 3.77 (s, 3H), 3.56 (t, J=5.3 Hz, 4H); ¹³C NMR (101 MHz, DMSO) δ 165.8, 162.9, 160.9, 158.9, 158.2 (d, J=228.4 Hz), 157.8, 156.9 (d, J=7.3 Hz), 147.6, 137.6, 128.6 (d, J=3.4 Hz), 118.0 (d, J=13.0 Hz), 113.2, 109.7 (d, J=3.1 Hz), 107.2, 101.9 (d, J=23.9 Hz), 98.2, 55.7, 44.4, 42.9. LCMS: 424 [M+H]+; HRMS (ESI) calculated for C₂₁H₂₃FN₇O₂⁺ ([M+H+]) 424.1892, found: 424.1887. White solid, 42 mg, 50%.

### Example 88: 4-amino-N-(3-fluoro-4-methoxyphenyl)-2-(4-(pyridin-2-yl)piperazin-1-yl)pyrimidine-5-carboxamide (128)

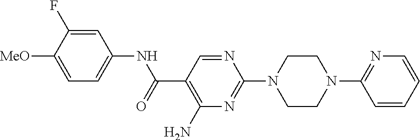 [SMILES: COc1ccc(NC(=O)c2cnc(N3CCN(c4ccccn4)CC3)nc2N)cc1F]

¹H NMR (400 MHz, DMSO) δ 9.86 (s, 1H), 8.61 (s, 1H), 8.13 (dd, J=4.9, 1.7 Hz, 1H), 7.70-7.47 (m, 4H), 7.36 (ddd, J=9.0, 2.6, 1.4 Hz, 1H), 7.12 (t, J=9.3 Hz, 1H), 6.87 (d, J=8.6 Hz, 1H), 6.66 (dd, J=7.1, 4.9 Hz, 1H), 3.90-3.84 (m, 4H), 3.81 (s, 3H), 3.60-3.54 (m, 4H); ¹³C NMR (101 MHz, DMSO) δ 165.5, 162.8, 160.9, 158.9, 159.9, 157.6, 154.9 (d, J=3.0 Hz), 151.9, 147.5, 137.6, 116.4 (d, J=3.0 Hz), 113.8, 113.2, 109.0, 107.2, 98.8, 56.2, 44.4, 42.9.LCMS: 424 [M+H]⁺; HRMS (ESI) calculated for C₂₁H₂₃FN₇O₂⁺ ([M+H+]) 424.1892, found: 424.1906. White solid, 45 mg, 53%.

### Example 89: 2-Hydroxyethyl 4-amino-2-(4-(pyridin-2-yl)piperazin-1-yl)pyrimidine-5-carboxylate (129)

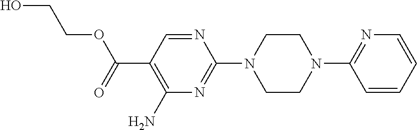 [SMILES: Nc1nc(N2CCN(c3ccccn3)CC2)ncc1C(=O)OCCO]

¹H NMR (400 MHz, DMSO-d6) δ 8.61 (s, 1H), 8.13 (dd, J=5.0, 2.0 Hz, 1H), 7.55 (ddd, J=8.9, 7.0, 2.0 Hz, 1H), 7.44 (s, 2H), 6.86 (d, J=8.6 Hz, 1H), 6.66 (dd, J=7.1, 4.9 Hz, 1H), 4.89 (t, J=5.7 Hz, 1H), 4.18 (t, J=5.7 Hz, 2H), 3.90-3.87 (m, 4H), 3.66 (q, J=5.4 Hz, 2H), 3.59-3.50 (m, 4H); ¹³C NMR (101 MHz, DMSO-d6) δ 166.0, 162.8, 161.4, 160.8, 158.8, 147.6, 137.6, 113.2, 107.2, 94.9, 65.6, 59.1, 44.4, 42.9. LCMS: 345 [M+H]+; HRMS (ESI) calculated for C₁₆H₂₁N₆O₃ ([M+H+]) 345.1670, found: 345.1677. White solid, 28 mg, 36% for 2 steps.

### Example 90: N-(4-Methoxyphenyl)-2-(4-(pyridin-2-yl)piperazin-1-yl)pyrimidine-5-carboxamide (130)

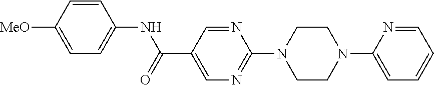 [SMILES: COc1ccc(NC(=O)c2cnc(N3CCN(c4ccccn4)CC3)nc2)cc1]

¹H NMR (400 MHz, DMSO-d6) δ 9.99 (s, 1H), 8.91 (s, 2H), 8.14 (dd, J=4.9, 2.0 Hz, 1H), 7.62 (d, J=9.0 Hz, 2H), 7.59-7.54 (m, 1H), 6.92 (d, J=9.0 Hz, 2H), 6.88 (d, J=8.7 Hz, 1H), 6.67 (dd, J=7.1, 4.9 Hz, 1H), 3.96 (t, J=5.3 Hz, 4H), 3.74 (s, 3H), 3.62 (t, J=5.3 Hz, 4H); ¹³C NMR (101 MHz, DMSO-d6) δ 162.2, 161.4, 158.8, 158.0, 155.5, 147.6, 137.7, 132.0, 121.9, 116.7, 113.8, 113.3, 107.3, 55.2, 44.3, 43.2. LCMS: 391 [M+H]+; HRMS (ESI) calculated for C₂₁H₂₃N₆O₂⁺ ([M+H+]) 391.1877, found: 391.1879. White solid, 59 mg, 76%.

### Example 91: 4-Amino-N-(4-hydroxyphenyl)-2-(4-(pyridin-2-yl)piperazin-1-yl)pyrimidine-5-carboxamide (131)

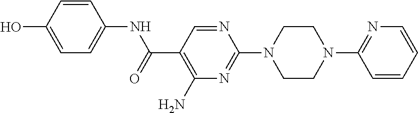 [SMILES: Nc1nc(N2CCN(c3ccccn3)CC2)ncc1C(=O)Nc1ccc(O)cc1]

¹H NMR (400 MHz, DMSO-d6) δ 9.66 (s, 1H), 9.22 (s, 1H), 8.59 (s, 1H), 8.13 (ddd, J=5.0, 2.0, 0.8 Hz, 1H), 7.56 (ddd, J=8.9, 7.1, 2.0 Hz, 1H), 7.39 (d, J=8.9 Hz, 2H), 6.87 (d, J=8.6 Hz, 1H), 6.71 (d, J=8.9 Hz, 2H), 6.66 (ddd, J=7.2, 4.8, 0.8 Hz, 1H), 3.89-3.83 (m, 4H), 3.59-3.52 (m, 4H); ¹³C NMR (101 MHz, DMSO-d6) δ 165.2, 162.9, 160.9, 158.9, 157.3, 153.5, 147.6, 137.6, 130.5, 122.6, 114.9, 113.2, 107.3, 99.1, 44.5, 42.9. LCMS: 392 [M+H]+. White solid, 41 mg, 53%.

### Example 92: 4-Amino-N-(4-(2-fluoroethoxy)phenyl)-2-(4-(pyridin-2-yl)piperazin-1-yl)pyrimidine-5-carboxamide (132)

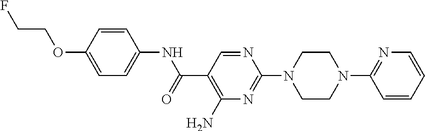 [SMILES: Nc1nc(N2CCN(c3ccccn3)CC2)ncc1C(=O)Nc1ccc(OCCF)cc1]

¹H NMR (400 MHz, DMSO-d6) δ 9.76 (s, 1H), 8.61 (s, 1H), 8.13 (ddd, J=4.9, 2.0, 0.8 Hz, 1H), 7.57-7.53 (m, 3H), 6.93 (d, J=9.1 Hz, 2H), 6.87 (d, J=8.6 Hz, 1H), 6.66 (ddd, J=7.1, 4.9, 0.8 Hz, 1H), 4.81-4.77 (m, 1H), 4.69-4.65 (m, 1H), 4.26-4.22 (m, 1H), 4.18-4.14 (m, 1H), 3.90-3.84 (m, 4H), 3.59-3.54 (m, 4H); ¹³C NMR (101 MHz, DMSO-d6) δ 165.4, 162.9, 160.9, 158.9, 157.5, 154.2, 147.6, 137.6, 132.4, 122.2, 114.4, 113.2, 107.2, 99.0, 82.2 (d, J=166.7 Hz), 67.2 (d, J=19.0 Hz), 44.4, 42.9. LCMS: 438 [M+H]+; HRMS (ESI) calculated for C₂₂H₂₅FN₇O₂⁺ ([M+H+]) 438.2048, found: 438.2044. White solid, 30 mg, 68%.

### Example 93: 4-Amino-2-(4-(benzo[d]oxazol-2-yl)piperazin-1-yl)-N-(4-(2-fluoroethoxy)phenyl)pyrimidine-5-carboxamide (133)

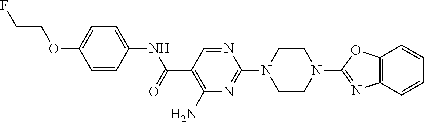 [SMILES: Nc1nc(N2CCN(c3nc4ccccc4o3)CC2)ncc1C(=O)Nc1ccc(OCCF)cc1]

¹H NMR (400 MHz, DMSO-d6) δ 9.78 (s, 1H), 8.63 (s, 1H), 7.65-7.49 (m, 3H), 7.42 (d, J=8.0 Hz, 1H), 7.31 (d, J=7.7 Hz, 1H), 7.17 (td, J=7.7, 1.2 Hz, 1H), 7.04 (td, J=7.7, 1.2 Hz, 1H), 6.93 (d, J=9.0 Hz, 2H), 4.81-4.76 (m, 1H), 4.69-4.65 (m, 1H), 4.26-4.22 (m, 1H), 4.19-4.15 (m, 1H), 3.98-3.90 (m, 4H), 3.69-3.67 (m, 4H); ¹³C NMR (101 MHz, DMSO-d6) δ 165.3, 162.9, 161.8, 160.8, 157.5, 154.2, 148.3, 142.9, 132.4, 124.0, 122.3, 120.6, 115.9, 114.4, 108.9, 99.3, 82.2 (d, J=166.4 Hz), 67.2 (d, J=19.0 Hz), 45.0, 42.5. LCMS: 478 [M+H]+; HRMS (ESI) calculated for C₂₄H₂₅FN₇O₃⁺ ([M+H+]) 478.1997, found: 478.2007. White solid, 34 mg, 72%.

### Example 94: 4-Amino-N-(4-(3-fluoro-2-hydroxypropoxy)phenyl)-2-(4-(pyridin-2-yl)piperazin-1-yl)pyrimidine-5-carboxamide (134), (S)-4-amino-N-(4-(3-fluoro-2-hydroxypropoxy)phenyl)-2-(4-(pyridin-2-yl)piperazin-1-yl)pyrimidine-5-carboxamide (135), (R)-4-amino-N-(4-(3-fluoro-2-hydroxypropoxy)phenyl)-2-(4-(pyridin-2-yl)piperazin-1-yl)pyrimidine-5-carboxamide (136)

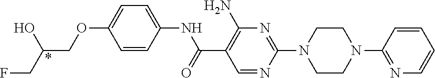 [SMILES: Nc1nc(N2CCN(c3ccccn3)CC2)ncc1C(=O)Nc1ccc(OCC(O)CF)cc1]

¹H NMR (400 MHz, DMSO-d6) δ 9.75 (s, 1H), 8.61 (s, 1H), 8.13 (ddd, J=4.9, 2.0, 0.8 Hz, 1H), 7.59-7.51 (m, 4H), 6.93-6.85 (m, 3H), 6.68-6.64 (m, 1H), 5.42 (d, J=5.2 Hz, 1H), 4.59-4.38 (m, 2H), 4.04-3.93 (m, 3H), 3.90-3.83 (m, 4H), 3.59-3.52 (m, 4H); ¹³C NMR (101 MHz, DMSO-d6) δ 165.4, 162.9, 160.9, 158.9, 157.4, 154.5, 147.6, 137.6, 132.3, 122.2, 114.4, 113.2, 107.2, 99.0, 84.4 (d, J=168.2 Hz), 68.3 (d, J=7.9 Hz), 67.7 (d, J=19.0 Hz), 44.4, 42.9. 134: LCMS: 468 [M+H]+; HRMS (ESI) calculated for C₂₃H₂₇FN₇O₃⁺ ([M+H+]) 468.2154, found: 468.2155. White solid, 24 mg, 53%; 135: LCMS: 468 [M+H]+; HRMS (ESI) calculated for C₂₃H₂₇FN₇O₃⁺ ([M+H+]) 468.2154, found: 468.2155. White solid, 28 mg, 61%; 136: LCMS: 468 [M+H]+; HRMS (ESI) calculated for C₂₃H₂₇FN₇O₃⁺ ([M+H+]) 468.2154, found: 468.2185. White solid, 27 mg, 58%.

### Example 95: Assays

Competition binding assays were performed with [³H]BF2846 and [³H]PIB to measure affinity to Asyn and Aβ. See, Table 1.

TABLE 1

| Compound | α-syn Kᵢ (nM) | AD Kᵢ (nM) |
|---|---|---|
| 1 | 11.6 | NA |
| 2 | 26.3 | NA |
| 3 | 23.3 | NA |
| 4 | 5.5 | NA |
| 5 | 58.5 | NA |
| 6 | 9.9 | NA |
| 7 | 8.9 | NA |
| 8 | 15.2 | NA |
| 9 | 4 | NA |
| 10 | 2.5 | 135 |
| 11 | 12.9 | NA |
| 12 | 23.4 | NA |
| 13 | 12.1 | NA |
| 14 | 3.1 | 124 |
| 15 | 10.8 | 661 |
| 16 | 2.4 | 89 |
| 17 | 12.8 | 142 |
| 18 | NA | NA |
| 19 | 45 | ND |
| 20 | NA | NA |
| 21 | 255 | NA |
| 23 | NA | NA |
| 24 | NA | NA |
| 25 | NA | NA |
| 26 | 6 | NA |
| 27 | 1.1 | NA |
| 29 | 7.7 | NA |
| 30 | 4.4 | NA |
| 31 | 1.6 | NA |
| 32 | 6.9 | 23 |
| 33 | 44.5 | NA |
| 34 | 10 | ND |
| 35 | NA | NA |
| 36 | NA | NA |
| 37 | 41.4 | ND |
| 38 | NA | NA |
| 39 | 84.12 | NA |
| 40 | NA | NA |
| 41 | 7.5 | 236 |
| 42 | 6.8 | 147 |
| 43 | 6.5 | 390 |
| 117 | NA | NA |
| 118 | NA | NA |
| 119 | NA | NA |
| 120 | NA | NA |
| 100 | NA | NA |
| 101 | 21.9 ± 9.7 | NA |
| 102 | NA | NA |
| 103 | 6.1 ± 2.1 | NA |
| 104 | NA | NA |
| 105 | 47.6 ± 14 | NA |
| 106 | NA | NA |
| 107 | 8.7 ± 0.6 | NA |
| 108 | NA | NA |
| 109 | 5.0 ± 0.34 | 42.9 ± 3.8 |
| 110 | 44.1 ± 32 | NA |
| 111 | NA | NA |
| 112 | NA | NA |
| 113 | NA | NA |
| 114 | NA | NA |
| 115 | 28.6 ± 33 | NA |
| 116 | 12.7 ± 2.3 | NA |
| 117 | 42.1 | 197.4 |
| 118 | 142.3 | ND |
| 119 | 19.5 | ND |
| 120 | 41.9 | ND |
| 121 | 8.4 | 40.9 |
| 122 | 13.5 | 297 |
| 123 | 51.1 | 116.8 |
| 124 | 29.9 | NA |
| 125 | 23.4 | >1,000 |
| 126 | 522 | NA |
| 127 | 51.7 | NA |
| 128 | 33.3 | 275.4 |
| 129 | 41.4 | ND |
| 130 | 6.8 | 147 |
| 131 | ND | ND |
| 132 | 4.9 | ND |
| 133 | 1.26 | ND |
| 134 | 27.6 | ND |
| 135 | 16.9 | ND |
| 136 | 24.8 | ND |

Kᵢ values (nM) were determined from competitive radioligand binding assay using α-synuclein fibrils with [³H]BF2846 and AD CAA homogenage with [³H]PIB. The Kᵢ values are expressed as the mean + Sd. for n = 3. NA = non active. ND = not determined.

K_{d} and Bₘₐₓ values of [³H]-Compound 113 were obtained from a panel of postmortem brain samples (e.g., PD, MSA, corticobasal degeneration (CBD), progressive supranuclear palsy (PSP), Alzheimer's disease (AD)). Nuclear emulsion autoradiography identified the distribution of [³H]-Compound 113 on PD and MSA tissues for aggregated Asyn, which was confirmed by antibody staining. Dynamic whole body PET images of [¹¹C]-Compound 113 in non-human primate were obtained over 90 min. In in vitro binding assays, Compound 113 exhibited high binding affinity for Asyn (6.1±2.1 nM) and low binding affinity at Aβ fibrils (33-115 nM). In the tissue binding study, [³H]-Compound 113 exhibited high specific binding to all the investigated tissues (K_{d}=5.1 nM for PD tissue; 4.5 nM for MSA; 18 nM for CBD, and 7.1 nM for PSP and 3.2 for AD). Interestingly, [³H]-Compound 113 had higher binding to pathologic Asyn in MSA compared to PD brain sections in autoradiography, suggesting [³H]-Compound 113 may be specific for MSA Asyn. In AD tissue, Compound 113 competed poorly with [³H]PiB (Ki=120 nM) for Aβ, indicating high affinity binding of Compound 113 to AD tau only. The high affinity of [³H]-Compound 113 to AD, CBD and PSP may reflect binding to a novel binding site on aggregated tau since it could not be blocked with the tau ligands PI-2620, JNJ-067, PM-PBB3, and GTP-1 in homogenate binding and autoradiography studies. The non-human primate PET study confirmed that [¹¹C]-Compound 113 had good brain uptake (SUV ~1.6) and rapid washout (2:60 min ratio ~3.0).

Thus, [¹¹C]-Compound 113 displayed binding to Asyn fibrils with low nM binding affinity. Tissue binding and autoradiography studies demonstrated [³H]-Compound 113 can bind to Asyn in postmortem tissue, particularly Asyn aggregates in MSA. Binding in CBD and PSP may indicate binding to a new site in aggregated tau. Whole body PET confirmed the favorable in vivo properties of [¹¹C]-Compound 113 for brain imaging.

The contents of all references, patent applications, patents, and published patent applications, as well as the Figures, cited throughout this application are hereby incorporated by reference.

Those skilled in the art will recognize, or be able to ascertain using no more than routine experimentation, many equivalents to the specific embodiments of the disclosure described herein. Such equivalents are intended to be encompassed by the following claims.

1. A compound of Formula I or II:
(a) 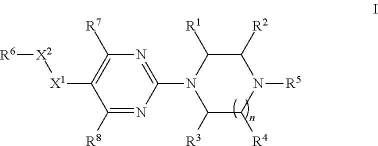
wherein:
n is 1 or 2;
R¹ to R⁴ are H; or R¹ and R³ are H and R² and R⁴ are joined to form —C₁₋₆alk-; or R² and R⁴ are H and R¹ and R³ are joined to form —C₁₋₆alk-;
R⁵ is optionally substituted aryl or optionally substituted heteroaryl;
X¹ is absent or —C(O)—;
X² absent, O, or NH;
R⁶ is H, CN, C₁₋₆alkyl, C₁₋₆alk-OH, or optionally substituted phenyl; and
R⁷ and R⁸ are, independently, H, halo, or NH₂; or
(b) 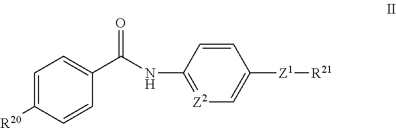
wherein:
R²⁰ is optionally substituted C₁₋₆alkoxy or optionally substituted C₁₋₆haloalkoxy;
R²¹ is H, optionally substituted C₁₋₆alkyl, optionally substituted C₁₋₆alkoxy, optionally substituted heteroaryl or optionally substituted aryl;
Z¹ is 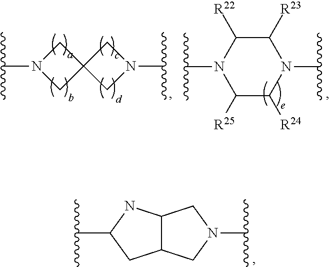
or 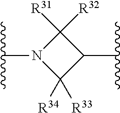
Z² is CH or N;
a, b, c, d, and e are, independently, 1 or 2;
R²², R²³, R²⁴, and R²⁵ are H; or R²³ and R²⁴ are H and R²² and R²⁵ are joined to form —C₁₋₆alk-; or R²³ and R²⁵ are H and R²² and R²⁴ are joined to form —C₁₋₆alk-; or R²² and R²⁴ are H and R²³ and R²⁵ are joined to form —C₁₋₆alk-; or R²² and R²⁵ are H and R²³ and R²⁴ are joined to form —C₁₋₆alk-;
R³¹, R³², R³³, and R³⁴ are H; or R³³ and R³⁴ are H and R³¹ and R³² are joined to form a C₃₋₈cycloalkyl; or R³¹ and R³² are H and R³³ and R³⁴ are joined to form a C₃₋₈cycloalkyl:
or a pharmaceutically acceptable salt or isotopic variant thereof.

2-3. (canceled)

4. The compound of claim 1, wherein R¹ to R⁴, R⁶, R⁷, or R²¹ are H.

5. The compound of claim 1, wherein R¹ and R³ are H and R² and R⁴ are joined to form —C₁₋₆alk.

6. The compound of claim 1, wherein R⁵ or R²¹ is optionally substituted aryl.

7. The compound of claim 1, wherein R⁵ or R²¹ is optionally substituted heteroaryl.

8. The compound of claim 1, wherein X¹ and/or X² is absent.

9. The compound of claim 1, wherein X¹ is —C(O)—.

10. The compound of claim 1, wherein X² is O.

11. The compound of claim 1, wherein X² is NH.

12. (canceled)

13. The compound of claim 1, wherein R⁶ is CN.

14. The compound of claim 1, wherein R⁶ or R²¹ is C₁₋₆alkyl.

15. The compound of claim 1, wherein R⁶ is C₁₋₆alkOH.

16. The compound of claim 1, wherein R⁷ and/or R⁸ is halo.

17. The compound of claim 1, wherein R⁷ and/or R⁸ is NH₂.

18. The compound of claim 1, wherein the compound is of Formula I-A, I-B, I-C, I-D, I-E, II-A, II-B, or II-C: 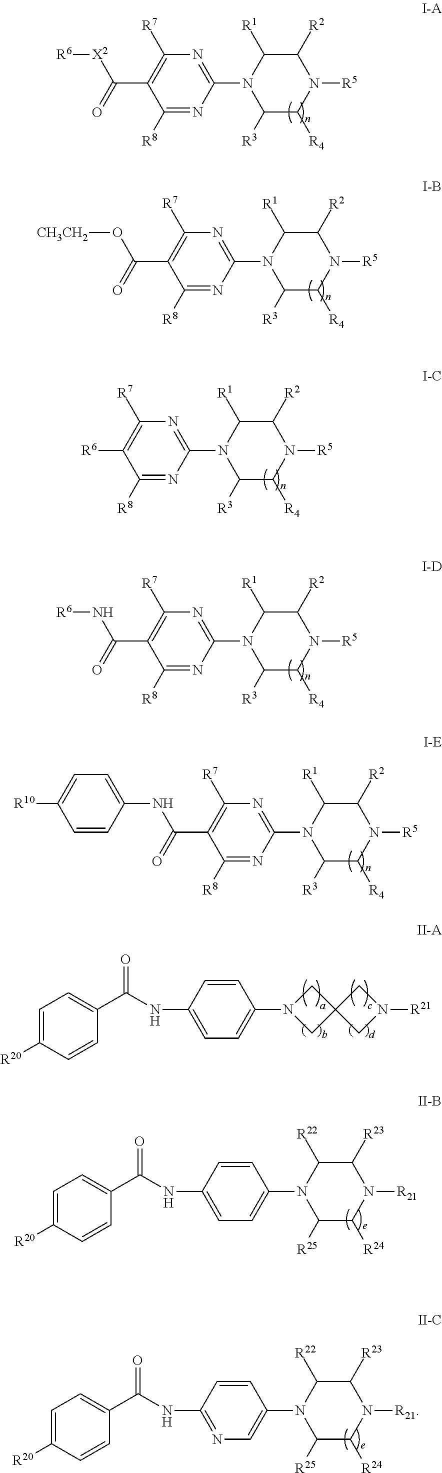
wherein, R¹⁰ is C₁₋₆alkoxy, halo, C₁₋₆trihaloalkyl, or C₁₋₆trihaloalkoxy.

19. The compound of claim 1, that is:
4-amino-N-(4-fluorophenyl)-2-(4-(pyridin-2-yl)piperazin-1-yl)pyrimidine-5-carboxamide (34);
2-hydroxyethyl 4-amino-2-(4-(pyridin-2-yl)piperazin-1-yl)pyrimidine-5-carboxylate (37);
4-amino-2-(4-(pyridin-2-yl)piperazin-1-yl)-N-(4-(trifluoromethyl)phenyl)pyrimidine-5-carboxamide (35);
4-amino-2-(4-(pyridin-2-yl)piperazin-1-yl)-N-(4-(trifluoromethoxy)phenyl)pyrimidine-5-carboxamide (36);
2-(4-(pyridin-2-yl)piperazin-1-yl)pyrimidin-4-amine (40);
4-amino-2-(4-(pyridin-2-yl)piperazin-1-yl)pyrimidine-5-carbonitrile (41);
(4-amino-2-(4-(pyridin-2-yl)piperazin-1-yl)pyrimidin-5-yl)methanol (38);
5-methyl-2-(4-(pyridin-2-yl)piperazin-1-yl)pyrimidin-4-amine (39);
ethyl 4-amino-2-(3-(benzo[d]thiazol-2-yl)-3,8-diazabicyclo[3.2.1]octan-8-yl)pyrimidine-5-carboxylate (12);
ethyl 4-amino-2-(4-(benzo[d]oxazol-2-yl)piperazin-1-yl)pyrimidine-5-carboxylate (10);
ethyl 4-amino-2-(4-(benzo[d]thiazol-2-yl)piperazin-1-yl)pyrimidine-5-carboxylate (11);
4-amino-2-(4-(benzo[d]oxazol-2-yl)piperazin-1-yl)-N-(4-methoxyphenyl)pyrimidine-5-carboxamide (26);
4-amino-2-(4-(benzo[d]thiazol-2-yl)piperazin-1-yl)-N-(4-methoxyphenyl)pyrimidine-5-carboxamide (27);
ethyl 4-amino-2-(4-(4-methoxyphenyl)piperazin-1-yl)pyrimidine-5-carboxylate (17);
ethyl 4-amino-2-(4-(quinoxalin-2-yl)piperazin-1-yl)pyrimidine-5-carboxylate (16);
4-amino-N-(4-methoxyphenyl)-2-(4-(4-methoxyphenyl)piperazin-1-yl)pyrimidine-5-carboxamide (33);
4-amino-N-(4-methoxyphenyl)-2-(4-(quinoxalin-2-yl)piperazin-1-yl)pyrimidine-5-carboxamide (32);
N-(4-methoxyphenyl)-2-(4-(pyridin-2-yl)piperazin-1-yl)pyrimidine-5-carboxamide (42);
ethyl 4-amino-2-(4-(pyridin-2-yl)-1,4-diazepan-1-yl)pyrimidine-5-carboxylate (7);
4-amino-N-(4-methoxyphenyl)-2-(4-(pyridin-2-yl)-1,4-diazepan-1-yl)pyrimidine-5-carboxamide (23);
ethyl 4-amino-2-(4-(pyridin-2-yl)piperazin-1-yl)pyrimidine-5-carboxylate (9);
ethyl 4-amino-2-(8-(pyridin-2-yl)-3,8-diazabicyclo[3.2.1]octan-3-yl)pyrimidine-5-carboxylate (4);
ethyl 4-amino-2-(8-(isoquinolin-3-yl)-3,8-diazabicyclo[3.2.1]octan-3-yl)pyrimidine-5-carboxylate (5);
ethyl 4-amino-2-(8-(quinolin-3-yl)-3,8-diazabicyclo[3.2.1]octan-3-yl)pyrimidine-5-carboxylate (6);
4-amino-N-(4-methoxyphenyl)-2-(4-(pyridin-2-yl)piperazin-1-yl)pyrimidine-5-carboxamide (28);
ethyl 4-amino-2-(3-(pyridin-2-yl)-3,8-diazabicyclo[3.2.1]octan-8-yl)pyrimidine-5-carboxylate (1);
4-amino-2-(8-(isoquinolin-3-yl)-3,8-diazabicyclo[3.2.1]octan-3-yl)-N-(4-methoxyphenyl)pyrimidine-5-carboxamide (21);
4-amino-N-(4-methoxyphenyl)-2-(8-(quinolin-3-yl)-3,8-diazabicyclo[3.2.1]octan-3-yl)pyrimidine-5-carboxamide (22);
4-amino-N-(4-methoxyphenyl)-2-(3-(pyridin-2-yl)-3,8-diazabicyclo[3.2.1]octan-8-yl)pyrimidine-5-carboxamide (18);
ethyl 4-amino-2-(3-(isoquinolin-3-yl)-3,8-diazabicyclo[3.2.1]octan-8-yl)pyrimidine-5-carboxylate (2);
ethyl 4-amino-2-(3-(quinolin-3-yl)-3,8-diazabicyclo[3.2.1]octan-8-yl)pyrimidine-5-carboxylate (3);
ethyl 4-amino-2-(4-(quinolin-3-yl)-1,4-diazepan-1-yl)pyrimidine-5-carboxylate (8);
4-amino-2-(3-(isoquinolin-3-yl)-3,8-diazabicyclo[3.2.1]octan-8-yl)-N-(4-methoxyphenyl)pyrimidine-5-carboxamide (19);
4-amino-N-(4-methoxyphenyl)-2-(3-(quinolin-3-yl)-3,8-diazabicyclo[3.2.1]octan-8-yl)pyrimidine-5-carboxamide (20);
4-amino-N-(4-methoxyphenyl)-2-(4-(quinolin-3-yl)-1,4-diazepan-1-yl)pyrimidine-5-carboxamide (25);
4-amino-2-(4-(isoquinolin-3-yl)-1,4-diazepan-1-yl)-N-(4-methoxyphenyl)pyrimidine-5-carboxamide (24);
ethyl 4-amino-2-(4-(quinolin-3-yl)piperazin-1-yl)pyrimidine-5-carboxylate (14);
ethyl 4-amino-2-(4-(quinolin-2-yl)piperazin-1-yl)pyrimidine-5-carboxylate (15);
4-amino-N-(4-methoxyphenyl)-2-(4-(quinolin-3-yl)piperazin-1-yl)pyrimidine-5-carboxamide (30);
4-amino-N-(4-methoxyphenyl)-2-(4-(quinolin-2-yl)piperazin-1-yl)pyrimidine-5-carboxamide (31);
ethyl 4-amino-2-(4-(isoquinolin-3-yl)piperazin-1-yl)pyrimidine-5-carboxylate (13);
4-amino-2-(4-(isoquinolin-3-yl)piperazin-1-yl)-N-(4-methoxyphenyl)pyrimidine-5-carboxamide (29);
ethyl 4-amino-2-(4-(pyridin-4-yl)piperazin-1-yl)pyrimidine-5-carboxylate (117);
4-amino-N-(4-methoxyphenyl)-2-(4-(pyridin-3-yl)piperazin-1-yl)pyrimidine-5-carboxamide (118);
ethyl 4-amino-2-(4-(pyrimidin-2-yl)piperazin-1-yl)pyrimidine-5-carboxylate (119);
4-amino-N-(4-methoxyphenyl)-2-(4-(pyrimidin-2-yl)piperazin-1-yl)pyrimidine-5-carboxamide (120);
ethyl 4-amino-2-(4-(pyrazin-2-yl)piperazin-1-yl)pyrimidine-5-carboxylate (121);
4-amino-N-(4-methoxyphenyl)-2-(4-(pyrazin-2-yl)piperazin-1-yl)pyrimidine-5-carboxamide (122);
ethyl 4-amino-2-(4-(thiazol-2-yl)piperazin-1-yl)pyrimidine-5-carboxylate (123);
4-amino-N-(4-methoxyphenyl)-2-(4-(thiazol-2-yl)piperazin-1-yl)pyrimidine-5-carboxamide (124);
ethyl 4-amino-2-(3-(benzo[d]thiazol-2-yl)-3,8-diazabicyclo[3.2.1]octan-8-yl)pyrimidine-5-carboxylate (125);
4-amino-N-(4-methoxyphenyl)-2-(8-(quinolin-3-yl)-3,8-diazabicyclo[3.2.1]octan-3-yl)pyrimidine-5-carboxamide (126);
4-amino-N-(2-fluoro-4-methoxyphenyl)-2-(4-(pyridin-2-yl)piperazin-1-yl)pyrimidine-5-carboxamide (127);
4-amino-N-(3-fluoro-4-methoxyphenyl)-2-(4-(pyridin-2-yl)piperazin-1-yl)pyrimidine-5-carboxamide (128);
2-hydroxyethyl 4-amino-2-(4-(pyridin-2-yl)piperazin-1-yl)pyrimidine-5-carboxylate (129);
N-(4-methoxyphenyl)-2-(4-(pyridin-2-yl)piperazin-1-yl)pyrimidine-5-carboxamide (130);
4-amino-N-(4-hydroxyphenyl)-2-(4-(pyridin-2-yl)piperazin-1-yl)pyrimidine-5-carboxamide (131);
4-amino-N-(4-(2-fluoroethoxy)phenyl)-2-(4-(pyridin-2-yl)piperazin-1-yl)pyrimidine-5-carboxamide (132);
4-amino-2-(4-(benzo[d]oxazol-2-yl)piperazin-1-yl)-N-(4-(2-fluoroethoxy)phenyl)pyrimidine-5-carboxamide (133);
4-methoxy-N-(4-(6-(pyridin-2-yl)-2,6-diazaspiro[3.3]heptan-2-yl)phenyl) benzamide (117);
4-methoxy-N-(4-(7-(pyridin-2-yl)-2,7-diazaspiro[3.5]nonan-2-yl)phenyl) benzamide (118);
4-methoxy-N-(4-(7-(3-nitrophenyl)-2,7-diazaspiro[3.5]nonan-2-yl)phenyl) benzamide (119);
4-methoxy-N-(4-(7-(pyridin-2-yl)-2,7-diazaspiro[4.4]nonan-2-yl)phenyl) benzamide (120);
4-methoxy-N-(4-(5-(pyridin-2-yl)hexahydropyrrolo[3,4-b]pyrrol-1(2H)-yl)phenyl) benzamide (100);
4-methoxy-N-(4-(2-(pyridin-2-yl)-2,8-diazaspiro[4.5]decan-8-yl)phenyl) benzamide (101);
4-methoxy-N-(4-(5-(pyridin-2-yl)-2,5-diazabicyclo[2.2.1]heptan-2-yl)phenyl) benzamide (102);
4-methoxy-N-(4-(3-(pyridin-2-yl)-3,8-diazabicyclo[3.2.1]octan-8-yl)phenyl) benzamide (103);
4-methoxy-N-(4-(3-(quinolin-3-yl)-3,8-diazabicyclo[3.2.1]octan-8-yl)phenyl) benzamide (104);
4-methoxy-N-(4-(3-(quinoxalin-2-yl)-3,8-diazabicyclo[3.2.1]octan-8-yl)phenyl) benzamide (105);
4-methoxy-N-(4-(3-(pyridin-2-yl)-3,9-diazabicyclo[3.3.1]nonan-9-yl)phenyl) benzamide (106);
4-methoxy-N-(4-(4-(pyridin-2-yl)-1,4-diazepan-1-yl)phenyl)benzamide (107);
N-(4-(4-(5-chloropyridin-2-yl)piperazin-1-yl)phenyl)-4-methoxybenzamide (108);
4-methoxy-N-(5-(4-(pyridin-2-yl)piperazin-1-yl)pyridin-2-yl)benzamide (109);
4-methoxy-N-(5-(3-(pyridin-2-yl)-3,8-diazabicyclo[3.2.1]octan-8-yl)pyridin-2-yl) benzamide (110);
4-methoxy-N-(5-(4-(pyridin-2-yl)-1,4-diazepan-1-yl)pyridin-2-yl)benzamide (111);
4-methoxy-N-(6-(3-(pyridin-2-yl)-3,8-diazabicyclo[3.2.1]octan-8-yl)pyridin-3-yl) benzamide (112);
4-methoxy-N-(4-(4-methylpiperazin-1-yl)phenyl)benzamide (113);
N-(4-(1-azaspiro[3.5]nonan-1-yl)phenyl)-4-methoxybenzamide (114);
4-methoxy-N-(4-(3-methoxyazetidin-1-yl)phenyl)benzamide (115);
4-(2-fluoroethoxy)-N-(4-(3-(pyridin-2-yl)-3,8-diazabicyclo[3.2.1]octan-8-yl)phenyl) benzamide (116);
or a pharmaceutically acceptable salt or isotopic variant thereof.

20-41. (canceled)

42. The compound of claim 1, wherein the compound of Formula II is labeled with one or more of a non-radioactive isotope.

43. A pharmaceutical composition comprising one or more compounds of claim 1.

44. A method of modulating alpha-synuclein in a patient in need thereof, comprising administering an effective amount of the compound of claim 1 to the patient.

45-46. (canceled)

47. A method of treating a neurodegenerative disorder in a patient in need thereof, comprising:
administering a pharmaceutical agent that clears aggregated alpha-synuclein from the brain of the patient;
administering an effective amount of a compound of claim 1 to the patient; and
performing positron emission tomography imaging on the patient.

48-51. (canceled)

52. The compound of claim 1, wherein R⁶ is optionally substituted phenyl.
